(19) United States
(12) Patent Application Publication
(10) Pub. No.: US 2016/0231324 A1
(43) Pub. Date: Aug. 11, 2016

(54) ENCAPSULATED SENSORS AND SENSING SYSTEMS FOR BIOASSAYS AND DIAGNOSTICS AND METHODS FOR MAKING AND USING THEM

(71) Applicant: THE REGENTS OF THE UNIVERSITY OF CALIFORNIA, Oakland, CA (US)

(72) Inventors: Weian ZHAO, Irvine, CA (US); Dong-ku KANG, Irvine, CA (US); Kaixiang ZHANG, Irvine, CA (US); Md Monsur ALI, Irvine, CA (US); Mark A. ECKERT, Irvine, CA (US); Feng LI, Irvine, CA (US); Enrico GRATTON, Irvine, CA (US); MIchelle A. DIGMAN, Irvine, CA (US); Louai LABANIEH, Irvine, CA (US); Mengrou LU, Irvine, CA (US)

(21) Appl. No.: 15/024,735

(22) PCT Filed: Sep. 24, 2014
(86) PCT No.: PCT/US14/57282

§ 371 Date: Mar. 24, 2016

Related U.S. Application Data
(60) Provisional application No. 61/881,827, filed on Sep. 24, 2013.
(60) Provisional application No. 61/987,078, filed on May 1, 2014.

(51) Int. Cl. G01N 33/574; G01N 33/569

(52) U.S. Cl. CPC G01N 33/57496; G01N 33/56911; G01N 33/56916

(57) ABSTRACT

In alternative embodiments, the invention provides high throughput, multiplexed systems or methods for detecting a biological, a physiological or a pathological maker, or a single molecule or a single cell using a droplet microfluidics system integrated with use of a sensor or a sensing system, an aptamer, or a DNAzyme. In alternative embodiments, the sensor or sensing system comprises a nucleic acid based, an antibody based, an enzyme based or a chemical based sensor or sensing system. In alternative embodiments, the invention provides methods for detecting a biological, a physiological or a pathological marker, or a single molecule or a single cell using a droplet system integrated with rapid and sensitive fluorescence detection systems including, for example, a 3D Particle Detector. In alternative embodiments, the invention provides systems comprising Integrated Comprehensive Droplet Digitial Detection (IC 3D).

### TECHNICAL FIELD

This invention generally relates to bioanalysis, and detection and screening methodologies. In particular, in alternative embodiments, the invention provides high throughput, multiplexed systems or methods for detecting a biological, a physiological or a pathological marker, or a single molecule or a single cell using a droplet microfluidics system, or an emulsifier, integrated with use of a sensor or a sensing system, an aptamer, or a DNAzyme. In alternative embodiments, the sensor or sensing system comprises a nucleic acid based, an antibody based, an enzyme based or a chemical based sensor or sensing system. In alternative embodiments, the invention provides methods for detecting a biological, a physiological or a pathological marker, or a single molecule or a single cell using a droplet or emulsion system integrated with rapid and sensitive fluorescence detection systems including, in particular, a 3D Particle Detector. In alternative embodiments, the invention presents methods for high throughput screening of small molecules and biomolecules, including aptamers, such as oligonucleic acid and peptide aptamers, and related, e.g., aptamer-based, sensors and therapeutics.

### BACKGROUND

Recent advances in genomics, proteomics, cellomics and metabolomics have provided us with large libraries of biological and chemical compounds that modulate various biological processes. Such developments have necessitated the need for high throughput analysis/screening where millions of biochemical, genetic or pharmacological assays are performed and analyzed in a parallel fashion to find active compounds against biological targets. In addition, the analysis, detection, identification and quantification of these markers provide powerful new means to study biology and pathology and to develop new diagnostics and therapeutics.

Many biological and disease markers, such as e.g., molecules and cells such as cancer cells, exist at low concentrations in biological samples, yet play important roles in biological and pathological processes. The ability to rapidly and selectively detect low abundance is critically important to elucidate new biology, to monitor, detect a disease or disorder, and to monitor therapeutic responses and to develop new therapeutics.

Early identification, screening and monitoring of cancer, Alzheimer's Disease (AD) and other diseases and conditions, e.g., before a person has any symptoms, has proven to be a powerful and often necessary step to effectively prevent, treat and eradicate the disease. Traditional imaging tools (e.g., computed tomography (CT) scans and magnetic resonance imaging (MRI)) and biopsy analysis are unfortunately too complicated, expensive and/or invasive for routine disease screening; most importantly, they typically do not possess the sensitivity and specificity to identify the diseases at the early stage. Therefore, recent effort has been focused on developing assays targeting specific molecular biomarkers (e.g., nucleic acids and proteins) and cellular markers (e.g., cancer cells) existed in biological samples (e.g., blood, urine, saliva, tear, and cerebrospinal fluid (CSF)) that distinguish disease from normal samples.

Unfortunately, discovering disease biomarkers and translating them into clinical assays has proven to be an enormous challenge. First, despite the advances in genomic and proteomic technologies (e.g., sequencing, mass spectrometry (MS), and bioinformatics) which are sophisticated and costly, very few reliable disease biomarkers have been discovered. These technologies are limited by their intrinsic, high false discovery rate and the fact that modest differences between normal and diseased samples and large heterogeneity of biomarkers in the diseased samples exist. It has widely been accepted that a single biomarker typically lacks the sensitivity and specificity that is necessary for useful diagnosis. Additionally, even once biomarkers are identified, the implementation and clinical assay development in the next phase is also time-consuming, expensive and sometimes infeasible. For instance, if one wants to develop an ELISA assay to detect prostate-specific antigen (PSA) as a biomarker for prostate cancer, the antibodies for PSA have to already exist with sufficient specificity and selectivity. This is particularly problematic when multiple biomarker assays are required.

Another important area that requires sensitive, rapid and high throughput biomarker identification and detection is infections by pathogens (e.g., bacteria such as tuberculosis (TB), viruses (e.g., HIV), and parasites such as malaria). For instance, bacterial infection is a major health problem and a major cause of sepsis, which annually affects over 18 million people worldwide and 700,000 in the U.S., with a mortality rate of 30-40%. Sepsis and other aggressive bacterial infections are managed within intensive care units with associated high costs, which impose significant healthcare, economic and social burdens. For instance, each septic patient in the US incurs costs of approximately US $25,000 during hospitalization, corresponding to $17 billion annually. In particular, antimicrobial resistance is a growing health problem in the United States and worldwide. According to the Centers for Disease Control and Prevention (CDC), more than two million people are infected annually with antibiotic-resistant infections, with greater than 23,000 deaths¹. Aggressive bacterial infections associated with antimicrobial resistance are often managed within intensive care units (ICUs) with high associated costs, which impose significant healthcare, economic and social burdens. The Alliance for the Prudent Use of Antibiotics (APUA) estimates the antibiotic-resistant infections cost the US healthcare system over $20 billion each year.

The high mortality of blood infections is associated with the ineffectiveness and time-consuming process of bacteria diagnosis and treatment. It is widely recognized that effective detection and routine monitoring of infectious bacteria in patients to diagnose diseases at an early-stage have a profound effect on survival rates. Unfortunately, blood culture, the gold standard for identification of bacteria in blood, takes days to obtain results. New molecular diagnosis methods, such as polymerase chain reaction (PCR), can reduce the assay time to hours but are often not sensitive enough to detect bacteria that occur at low concentrations in blood (1-100 colony-forming unit (CFU)/mL). Importantly, PCR-based methods require sample processing, such as lysis and isolation of nucleic acids, for the amplification reaction. Moreover, all these techniques are sophisticated and expensive, and therefore not suited for routine monitoring of bacteria in patients. Therefore, simple methods are urgently needed for rapid and sensitive identification of bacteria in blood, which will significantly reduce the mortality rate and the cost of medical care associated with blood infections.

Microfluidic systems have recently emerged as a promising platform for performing a diverse range of experiments for biological and chemical applications. Microfluidic-based methods have several advantages compared to conventional high throughput screening methods. These include negligible evaporation of reagents, minimal consumption of expensive biological reagents, low fabrication costs, reduced analysis time and the ability to integrate various functional components on a single chip.

In particular, the developments of droplet based microfluidic systems present a promising opportunity for high-throughput biological analysis. In these systems, microdroplets containing nano- to picolitre volumes can be generated at kilohertz frequencies and each droplet serves as a ‘test tube’ for reactions. Because of the small volume of each droplet, reactions between bio-molecules such as protein-protein interaction or DNA hybridization and cell-drug or cell-cell interactions can be performed using 10⁹ times smaller amounts than conventional biological methods such as 96 microwell plate based Enzyme-linked immunosorbent assay (ELISA). In addition, droplet confinement of targets e.g., cells and its immediate environment into a small volume allows us to analyze secreted markers and use them as “markers” for single cell detection and sorting. By contrast, existing techniques, e.g., ELISA, typically measure secreted proteins in bulk and therefore miss key dynamic information at a single cell level. Fluorescence activated cell sorting (FACS) typically rely on cell surface and intracellular markers, rather than secreted markers, for cell sorting. Furthermore, droplet based microfluidic systems have additional advantages compared to continuous microfluidic systems such as reducing the reagent interaction with channel walls and inhibiting dispersion of samples by compartmentalization. In addition, it allows independent control of each droplet including droplet generation, coalescence, sorting, incubation and analysis in a short period of time.

### SUMMARY

In alternative embodiments, the invention provides high throughput, multiplexed systems or devices, or methods, for detecting, identifying and/or quantifying a target; a target molecule; a virus; a biological, a physiological or a pathological marker; a single molecule; or a single cell or cell-derived particle, e.g., a single pathogen, parasite, bacterial cell, virus or fungus, using a droplet or emulsion-based microfluidics system, a 3D particle detector and/or a 3D particle counting system, integrated with use of an assay, a sensor or a sensing system comprising use of: a small molecule, a biomolecule, an aptamer, a DNAzyme, a nucleic acid, a protein, a peptide, an enzyme, an antibody, or a chemical or small molecule, comprising:

(a) providing an assay, a sensor, a detecting or a sensing system capable of specifically binding to or detecting directly or indirectly a target, a target molecule, a nucleic acid, a protein, a peptide, a virus (e.g., a lentivirus such as HIV, or Ebola virus disease (EVD)), a cell-derived particle or a cell, wherein optionally the cell is a bacterial cell (optionally a slowly-growing organism such as Mycobacterium tuberculosis), a parasite cell or a fungal cell, or optionally the cell is a mammalian cell or a human cell;

wherein optionally the assay, sensor, detecting or sensing system comprises or comprises use of: an aptamer, a DNAzyme (also called a deoxyribozyme, a DNA enzyme or a catalytic DNA), a nucleic acid, a protein, a peptide, an enzyme, an antibody, a chemical or small molecule, a single nucleic acid molecule amplification optionally comprising an EXPonential Amplification Reaction (EXPAR), a Rolling Circle Amplification (RCA), an aptamer Inhibitor-DNA-Enzyme (IDE), or an aptamer-IDE system,

and optionally the target comprises an amplified target, which optionally is a nucleic acid target amplified using Rolling Circle Amplification (RCA) or EXPAR,

wherein the specific binding to, or the direct or indirect detecting of, the target molecule, virus, cell-derived particle or cell, by the assay, sensor, detecting or sensing system results in, or generates, a detectable signal, which optionally comprises a fluorophore signal or a fluorescence,

wherein optionally the nucleic acid, aptamer, aptamer-IDE system, or DNAzyme comprises a RNA-cleaving DNA motif that can cleave a DNA-RNA chimeric substrate at a single ribonucleotide junction, and the ribonucleotide cleavage site is flanked by a fluorophore and a quencher, and optionally binding of the nucleic acid, aptamer, or DNAzyme to its target molecule, virus, cell-derived particle or cell causes cleavage of a ribonucleotide cleavage site to release the quencher from the fluorophore or a fluorescence activator, wherein the fluorescence activator optionally comprises an enzyme capable of when in active form generating a detectable signal such as a fluorophore signal,

and optionally the sensor or sensing system, aptamer, a DNAzyme, an aptamer inhibitor-DNA-enzyme (IDE) molecular complex (also called an aptamer-IDE system), which optionally comprises a structure as set forth in FIG. 47, wherein the enzyme of the IDE molecular complex when active (e.g., not under the influence of an inhibitor) can generate a detectable signal such as a fluorescent signal when uninhibited, and the enzyme of the IDE molecular complex is inhibited by the inhibitor of the IDE molecular complex with the IDE molecular complex is not bound to a target, and the inhibitor of the IDE molecular complex is released, removed or deactivated from the enzyme when the aptamer of the IDE molecular complex binds its target, thus triggering activation of the enzyme and triggering the generation of the detectable signal, e.g., the fluorescent signal,

and optionally the assay, sensor, detecting or sensing system comprises a nucleic acid based, an antibody based, a protein based, a peptide based, an enzyme based or a chemical or small molecule-based assay, sensor, detecting or sensing system, or any combination thereof,

wherein optionally the specific binding of the assay, sensor, detecting or sensing system, to the target triggers an amplification-based or non-amplification-based fluorescence signal,

and optionally the target molecule (optionally a purified or complex target) can be screened, selected and/or isolated from a nucleic acid, peptide or chemical library,

and optionally the target molecule comprises a nucleic acid or a polypeptide, optionally the polypeptide is a diagnostic for a disease or condition, or is a cell surface marker, or is an enzyme, wherein optionally the enzyme is a marker for the detection of a particular disease or is a marker, optionally the enzyme is a beta-lactamase, such as a carbapenemase, optionally for the detection of extended spectrum beta-lactamase (ESBL)-producing Enterobacteriaceae and carbapenem-resistant Enterobacteriaceae (CRE), TB and other antimicrobial resistant pathogens,

and optionally the target molecule, virus, cell-derived particle or cell or bacteria, parasite or fungus, comprises one or a plurality of biological, physiological or pathological markers, or comprises a single or a plurality of molecules or a single cell or a plurality of cells, or a single or a plurality of virus or a cell-derived particles or molecules;

(b) optionally providing a plurality of droplets or microdroplets, or emulsions,

wherein optionally the droplets or microdroplets, or emulsions, are generated by a droplet microfluidics system or a microdroplet-manipulating assay or device, or an emulsifier, or an equivalent device or system,

and optionally droplet size can range from between about 5 to 50 μm in diameter, between about 1 μm to 300 μm, or between about 10 μm to 100 μm,

and optionally providing labels or stains, wherein optionally the target or the amplified target are stained or labeled, optionally with a dye, a nanoparticle, a bead, or an equivalent or combination thereof,

and optionally providing a plurality of particles or nanoparticles, wherein the target consists of, comprises or is contained in the particles or nanoparticles;

(c) providing a sample, wherein optionally the sample comprises or is derived from a biological or an environmental sample,

and optionally the sample comprises the target, or is suspected of comprising the target to be detected,

and optionally the target is or comprises a target molecule, a nucleic acid, a protein, a peptide, a virus, a cell-derived particle or a cell, wherein optionally the cell is a bacterial cell, a parasite cell or a fungal cell, or optionally the cell is a mammalian cell or a human cell;

(d) optionally encapsulating or microencapsulating the sample (comprising or consisting of the target), optionally together with the assay, sensor, detecting or sensing system,

and optionally associating, encasing, or binding the target or the sample with or within the plurality of particles or nanoparticles,

wherein optionally the encapsulating or microencapsulating comprises encapsulating or microencapsulating into a plurality of droplets or microdroplets, or emulsions,

and optionally the target-detecting or sensing system comprises an aptamer-IDE system, and optionally when the aptamer-IDE system comprises use of an enzyme, or a combination of enzymes, that can generate a detectable signal, such as a fluorescent signal, by interacting or processing the detectable signal, the encapsulating or microencapsulating further comprises encapsulating or microencapsulating a substrate or a detectable signal activated by the enzyme,

and optionally processing or making the encapsulated or microencapsulated sample or target, or processing or making the droplets or microdroplets, or emulsions, comprising the encapsulated or microencapsulated sample, comprises use of a droplet microfluidics system or microdroplet-manipulating device, or a high-throughput droplet generator, optionally a 256 channel cartridge system, or an emulsifier,

and optionally labeling or staining the target or the amplified target, optionally with a dye, a nanoparticle, a bead, or an equivalent or combination thereof, and

(e) detecting the presence of a detectable signal, which optionally comprises a fluorophore signal or a fluorescence, or a dye, a nanoparticle, a bead, or an equivalent or combination thereof,

wherein optionally the detecting, identifying and/or quantifying of the presence of a detectable signal is in each encapsulated or microencapsulated sample, or in each droplet or microdroplet, or emulsion, or is in each particle or nanoparticle,

and optionally detecting the presence of a detectable signal detects, identifies and/or quantifies the target molecule, virus, cell-derived particle or cell, wherein optionally the cell is a mammalian cell, a human cell, a bacterial cell, a parasite cell, a fungal cell,

wherein the detection of a fluorophore signal or fluorescence, which optionally is in an encapsulated or microencapsulated sample, or a droplet or microdroplet, or an emulsion, or is in each particle or nanoparticle, indicates the presence of the target molecule, virus, cell-derived particle, cell, parasite, fungus or mammalian or human cell in the sample,

and optionally the detecting and/or quantifying the target molecule, a virus or a cell-derived particle or a cell comprises use of a 3D particle detector or a 3D particle counting system.

In alternative embodiments, the target detected is encapsulated (or microencapsulated) within a droplet or microdroplet or an emulsion, or is associated with or within a particle or a nanoparticle, or alternatively, the target (which can be, for example, in addition to a droplet or microdroplet, a bead, a nanoparticle, an amplified nucleic acid, an inhibitor-DNA-enzyme (IDE) molecular complex, and equivalents) is/are directly detected and/or counted by the 3D particle detector, 3D particle counting system, or equivalent system; e.g., as illustrated in FIG. 8.

In alternative embodiments, the cell is a mammalian cell, a human cell, a circulating tumor cell, a circulating melanoma cell, or a bacterial cell.

In alternative embodiments, the droplet microfluidics system, or emulsifier, can generate: (a) picoliter droplets or droplets of between about 1 μm to 300 μm, or between about 10 μm to 100 μm, in diameter; and/or (b) monodisperse, picoliter-sized liquid droplets in an immiscible carrier oil fluid.

In alternative embodiments, the biological sample comprises a biopsy, a blood, serum, saliva, tear, urine or a CSF sample from a patient, or a sample obtained from a food, water, soil, or an air source.

In alternative embodiments, the target molecule detected is or comprises a nucleic acid, a nucleic acid point mutation, or a single-nucleotide polymorphism (SNP), or a microRNA (miRNA) or a small inhibitory RNA (siRNA); or, the target molecule is a protein, a lipid, a carbohydrate, a polysaccharide, a small molecule or a metal complex.

In alternative embodiments, the target molecule is or comprises a polypeptide or a nucleic acid, a polypeptide or a nucleic acid point mutation, or a single-nucleotide polymorphism (SNP), a cell marker (a marker specific or identifying for a particular cell type, genotype or phenotype); or a nucleic acid disease (e.g., diabetes, Alzheimer's disease, and the like) or cancer marker, optionally a breast cancer biomarker,

and optionally detection of the target molecule is diagnostic for the disease (e.g., diabetes, Alzheimer's disease, and the like) or cancer (e.g., prostate, melanoma, breast cancer, optionally the target is prostate-specific antigen (PSA)), or is used for routine disease or cancer screening, early stage disease or cancer diagnosis and/or prognosis, for monitoring disease or cancer progression and/or recurrence, and/or for monitoring drug effectiveness and safety.

In alternative embodiments, the fluorophore comprises a fluorescein-dT and the quencher is a DABCYL-dT™ (Dabcyl-dT); and/or a fluorescence resonance energy transfer (FRET) dye pair; and/or a target-binding dye.

In alternative embodiments, the fluorescence is detected by an APD (photon avalanche diode), a PMT (photomultiplier tubes), a EMCCD (Electron Multiplying Charge Coupled Device), or a MCP (Microchannel plate) or other equivalent detector, optionally in a high throughput manner.

In alternative embodiments, the aptamer is an oligonucleotide, a nucleic acid or a peptide aptamer; or, the aptamer: specifically modulates stem cell differentiation into a particular lineage, or is directly coupled to a downstream signaling pathway.

In alternative embodiments, the aptamer binds to a target as agonist or antagonist or turns on a fluorescence signal as a sensor.

In alternative embodiments, the sensor comprises a DNA strand displacement strategy, or equivalents, as described e.g., in Li et al. (2013) J. Am. Chem. Soc. 2013, 135, 2443-2446; or a proximity ligation assay, or a binding induced DNA assembly assay, as described e.g., in Li et al. (2012) Angew. Chem., Int. Ed. 51, 9317; or Zhang (2012) Anal. Chem. 84:877.

In alternative embodiments, the sensor comprises a fluorogenic substrate or probe, or equivalents that binds to a target to produce fluorescence.

In alternative embodiments, the high throughput, multiplexed system or device, or method, of the invention further comprise detecting and/or quantifying the target, e.g., one or a plurality of biological, physiological or pathological markers, or a single molecule (as the target), or a single cell integration, comprising use of a 3D particle detector, a 3D particle counting system, or equivalent systems. In alternative embodiments, the target detected is encapsulated (or microencapsulated) within a droplet or microdroplet or an emulsion, or is associated with or within a particle or a nanoparticle, or alternatively, the target is directly detected and/or counted by the 3D particle detector, 3D particle counting system, or equivalent system. In alternative embodiments, the high throughput, multiplexed system or device, or method, of the invention comprise use of a DNA-bead or a DNA-bead droplet library or FACS based screening for molecules that bind to a target of interest, for example, a disease or a cancer cell, or a disease or a cell marker, e.g., a nucleic acid or a polypeptide, e.g., a membrane, marker.

In alternative embodiments, the high throughput, multiplexed system is engineered to comprise one or any of: desirable portability (for example, packaged as backpacks), automating fluid handing (i.e., droplet generation and auto sampling), and integrating electronics including a diode laser (light source), APD (detector), Operating (vinci, ISS Inc.) and/or data analyzing software (SimFCS), display, with a 3D particle counting system, e.g., as illustrated in FIGS. 32, 33 and 40, illustrating an exemplary portable system design of the invention comprising integrated micro-encapsulator and 3D particle counting system.

In alternative embodiments, the high throughput, multiplexed system or device, or method, of the invention further comprise disposable microfluidic “cartridges,” permitting multiplex and rapid detection of multiple types of targets simultaneously, and optionally the high throughput, multiplexed system or device is fully automated, or is fabricated as an all-in-one system or with modular components, or is linked to an electronic device, e.g., a portable device, e.g., a smart phone and/or a Bluetooth, for point-of-care applications, as illustrated in FIGS. 32, 33 and 40.

In alternative embodiments of the high throughput, multiplexed system or device, or method, of the invention, the assay, sensor or sensor system comprises: a nucleic acid based assay; an antibody based assay; an enzyme based assay; a chemical based assay; a nucleic acid based assay; a hybridization; a molecular beacon; an aptamer; a DNAzyme; a real-time fluorescent sensor; an antibody-based assay; an ELISA; a sandwich based assay; an immunostaining assay; an antibody capture assay; a secondary antibody amplification assay; a proximity ligation based assay; an enzyme based assay comprising use of a PCR, RT-PCR, RCA, loop-mediated isothermal amplification (LAMP), nicking, strand displacement and/or an exponential isothermal amplification; or any combination thereof,

wherein optionally the high throughput, multiplexed system or device, or method detects low concentration targets without using droplets,

and optionally nucleic acid targets are detected using signal amplification processes, optionally Rolling Circle Amplification (RCA),which are then stained by dye probes or nanoparticles and measured, optionally by a 3D particle counter.

In alternative embodiments of the high throughput, multiplexed system or device, or method, of the invention, the encapsulated or microencapsulated emulsions or droplets are made by using an emulsifier or by droplet based microfluidics; or the emulsions or droplets comprise water-in-oil formulations, or the droplets comprise water-in-oil-in-water (W/O/W) double emulsion formulations, or the emulsions or droplets comprise liquid droplets, optionally comprising an agarose or a PEG, or optionally the droplets can be gelled or solidified to form droplet particles;

and optionally droplets comprise sizes ranging from between about 10 nm to 100 microns, optionally droplets are monodispersed or polydispersed, and optionally droplets are heated or cooled (e.g., for PCR), merged, split, sorted and/or prepared for long-term storage,

and optionally the emulsions or droplets, optionally fluorescent emulsions or droplets, that contain a target are sorted in a 3D particle counting system, optionally using an optical tweezer, an optical trap, an optical lattice, gradient centrifugation or any combination or an equivalent thereof This enables the sorted target(s) to be further processed and analyzed,

and optionally droplets are analyzed by conventional 1D on-chip or 2D analysis, or by a 3D particle counter.

In alternative embodiments of the high throughput, multiplexed system or device, or method, of the invention, the cell-derived particle comprises an exosome, a microvesicle, an apoptotic body, or any combination thereof; or the target molecule comprises a nucleic acid, a protein, a peptide, a carbohydrate, a lipid, a small molecules, or a metal ion.

In alternative embodiments the invention provides methods of identifying and isolating an enzyme-based target detection system for high through-put detection of specific target, comprising:

(a) providing a library of enzyme-based target detection system molecules designed to bind to and detect one specific target or a plurality of specific targets, the target to which the enzyme-based target detection system designed to detect, and a substrate comprising a detectable moiety,

wherein when the enzyme-based target detection system is not bound to its target, the enzyme is inactive, and when the enzyme-based target detection system binds to its specific target, the enzyme is activated to act on the substrate to generate a detectable signal,

wherein optionally the generated detectable signal comprises a fluorescent signal, and optionally the enzyme-based target detection system is an aptamer inhibitor-DNA-enzyme (IDE) system molecule, optionally as illustrated in FIG. 47 or FIG. 51A,

and optionally the enzyme-based target detection system is a nucleic acid initiator triggered signal amplification cascade, optionally as illustrated in FIG. 50;

(b) encapsulating the sample, an enzyme-based target detection system and substrate in an immiscible carrier oil fluid such that the encapsulation generates a plurality of droplets, wherein droplet each comprises a plurality of sample, an enzyme-based target detection system and substrate,

wherein optionally the encapsulating comprises pumping the sample, an enzyme-based target detection system and substrate through an oil stream, and optionally the plurality of droplets are picoliter sized droplets;

(c) passing the plurality of droplets generated in (b) through a sorter, which directs the droplets having a detectable signal into a separate channel where the sorted droplets are lysed or broken, diluted, and re-encapsulated with additionally added target and substrate at a concentration of about 1 enzyme-based target detection system molecule per drop with in each droplet one or more of substrate and target,

wherein optionally the sorted droplets are lysed or broken optionally using an optical tweezer, an optical trap, an optical lattice, gradient centrifugation or any combination or an equivalent thereof,

wherein optionally the generated detectable signal comprises a fluorescent signal and the sorter is a FACS,

and optionally the generated detectable signal comprises a fluorescent signal and the sorter is a microfluidic device; and

(d) further sorting out droplets having a detectable signal into a separate channel,

thereby identifying and isolating an enzyme-based target detection system or molecule for high through-put detection of the specific target,

wherein optionally the enzyme-based target detection system or molecule comprises a aptamer inhibitor-DNA-enzyme (IDE) system molecule and the isolated IDE molecule is sequenced.

In alternative embodiments, the invention provides drug or aptamer screening and in vitro selection platforms based on one type of molecule/one bead or one type of molecule/one droplet strategy, wherein DNA, RNA, polypeptides and/or peptides are synthesized in a droplet library, comprising:

providing a high throughput, multiplexed system or device, or method, of the invention, and DNA on microbeads for generating a target or a binder to a target,

wherein the DNA on microbeads, or DNA-bread library, is used for screening drug or aptamer that possesses a function, e.g., binding to target molecule or modulate a molecular or cellular function, and optionally wherein the DNA on microbeads is encapsulated in the droplets or microdroplets, optionally picoliter droplets, optionally about 20 μm in diameter,

amplifying the on-bead DNA by PCR to generate a droplet DNA library,

transcribing and/or translating within the droplets the amplified DNA to form RNA and/or polypeptide or peptide libraries,

optionally the identity/sequence of transcribed RNA, and/or the translated polypeptides or peptides, are barcoded in the same droplet using the nucleic acid sequences, for subsequent screening and biomarker discovery,

and optionally the RNA and/or polypeptides or peptides are detected and/or quantified as the target using the high throughput, multiplexed system or device, or method, of the invention.

In alternative embodiments the invention provides an Integrated Comprehensive Droplet Digital Detection (IC 3D) System comprising a system as set forth in FIGS. 17, 32 and 33.

In alternative embodiments the invention provides a multiplexed system comprising a microencapsulation droplet system integrated with a 3D particle detector as illustrated in FIGS. 1, 2, 14, 15, 17, 32, and 33.

In alternative embodiments the invention provides multiplexed portable systems comprising: an integrated micro-encapsulator and a 3D particle counting system for detecting. identifying or quantifying a target by using a method of the invention, and optionally comprising a multiplexed portable system as illustrated in FIG. 17

The details of one or more embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference for all purposes.

### DESCRIPTION OF DRAWINGS

Like reference symbols in the various drawings indicate like elements, unless otherwise stated.

FIG. 1 illustrates exemplary method of the invention comprising integrated droplet encapsulation of targets and sensing mechanisms (e.g., nucleic acid-, antibody-, enzyme- or chemical-based) followed by droplet analysis by a 3D particle detector (for example, an Integrated Comprehensive Droplet Digital Detection (IC 3D) system of the invention) for the detection and bioanalysis of low concentration targets, e.g., biological markers such as cells, biological molecules, viruses, ions and the like, and data analysis.

FIG. 2 illustrates exemplary method of the invention comprising:

FIG. 2(a) is a schematic illustration of an automated, portable device for routine bacteria detection and screening; illustrated is a droplet sample, e.g., a drop of patient blood or urine, is analyzed and the number of target bacteria in the sample is shown on a display panel within several minutes;

FIG. 2(b) is a schematic illustration of an exemplary method where a sample and a DNAzyme sensor or sensors are mixed and then encapsulated in droplets, e.g., millions of micron-sized droplets, and the DNAzyme sensors produce an instantaneous signal in the droplets that contain the bacterium, which are counted and analyzed;

FIG. 2(c) is a schematic illustration of an exemplary high throughput 3D particle counter system that permits accurate detection of single fluorescent droplet in mL volume within several minutes; see description for FIG. 17 for details of the 3D particle counter.

FIG. 3 illustrates an exemplary droplet for use in a method of the invention comprising detection and analysis of single cells and single cell markers, where the droplet has encapsulated within cell surface, intracellular and/or secreted markers, which are detected by an exemplary integrated droplet encapsulation and 3D particle detector systems of the invention.

FIG. 4 illustrates an exemplary droplet for use in a method of the invention comprising detection and analysis of cell derived particles (e.g., exosomes, microvesicles, apoptotic bodies), where the droplet has encapsulated within the droplets, and their markers can be detected by exemplary integrated droplet encapsulation and 3D particle detector systems of the invention.

FIG. 5 illustrates an exemplary droplet for use in a method of the invention comprising detection and analysis of cell-free markers including, but not limited to, nucleic acids, proteins, peptides, carbohydrates, lipids, small molecules, metal ions, etc. (which are encapsulated within the droplets), by exemplary integrated droplet encapsulation and 3D particle detector systems of the invention.

FIG. 6 illustrates an exemplary method of the invention for detecting nucleic acid mutations using padlock probes combined with nicking enzyme reaction in droplets; FIG. 6A schematically illustrates the physical process of inputting cells with probes and enzymes, their incorporation into microdroplets, followed by excitation of fluorescence and detection; and FIG. 6B schematically illustrates the molecular mechanism involving use of a so-called “padlock probe”, where ligation results in Rolling Circle Amplification (RCA), followed by nicking of cleavage site.

FIG. 7 illustrates exemplary methods of signal amplification of RCA for target detection and analysis in droplets, including use of: FIG. 7A, DNAzyme, FIG. 7B, DNA sequence replacement, and FIG. 7B, nicking enzyme.

FIG. 8 schematically illustrates systems and methods of the invention can detect low concentration targets without using droplets, e.g., using signal amplification processes such as RCA which are then stained by dye probes or nanoparticles before 3D particle counter measurement.

FIG. 9 schematically illustrates exemplary methods of the invention for detecting cellular and molecular markers using rolling circle amplification (RCE) before the 3D particle detector analysis step;

FIG. 9A illustrates exemplary of detecting cell or cell surface markers using a rolling circle amplification (RCA) process comprising components including e.g. target capture, circular DNA formation using ligation, DNA amplification via RCA, and staining or detecting process using probes including e.g., dyes or nanoparticles;

FIG. 9B illustrates exemplary of detecting a molecular target (e.g., protein) using a rolling circle amplification process comprising components including e.g. target capture, circular DNA formation using ligation, DNA amplification via RCA, and staining or detecting process using probes including e.g., dyes or nanoparticles.

FIG. 10 illustrates exemplary methods of using Real-time DNAzyme sensors in methods of the invention for selectively and rapidly detecting targets, including e.g., nucleic acids, proteins and cells, including bacterial cells and mammalian cells, e.g., as illustrated here, target E. coli in bulk;

FIG. 10(a) illustrates an exemplary mechanism of how the DNAzyme sensor generates a fluorescent signal upon interaction with the target; the target(s) produced by the bacterium binds to the inactive DNAzyme sequence (red), which undergoes a conformational change to activate the DNAzyme; the activated DNAzyme catalyzes the cleavage of the fluorogenic substrate at the ribonucleotide junction (R), leading to the separation of the fluorophore (F) and the quencher (Q) to produce a high fluorescence signal;

FIG. 10(b) graphically illustrates data from a DNAzyme sensor producing a real-time fluorescence signal in the presence of the target E. coli K12 lysates; a mutation sequence is inactive; lysates from 10,000 bacteria and 50 nM DNAzyme were mixed in a final volume in HEPES buffer and signal was recorded using a fluorescence plate reader; results are shown as mean±s.e.m (n=3);

FIG. 10(c) graphically illustrates data from a DNAzyme sensor specifically detecting E. coli strains but not non-target bacteria or mammalian cell human T cell lymphoblast CCRF-CEM and human umbilical vein endothelial cells (HUVEC); lysates from 10,000 cells and 50 nM DNAzyme were mixed in a 50 μl final volume in HEPES buffer and incubated for 30 min.; DNAzyme reaction products were analyzed by PAGE; the percentage cleavage for each reaction was derived, normalized against DNAzyme alone control and presented as “Relative fluorescence”;'

FIG. 10(d) graphically illustrates data from a DNAzyme sensors which selectively detects clinical E. coli isolates; bacteria (1000 CFU) isolated from 11 different patient samples were incubated with 100 nM DNAzyme and 1 mg ml⁻¹ lysozyme in 10% of blood for 30 minutes; fluorescence intensity was obtained using a fluorescence plate reader, normally against DNAzyme alone control (con) and presented as “Relative fluorescence”; data are obtained in a single-blind experiment;

In FIG. 10(c) and FIG. 10(d), all experiments were performed in triplicate; Data are represented as mean±s.d., n=3, ***P<0.001, ****P<0.0001, Two-tailed Student's t-test.

FIG. 11 graphically illustrates data from an exemplary method showing that DNAzyme sensors are functional and stable in diluted blood:

FIG. 11(a) graphically illustrates data showing DNAzyme sensors detecting target E. coli K12 in the bulk assay in blood diluted by sensor solution at volume ratios of 9:1, 1:1 and 1:9 corresponding to a final blood concentration of 90%, 50% and 10%, respectively; The final solution is 100 μL containing 1000 bacteria, 100 nM DNAzyme sensor and 1 mg ml⁻¹ lysozyme; the assay time is 30 min and the reaction was monitored by a fluorescence plate reader; cleaved DNAzyme sensors (by NaOH/heat) (first set of columns) and intact DNAzyme sensors (second set of columns) were included as positive and negative controls; DNAzyme sensors produced measurable fluorescence signal in the presence of E. coli in all tested blood concentrations; Data are represented as mean±s.d., n=3; this figure demonstrates DNAzyme sensors are functional and stable in blood that is diluted to different concentrations;

FIG. 11(b) graphically illustrates data showing the activity of E. coli DNAzyme sensor incubated in 30% blood at various time before adding bacteria lysates; Data are represented as mean±s.d., n=3; this figure demonstrates DNAzyme sensors are functional and stable in blood that is diluted to different concentrations.

FIG. 12 illustrates exemplary method showing DNAzyme sensors detect target bacteria E. coli in droplets:

FIG. 12(a) illustrates representative fluorescence images showing the co-localization of a single Syto17 stained bacterium and DNAzyme sensor signal after 900 s incubation time in the droplet;

FIG. 12(b) illustrates Real-time fluorescence monitoring of a single droplet that contains DNAzyme sensors and a single bacterium;

FIG. 12(c) graphically illustrates signal quantification of the fluorescence images in b);

FIG. 12(d) graphically illustrates data showing that the fluorescence intensity of droplets is directly correlated to the number of bacteria in the droplet; Minimal fluorescence signal is observed when the droplets do not contain bacteria or a mutant DNAzyme is used; 10 μm droplets are used in this Figure.

FIG. 13 illustrates fluorescence microscopy images showing E. coli DNAzyme sensors selectively detecting target bacteria in patient blood; it was also demonstrated that bacteria can be further cultured and proliferated in the droplets to amplify the signal; the left, middle and right rows represent merged, brightfield and fluorescence, respectively:

FIG. 13(a) Each droplet contains culture patient blood with 1,000˜10,000 bacteria per droplet;

FIG. 13(b) demonstrates that bacteria can be further cultured and proliferated in the droplets to amplify the signal; in this example the droplets were cultured for 5 hours;

FIG. 13(c) negative control experiment using mutant DNAzyme did not generate fluorescence in the droplets; and

FIG. 13(d) negative control experiment with healthy donor blood without bacteria did not generate fluorescence in the droplets.

FIG. 14 illustrates an exemplary device for practicing this invention comprising use of microencapsulation:

FIG. 14(a) illustrates an exemplary droplet-based microfluidic device; this exemplary device has 3 inlets; one for oil and the other two for sample, (e.g., a blood sample) and DNAzyme/bacterial lysis buffer;

FIG. 14(b) and FIG. 14(c) illustrate representative microscopy images showing uniform 30 μm droplets containing 10% blood and sensor solution generated using flow focusing, Scale bar, 200 μm, in FIG. 14(c), blood contents, especially red blood cells, are clearly visible in droplets; FIG. 14(d) illustrates droplets collected in the cuvette used for 3D particle counter experiments;

FIG. 14(e) illustrates representative fluorescence microscope images demonstrating that DNAzyme sensors (250 nM) “light up” the droplets that contain single E. coli K12 in 10% blood after 3-hour reaction; FIG. 14(e) Left panel: overlay of fluorescence and brightfield; FIG. 14(e) Right panel: fluorescence; Scale bar, 200 μm.

FIG. 15(a) illustrates a schematic diagram of an exemplary high-throughput blood micro-encapsulation device used to practice this invention; a double layer microfluidic device was designed to integrate 8 droplet generators within a single device; microfluidic devices were fabricated using Polydimethylsiloxane (PDMS) by a soft-lithography method; sensor and blood samples were introduced from the top layer and oil was injected from bottom layer; sensor and blood were merged at the middle of the top layer and they were went down through the interconnecting hole to the bottom layer, and mixed or “merged” samples were thus formed; droplets from flow-focusing structure on the bottom layer (the mixed or merged samples) were collected for particle counting.

FIG. 15(b) illustrates an image of the exemplary device described in FIG. 15A, the pictured quarter is placed to demonstrate the size of the device.

FIG. 16 graphically illustrates data from an exemplary method of the invention, where the data demonstrates that single bacteria can be detected in droplets using DNAzyme sensors and florescent droplets, and can be counted by 1D on-chip counting; SYTO 17 (red color) stained control Bacillus FIG. 16(a) or target E. coli K12 FIG. 16(b) were spiked in blood at a concentration of 10⁷ cells ml⁻¹ , which were encapsulated in a single cell manner in droplets with DNAzyme sensor (final blood content is 10% in this data); after a 3-hour reaction, droplets are counted on-chip using an exemplary confocal detection system; the (Red) spikes above 200 photon counts represent droplets that contain SYTO 17 stained cells, which are observed on both control FIG. 16(a) and target FIG. 16(b) cells; however, only the target E. coli K12 (b) produced a (green color) DNAzyme signal that is above the background (i.e., droplets that do not contain cells). At such a high initial cell concentration (10⁷ cells ml⁻¹), there are occasionally 2 bacteria (i.e., 2 (red) spikes) observed in one droplet. In these cases, the DNAzyme signal directly correlates to the number of bacteria in the droplet. FIG. 16(a) and FIG. 16(b) were performed in triplicate and a total of approximately 70,000 droplets were counted.

FIG. 16(c) graphically illustrates maximum photon counts of representative droplets that contain 0 or 1 E. coli. Black dot represents the photon count from each droplet. Box plot with an overlay of actual data is shown. Mean value is shown as red dot. n=200, ****P<0.0001, Two-tailed Student's t-test. A count is considered as a “positive hit” if it is higher than the threshold (dash lines) that is set to be the maximum photon count of empty droplets.

FIG. 16(a), FIG. 16(b) and FIG. 16(c): This set of experiments reveals that this exemplary encapsulated DNAzyme sensor system of the invention possesses zero false positive rate and minimal false negative rate (˜0.5%) using the 1D on-chip droplet counting.

FIG. 17 schematically illustrates an exemplary 3D particle counting system of the invention; as illustrated, excitation light from the laser sources (Laser1 and Laser2) is combined through the dichroic mirrors (D1 and D2) and focused on the sample (S) through an objective lens (L1); emission light collected from the same objective and transmitted through the dichroic filters is focused via a lens (L2) into a confocal pinhole (PH); the light beam is further collimated by another lens (L3) toward the detection unit; a dichroic filter (D3) splits the emission beam before reaching the emission filters (Fem) placed in front of the two photomultiplier tubes (PMT1 and PMT2); the analog signals from the PMTs (photomultiplier tubes) are converted and acquired through a card on a computer for the data analysis. 3D particle counting system of the invention are also described in further detail, below.

FIG. 18 illustrates data from an exemplary method of the invention comprising use of calibration PMTs (photomultiplier tubes) to optimize a 3D particle counter: 30 μm droplets were generated from a bacteria-spiked droplet and used to calibrate the PMTs;

FIG. 18(a) graphically illustrates raw data of fluorescent intensity traces from various PMT values (200-600).

FIG. 18(b) upper graph shows a histogram of droplet counting with various PMT values, as described in the table.

FIG. 19 illustrates an exemplary method comprising calibration RPM (revolutions per minute) to optimize 3D particle counter: 30 μm droplets were generated from bacteria spiked droplet and bright droplets were counted using 3D particle scanner:

FIG. 19(a) graphically illustrates a histogram of droplet counting in various RPM, as indicated in the table;

FIG. 19(b) is a schematic diagram to simulate the relationship between RPMs and particle counting.

FIG. 20 graphically illustrates data from an exemplary method of the invention comprising optimization of droplet size for single bacteria detection, and the data demonstrates that smaller droplets exhibited higher resolution for single bacteria detection; bacteria were spiked in healthy blood (500 bacteria/mL) and blood samples were micro-encapsulated with DNAzyme; bacteria spiked blood were encapsulated in 10, 25, or 50 μm droplets respectively.

FIG. 21 illustrates images from an exemplary method of the invention showing how droplet size (in this figures, 40 microns vs 60 microns) effects droplet detection signal; fluorescent signal is higher and produced more rapidly when droplets size are smaller because of increased effective target concentration in droplets; target: Genomic DNA extracted from MDA-MB-231 cells, Probe: TAQMAN™ probe for BRAF V600E; total of 40 cycles in this PCR reaction.

FIG. 22 graphically illustrates data from an exemplary method of the invention that normalizes actual counted particle numbers using known numbers of spiked particles (as indicated in the table) in a 3D particle counter measurement.

FIG. 23 graphically illustrates data from an exemplary method of the invention comprising single bacteria detection using 3D particle counting system (IC 3D system) along with normalization methods; the generated droplets (25 μm in diameter) containing DNAzyme sensors (250 nM) and 10% blood spiked with bacteria were collected (2 ml) in a cuvette and analyzed by a 3D particle counter:

FIG. 23(a) graphically illustrates the intensity with donor blood alone (without bacteria) mixed with DNAzyme sensors, there was no signal;

FIG. 23(b) graphically illustrates a representative bacteria sample measurement showing a typical time trace with fluorescence intensity spikes obtained from droplets containing a single E. coli K12. The temporal profile is analyzed with a pattern recognition algorithm (inset box) to extract the measurement of the concentration and/or brightness of the droplets in the sample. In this set of experiments, bacteria spiked blood was incubated with DNAzyme in droplets for 3 hours. The bacteria concentration was 1000 CFU ml⁻¹ of droplet solution;

FIG. 23(c) graphically illustrates DNAzyme reaction kinetics for quantitative bacterium detection in blood droplets measured by the exemplary 3D particle counter. A total of 1000 bacteria were spiked in this sample. Fluorescent droplets were quantified every 15 minutes using a 3D particle counter and the number of bacteria detected was plotted as y axis as function of DNAzyme reaction time. Data are represented as mean±s.d., n=3;

FIG. 23(d) graphically illustrates actual counted cell numbers using the Integrated Comprehensive Droplet Digital Detection (IC 3D) (y axis) vs. a broad range of spiked bacteria concentration (i.e., “theoretical number of bacteria”) (x axis: numbers of bacteria per milliliter of collected droplet solution). Y=0.95×. R²=0.999. Standard curve was built using droplets containing FITC or droplets containing fluorescent DNAzyme sensor after reacting with bacteria. To precisely achieve extremely low bacterial concentration (1-50 cells ml⁻¹), bacteria were collected and spiked into blood using a microinjector system prior to encapsulation. Bacteria spiked blood was incubated with DNAzyme in droplets for 3 hours in this set of experiments. Data are represented as mean±s.d, n=3. Note that the small size of the error bars for concentrations of 100, 1,000 and 10,000 cells ml⁻¹.

FIG. 24 graphically illustrates selective detection of clinical E. coli isolates (using E. coli-specific probes) using the exemplary Integrated Comprehensive Droplet Digital Detection (IC 3D)system of the invention; representative 3D particle counter data demonstrate that only target E. coli isolate among 11 different bacterial isolates (as indicated in the figure) generate typical fluorescence intensity spikes in a single-blind experiment. The total number of counted cells in each sample is shown in the boxes in top left corner. E. coli K12 spiked blood was used as a positive control.

FIG. 25 in table form summarizes the major performance specifications of the exemplary IC 3D system and method of the invention as compared to PCR tests (e.g., FILMARRAY™, BioFire Diagnostics, Salt Lake City, Utah) that were approved by FDA for bacterial detection. The exemplary IC 3D of the invention provides absolute quantification of target bacteria in blood at a broad range of concentrations from 1 to 10,000 bacteria/mL within approximately 1.5-4 hours (droplet generation (<40 min)+DNAzyme sensor reaction (approximately 45 min for “yes or no” and approximately 3.5 hours for absolute quantitation)+3D particle counting (3-10 min)+data processing (5 min)) with single-cell sensitivity and an exceptional limit of detection (LOD) in the single digit regime.

FIG. 26 illustrates detection of beta-lactamase producing bacteria using commercially available fluorogenic substrate:

FIG. 26(a) graphically illustrates data showing a test in bulk using cell lysates. Bacteria lysates were mixed with 2 μM fluorogenic substrate in a 50 μl final volume in PBS buffer and incubated for 20 min. Reaction mixtures were analyzed using a fluorescence plate reader;

FIG. 26(b) graphically illustrates data showing that single beta-lactamase-producing bacteria can be detected in droplets using fluorescent microscope and Integrated Comprehensive Droplet Digital Detection (IC 3D)systems of the invention. The generated droplets (20 μm in diameter) containing 2.5 μM fluorogenic substrate and single bacteria (Isolate 1 or 7, as indicated in FIG. 26(a)) were collected in a cuvette and incubated. After overnight incubation at room temperature, droplets were analyzed by particle counter (FIG. 26(b, top panels) and microscope (FIG. 26(b, bottom panels). The temporal profile is analyzed with a pattern recognition algorithm (top panels).

FIG. 27 illustrates images of the detection of BRAF V600E mutation using droplet digital PCR. Genomic DNA was isolated from FIG. 27(a) HCT116 (wild-type BRAF, negative control, lacks the mutation) and FIG. 27(b) COLO 205 cells (have the BRAF V600E mutation). Isolated genomic DNA was encapsulated in 20 μm droplets and real-time quantitative PCR was performed to identify BRAF V600E mutation.

FIG. 28 demonstrates effective PCR amplification of nucleic acids in droplets comprising blood content using a process of the invention. The PCR primer amplification target was a 56 nucleotide (nt) long synthetic DNA template; negative controls were without target. The figure illustrates a representative gel image showing the detection of the synthetic target DNA in 20% blood. PCR was performed in 30 or 40 cycles. Negative controls were the same reaction without target DNA.

FIG. 29 graphically illustrates data from an exemplary system/method of the invention using a 3D particle counter to detect cells in blood:

FIG. 29(a) illustrates detection of spiked cancer cells in blood using a 3D particle scanner system of the invention;

FIG. 29(b) illustrates flow cytometry used as a control;

For FIG. 29, lymphocyte separation centrifugation method was used to isolate WBC, and cancer cells (MDA-MB-231) were spiked into whole blood; cells are stained with cell tracker Green or labeled with RFP.

FIG. 30 graphically illustrates data from an exemplary method of the invention using the exemplary IC 3D of the invention to detect Let-7a quantification in plasma:

FIG. 30(a) graphically illustrates a representative time trace with fluorescence intensity profiles of droplets obtained from blank (left panel), Let-7a (middle panel) and Let-7b (right panel); only the target Let-7a group generates fluorescence intensity spikes, which demonstrates the specificity of the IC 3D assay of the invention; the miRNA concentration is 10 fM in exosome-depleted plasma before encapsulation;

FIG. 30(b) graphically illustrates actual counted Let-7a number using the exemplary IC 3D of the invention (y axis) vs. spiked Let-7a concentration (x axis); error bar is based on triplicate experiments; mean±S.D;

FIG. 30(c) graphically illustrates data from RT-qPCR for Let-7a detection in plasma (after miRNA purification and reverse transcription); Error bar is based on triplicate experiments; Mean±s.e.m;

FIG. 30(d) graphically illustrates data from Let-7a concentration quantification in 3 healthy donor plasma samples and 3 colon cancer patient plasma samples detected by RT-qPCR and the exemplary Integrated Comprehensive Droplet Digital Detection (IC 3D)of the invention; Error bar is based on triplicate experiments. Mean±s.e.m. P value<0.05 (Student T test).

FIG. 31 illustrates images of droplet microfluidics-based single cell engineering using exemplary systems of this invention, where single MCF7 cells were encapsulated with transfection reagent containing GFP expression vector using a microfluidic device:

FIG. 31(a): images demonstrating that after encapsulation, droplet stability was confirmed after 6 hours; FIG. 31(b), images demonstrating that after transfection within droplet, GFP protein was expressed in the cells (see right-hand panel).

FIG. 32 schematically illustrates an exemplary portable system of the invention comprising: an integrated micro-encapsulator and a 3D particle counting system of the invention; FIG. 17 describes the 3D particle counter in detail. Integrated Comprehensive Droplet Digital Detection (IC 3D)systems of the invention can be connected by remote devices, e.g., with a smart phone or an iPad through Bluetooth. The remote, e.g., smart phone, interface can therefore be used to operate the system, collect and analyze data, and send or deliver data to the doctors, patients and health care providers, etc.

FIG. 33 schematically illustrates a system of the invention comprising an Integrated Comprehensive Droplet Digital Detection (IC 3D) automated and integrated device and system of the invention, which in alternative embodiments are portable, and can be high throughput droplet generation systems::

FIG. 33(a) illustrates a 70-channel Telos system from Dolomite that can be used to practice the invention;

FIG. 33(b) illustrates a 256 channel cartridge system can be used to practice the invention that can encapsulate a 3 mL sample into droplets of 30 μm diameter in less than 15 min;

FIG. 33(c) illustrates an ISS QUANTA 3D particle detector that is an automated, portable and multiplex system;

FIG. 33(d) schematically illustrates a “Sample to result” measurement using this exemplary IC 3D system of the invention.

FIG. 34 illustrates an exemplary method of the invention for facile cancer diagnostics using in vitro evolution of DNAzyme sensors:

FIG. 34(a) illustrates an exemplary system of the invention comprising use of a mix-and-read, DNAzyme sensor cancer diagnostic and its applications for routine cancer screening, early stage cancer diagnosis and prognosis, monitoring disease progression and recurrence, and monitoring drug effectiveness and safety:

FIG. 34(b) schematically illustrates and exemplary mechanism of a DNAzyme sensor that can be used to practice this invention: it generates fluorescent signal upon interaction with the target (F is a Fluorescein-dT. R is ribonucleotide and Q denotes a dabcyl-dT):

FIG. 34(c) is a schematic illustration of an in vitro selection process that can be used to practice this invention: First, the random DNA library is ligated to the substrate and incubated with normal serum to remove any non-specific sequences from the library pool; the un-cleaved sequences are purified and applied to the positive selection using the cancer serum; the cleaved molecules by the cancer serum are purified and amplified by PCR; and, after purification, the population is ligated to the substrate and applied to the next round of selection.

FIG. 35 describes an exemplary library (the so-called “DzL”) and sequences to generate DNAzyme sensors for cancer diagnosis: DzL is the library wherein N denotes the random nucleotides; FSS, DzL-FSS-LT, DzL-FP, DzL-RP1 and DzL-RP2 are substrate, ligation template, forward primer, reverse primer1 and reverse primer 2 respectively.

FIG. 36 illustrates an exemplary method for monitoring DNAzyme evolution and selection using denaturing polyacrylamide gel electrophoresis (dPAGE) in practicing the invention:

FIG. 36(a) dPAGE image of the negative selection of round 1 of DNAzyme evolution;

FIG. 36(b) dPAGE image of positive selection of round 1 of DNAzyme evolution, the boxed region was excised, and the DNA was eluted for PCR amplification; M=marker (made by heating the ligated library with 0.25 M NaOH at 90 C), RM=Reaction mixture.

FIG. 37 illustrates an exemplary method for monitoring DNAzyme evolution and selection using denaturing polyacrylamide gel electrophoresis (dPAGE) in practicing the invention: dPAGE images of positive selection of round 7 FIG. 37(a) and round 11 FIG. 37(b) respectively; the boxed region was excised, and the DNA was eluted for PCR amplification. M=marker (made by heating the ligated library with 0.25 M NaOH at 90 C), SB=selection buffer. MNS=mixed normal serum, MCS=mixed cancer serum; the boxed regions were excised and the DNA was eluted for PCR.

FIG. 38 illustrates use of a selected set of DNAzyme sequences (LCS19-1, 19-2, 19-3 and 19-4, see below) for cancer diagnosis obtained using in vitro evolution; these DNAzyme sequences were tested using PAGE; the cleavage performance was justified based on the cleaved bottom band intensity; the target or activator of a given DNAzyme can be a protein, a nucleic acid, a small molecule, or metal ions, or a combination etc:

LCS19-1:
(SEQ ID NO: 7)
GTCAGCCATGAGTAAGCGGGAAGCGTATAGCCTAAATGGGATGGACGTAC
CAACGAGGATCTGTCGTCTCACTC
LCS19-2:
(SEQ ID NO: 8)
GTCAGCCATGAGTAAGCATCAGCAGCCCACTAGATAAGTGGAGGGAAAGT
CTGTACAGATCTGTCGTCTCACTC
LCS19-3:
(SEQ ID NO: 9)
GTCAGCCATGAGTAAGCGGGGAGCGAGTCATGAGAAAATCGCGGGGAAGC
ACAGGGTGATCTGTCGTCTCACTC
LCS19-4:
(SEQ ID NO: 10)
GTCAGCCATGAGTAAGCAATTGATCGTGGAACCAGACGAATAAACCACAG
GATTTAGGATCTGTCGTCTCACTC

FIG. 39(a) illustrates an exemplary method to prepare one type of DNA/one bead using combinatorial DNA synthesis on microparticles (a); FIG. 39B illustrates an exemplary method to construct a DNA-bead library using one type of DNA/one bead method; functional sites including primer binding sites and restriction sites can be incorporated for subsequent PCR, sequencing, transcription and translation and strand release from bead purposes; the illustrated “target sequence” is SEQ ID NO:11.

FIG. 40 illustrates an exemplary method of droplet library generation, device design, manipulation and applications of the invention: Exemplary droplet manipulation includes e.g., droplet merging, splitting, incubation, reinjection, imaging, analysis and sorting: Exemplary droplet assays include e.g., PCR, transcription, translation, and a variety of biological and chemical reactions and interactions; Exemplary droplet library-based screening is used to, e.g., study biological interactions, developing diagnostics and therapeutics.

FIG. 41 illustrates an exemplary method of the invention using a DNA-bead-droplet library for screening. Each droplet contain single bead that is immobilized with multiple DNA of same sequences. PCR can be performed to amplify DNA and produce free DNA library in the droplets. Beads can be removed and/or used by themselves for target binding and sorting. Droplets can be distributed onto microwell chip for further processes including purification, target binding, reactions, screening, sequencing, and transferring to a chip to fabricate nucleic acid or protein arrays.

FIG. 42 illustrates an exemplary method of the invention using a DNA droplet library for biomarker screening from patient blood, cancer cells, and etc. Molecules encapsulated in the droplet and/or droplets can be barcoded which permits subsequent analysis and identification. Droplet assay can be coupled with chip-based or array based arrays for high throughput analysis and biomarker identification.

FIG. 43 illustrates images demonstrating the successful single bead encapsulation in droplets using an exemplary system of this invention; beads used in this example were 6 μm fluorescent, magnetic iron oxide crystals obtained from Bangs Laboratories, Inc. (Fishers, Ind.).

FIG. 44 illustrates an exemplary method and device of the invention to manipulate or process a droplet or a bead library: the magnetic bead can be relocated in the droplet using a magnet; a droplet can be split into two droplets using a micro-blade, and this gives rise to DNA droplet libraries with or without bead in the droplet, each of which can be used for subsequent screening or biomarker discovery.

FIG. 45 illustrates an exemplary method for practicing this invention using a DNA-bead and DNA-bead droplet library or FACS based screening for molecules that bind to e.g., cancer cells or cell membrane markers; in the schematic illustration of an exemplary protocol or system of this invention: a DNA-bead library is first mixed with target sample e.g., purified targets such as proteins or complex samples such as blood, cells, or tissues; the bound target/bead complex can be sorted by magnetic sorting and/or, after staining targets with a dye, antibody or other probes, by FACS; the bound target/bead complex can be dissociated using e.g., buffer at high or low pH, urea, EDTA, etc.; the dissociated targets can be processed and analyzed further for identification and characterization; the screening can be performed both in a single round or multiple rounds; and, a negative selection using non-target or control samples can be integrated into the selection process to improve binding specificity of binders.

FIG. 46 illustrates an exemplary method of the invention using a droplet library to screen molecules that can e.g., modulate protein-protein interactions or enzyme reactions.

FIG. 47 illustrates a schematic of an exemplary system of the invention using an aptamer inhibitor-DNA-enzyme (IDE), or aptamer-IDE, system. Initially, the enzyme is in an inactive state because its inhibitor (which can be, e.g., a small molecule inhibitor), by binding to the enzyme inhibits or allosterically modifies enzyme activity, e.g., by binding in or occupying the active site and/or an allosteric site. The inhibitor is tethered to the enzyme by an aptamer-containing nucleic acid (e.g., DNA, or artificial or synthetic nucleic acid) sequence. When the target molecule is added, the aptamer constructs tertiary structure around the target molecule, thereby displacing the inhibitor from the enzyme's active site or allosteric site (for example, when the aptamer-IDE binds to its target, its conformation changes, thus releasing the inhibitor from the enzyme to “release inhibition”, or activate, the enzyme). The enzyme is then activated, or is freed, and it can then enzymatically generate a detectable signal, e.g., can generate a fluorescence, e.g., can enzymatically turnover multiple copies of a detectable substrate, e.g., a fluorogenic substrate.

FIG. 48 illustrates an exemplary system and method of the inventions for generating aptamer-containing (e.g., aptamer-IDE-containing) droplets, the method given the name: ENcapsulated ScreeNing of Aptamers by Reporter Amplification (ENSNARA), as described in detail, below.

FIG. 49 illustrates exemplary fluorescent microscopy images showing single enzyme detection in droplets: FIG. 49a) illustrates beta-galactosidase in 30 μm droplets with its fluorogenic substrate; and FIG. 49b) is the negative control with encapsulated substrate alone without enzyme.

FIG. 50 illustrates an exemplary system of the invention for single nucleic acid molecule amplification in a droplet using EXPonential Amplification Reaction (EXPAR) which can be used in an ENSNARA system of the invention:

FIG. 50a illustrates an exemplary mechanism for an EXPonential Amplification Reaction (EXPAR) reaction: DNA template is designed with two repeated sequences in both 3′ and 5′ termini separated by a nicking site (e.g., nicking endonuclease Nt.BstNBI). Both repeating units in each side are complementary to the target nucleic acid strands (i.e. 10 “initiator” strands); therefore, the target strand can hybridize with the template and then is extended along the template by DNA polymerase (e.g. Vent (exo-)) to form double stranded DNA (dsDNA); nicking enzyme recognizes the nicking site on the dsDNA and cleaves the newly synthesized strand; after cleavage, the upstream sequence serves a primer to be extended by DNA polymerase and replace the downstream sequence; since the replaced downstream sequence is the same DNA sequence as the target nucleic acid, it serves as free primer to start a new reaction with a free template; a dsDNA-binding dye such as EvaGreen mixed in reaction mixture binds to the amplified sequences to generate fluorescent signal that can be monitored in a real-time fashion; and,

FIG. 50b illustrates fluorescent microscope images demonstrating an exemplary use of EXPAR for single synthetic nucleic acid detection in a droplet; the fluorescent microscope images monitor fluorescence signals from droplets over time (bottom row); the bulk concentration of spiked synthetic nucleic acid target before encapsulation was 10 fM which translates to 0 or 1 molecule per droplets after encapsulation; control droplets that do not contain target nucleic acid did not produce fluorescence in studied time window; the images at time points <40 min are not shown because there are few fluorescent droplets. Scale bar: 200 μm.

FIG. 51 schematically illustrates an exemplary “allosteric” IDE system of the invention comprising: a reporter enzyme conjugated to a multi-domain DNA sequence; left panel shows the enzyme of the so-called “IDE” complex inhibited by contact with inhibitor, and right panel shows that addition of a 26-mer complementary sequence (D¹) to the alpha-loop releases the inhibitor, thereby activating the enzyme.

### DETAILED DESCRIPTION

The invention provides powerful, high throughput analytical platforms which can monitor a liquid sample (e.g., whole human blood, serum, saline or water, or any environmental sample) to detect biological, physiological and pathological markers with extremely high sensitivity (e. g., a single molecule or a single cell), and methods for making and using them. In alternative embodiments, system integrates novel sensor, e.g., biosensor, technology and a high throughput particle or droplet microfluidics platform. In alternative embodiments, the biosensors are short oligonucleotides, antibodies, peptides or other sensing elements that are engineered to specifically react with the targets, leading to a rapid fluorescence signal. In alternative embodiments, signals can be amplified using standard, conventional assays including PCR, rolling circular amplification, proximity ligation assays and EXPonential Amplification Reaction (EXPAR).

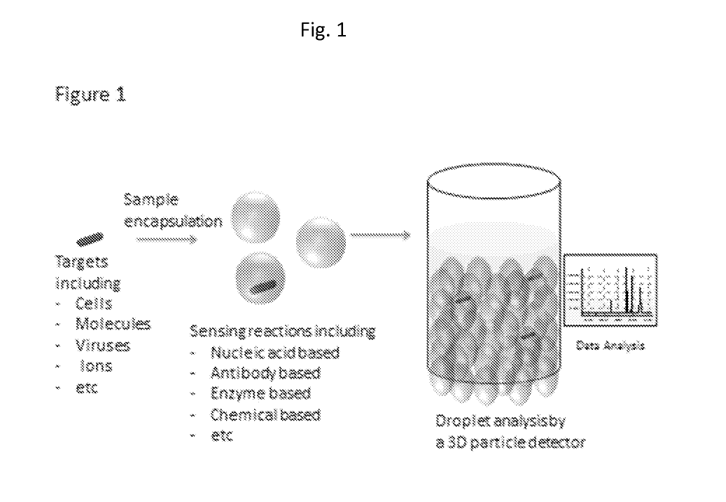

In alternative embodiments, exemplary platforms or systems of the invention enable rapid and sensitive detecting of a small molecule, or a biological, a physiological or a pathological marker, or a single molecule or a single cell using a microencapsulation droplet system integrated with a 3D particle detector (termed “Integrated Comprehensive Droplet Digital Detection (IC 3D)”), where the core concept of the integrated droplet encapsulation and 3D particle detector for the detection and bioanalysis of: low concentration biological markers, or for the detection and diagnosis of complex diseases including infectious diseases, cancer, diabetes, Alzheimer's disease, and the like, is schematically illustrated in FIGS. 1, 2, 3, 4, 5, 6, 7, 8 and 9.

In alternative embodiments, the invention provides high throughput, multiplexed systems or methods for detecting a small molecule, or a biological, a physiological or a pathological marker, or a single molecule or a single cell using a particle or a droplet-based microfluidics system integrated with use of a sensor, e.g., a nucleic acid such as a DNAzyme. In alternative embodiments, the sensors, e.g., the DNAzymes, used to practice this invention are capable of specifically binding to a target molecule or a specific cell. In alternative embodiments, the target molecule or cell comprises a biological, physiological or pathological marker, or a single molecule or a single cell.

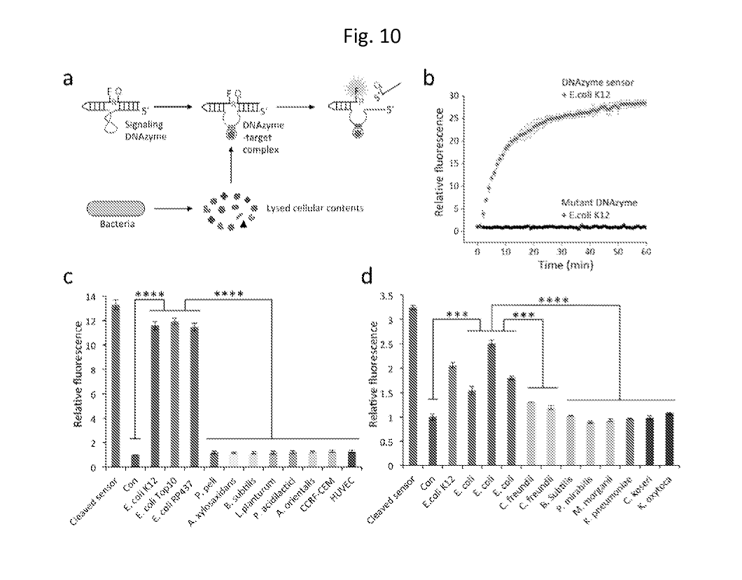

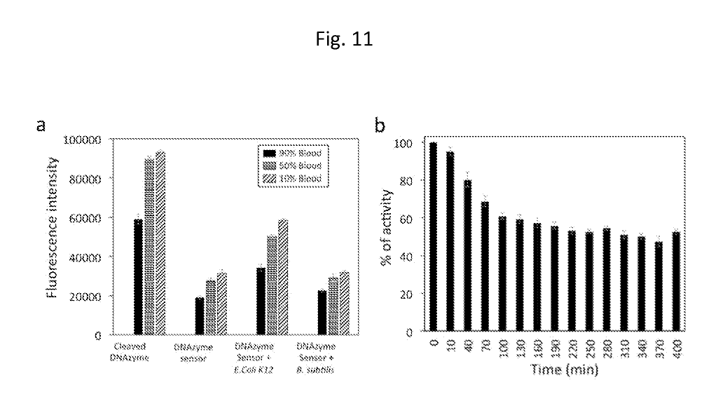

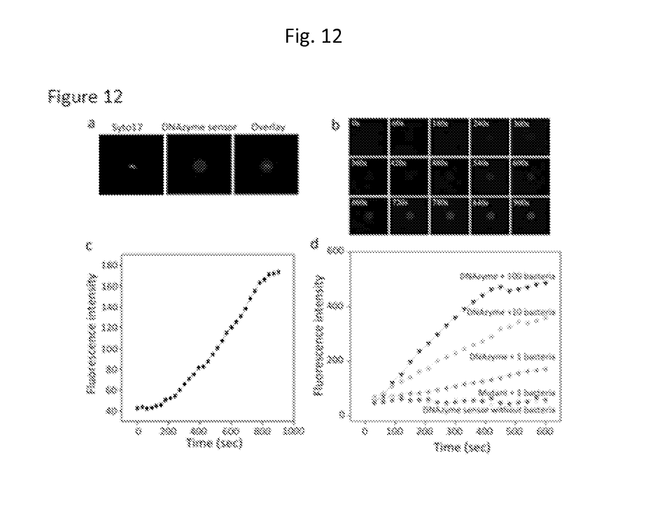

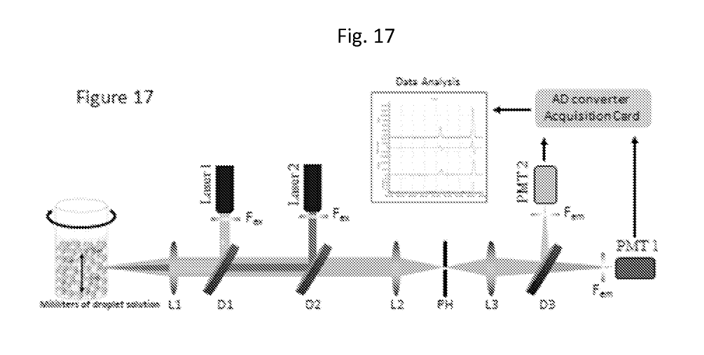

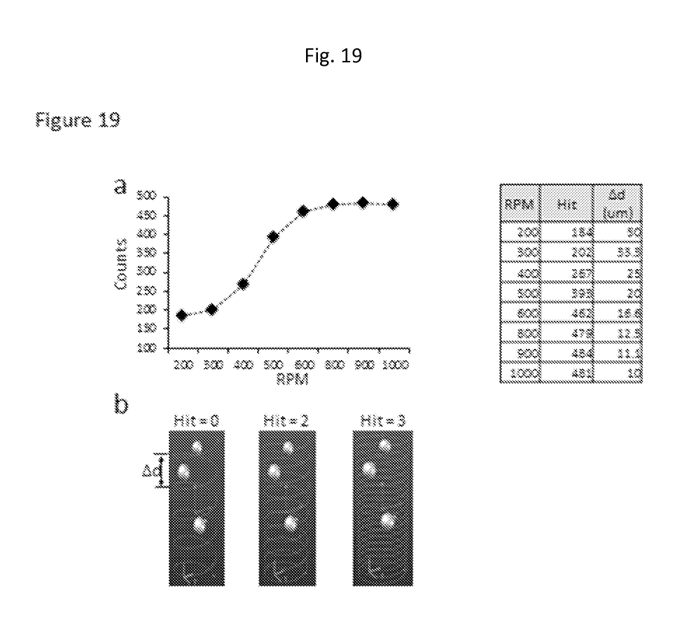

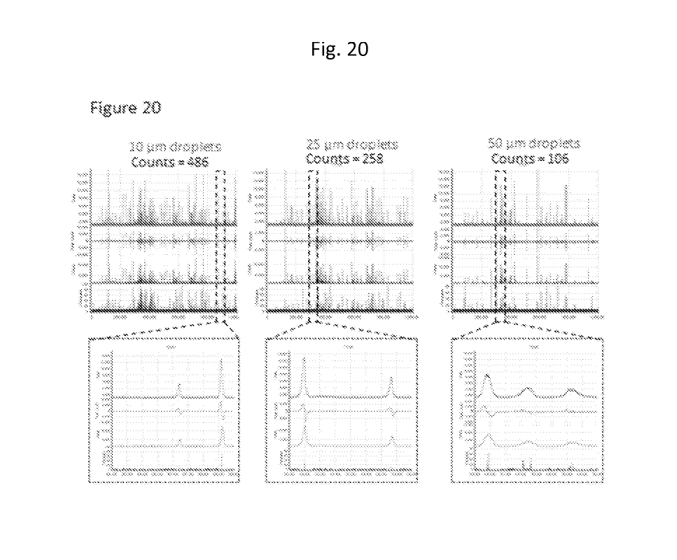

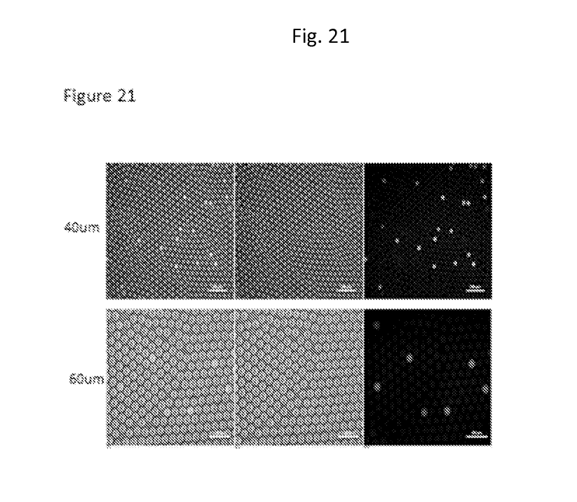

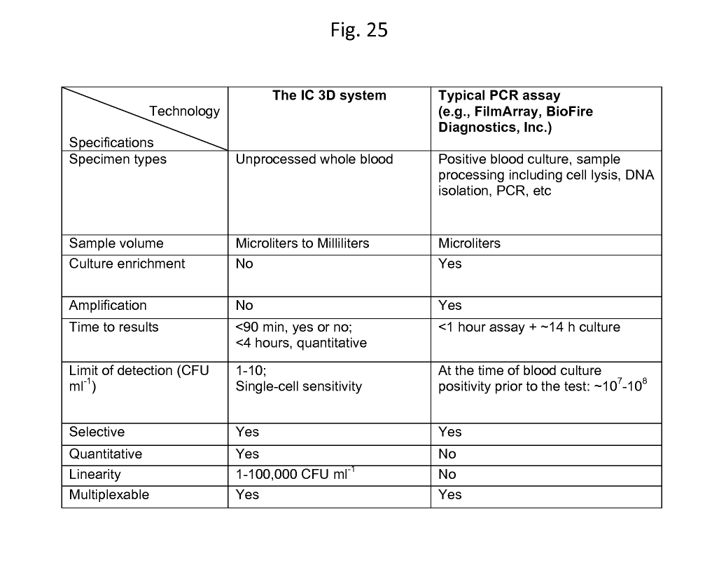

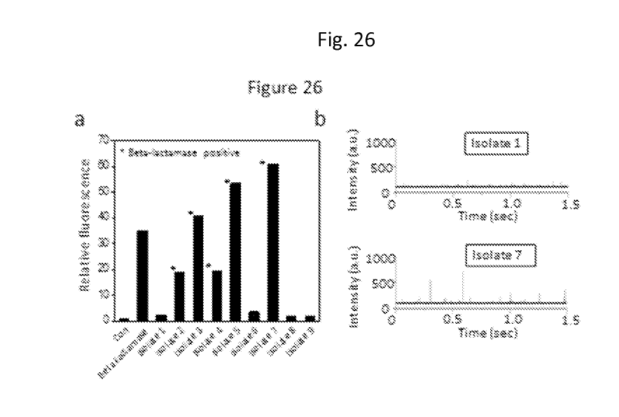

We demonstrated the effectiveness of an exemplary system of the invention comprising droplet microfluidics system integrated with a sensor, e.g., a DNAzyme. DNAzymes, also called deoxyribozymes or DNA enzymes or catalytic DNA, are DNA molecules that have the ability to perform a chemical reaction or catalyze a reaction. In practicing these exemplary systems and methods of the invention, the sensor, or the DNAzyme sensor (FIGS. 10a and b), can detect bacteria in single cell manner within few hours from whole blood; also, single bacteria detection has been also achieved within 15 minutes in buffer (FIGS. 10c and d; FIG. 11). In alternative embodiments, the compartmentalization of human blood in droplets (which can be between about 1 to 300 um, or 10 to 100 μm, in diameter) increased significantly the assay sensitivity, reduced background, and decreased assay time by increasing the effective concentration of target species, and by preventing the diffusion of targets and sensors from the tiny space of the droplet (as illustrated, e.g., in FIGS. 12, 13, 14, 15 and 16). In alternative embodiments, the integration of a 3D particle counter (“Integrated Comprehensive Droplet Digital Detection (IC 3D)” enables selective detection of bacteria directly from milliliters of whole blood at single-cell sensitivity in a one-step, culture- and amplification-free process within 1.5-4 hours (see, e.g., FIGS. 17, 18, 19, 20, 21, 22, 23, 24 and 25). In alternative embodiments, microencapsulated systems of the invention comprise use of fluorogenic substrates for enzyme markers including beta-lactamases (e.g., a carbapenemase) for the detection of extended spectrum beta-lactamase (ESBL)-producing Enterobacteriaceae and carbapenem-resistant Enterobacteriaceae (CRE), TB and other antimicrobial resistant pathogens (see, e.g., FIG. 26).

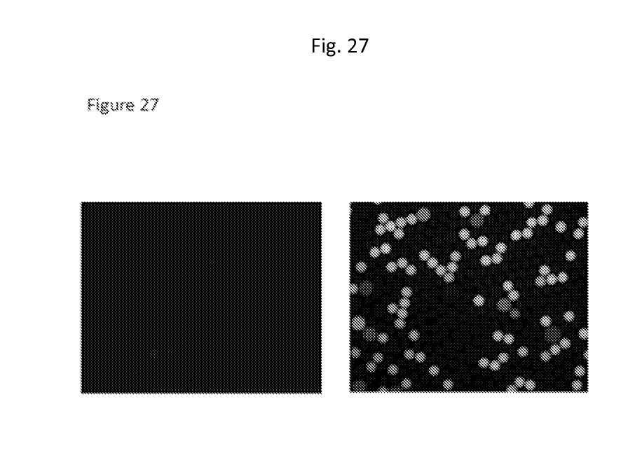

In alternative embodiments, systems of the invention can be used to detect rare circulating tumor cells in blood. In alternative embodiments, systems of the invention can specifically assess gene expression, point mutations, miRNAs and SNPs using droplet-PCR, droplet RT-PCR or droplet-EXPonential Amplification Reaction (EXPAR) (see, e.g., FIGS. 27, 28, 29 and 30). In alternative embodiments, systems of the invention can specifically assess secreted and intracellular protein markers using e.g., real-time fluorescent sensors. In alternative embodiments, exemplary platforms or systems of the invention can be used for cell isolation and sorting, and for study of tumor heterogeneity, single cell-cell interactions (stem cell-cancer-immune cell), cancer stem cells, evolution, cell-drug interaction and drug resistance. In alternative embodiments, the invention provides study, monitor, and track single transplanted cells including, e.g., stem cell and cancer stem cells. In alternative embodiments, exemplary platforms or systems of the invention can be used to detect circulating melanoma cells in blood, for example, taking advantage of the intrinsic signals from these cells, optionally without using any sensors.

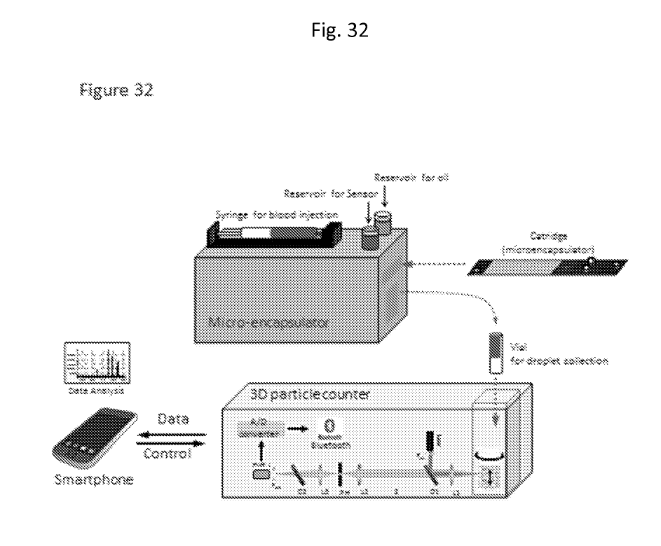

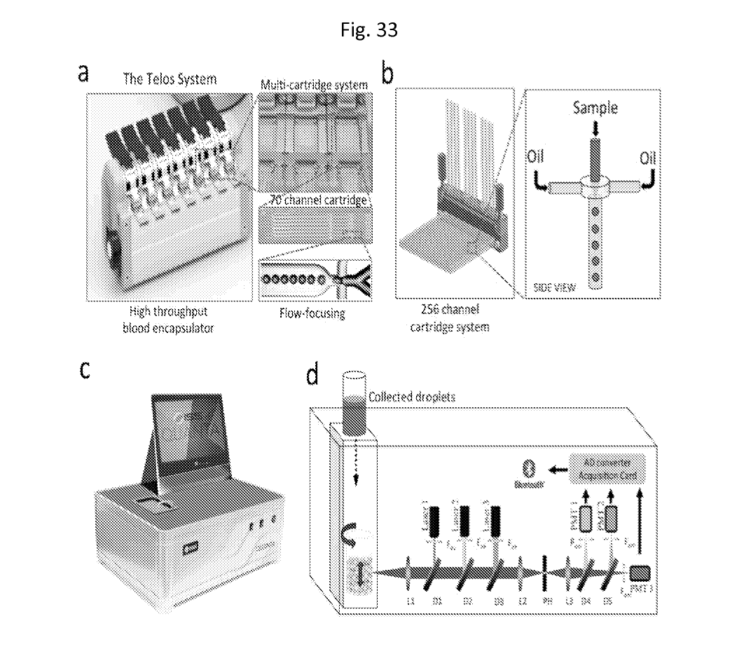

In alternative embodiments, the invention provides systems comprising Integrated Comprehensive Droplet Digital Detection (IC 3D) (e.g., as illustrated in FIGS. 32 and 33).

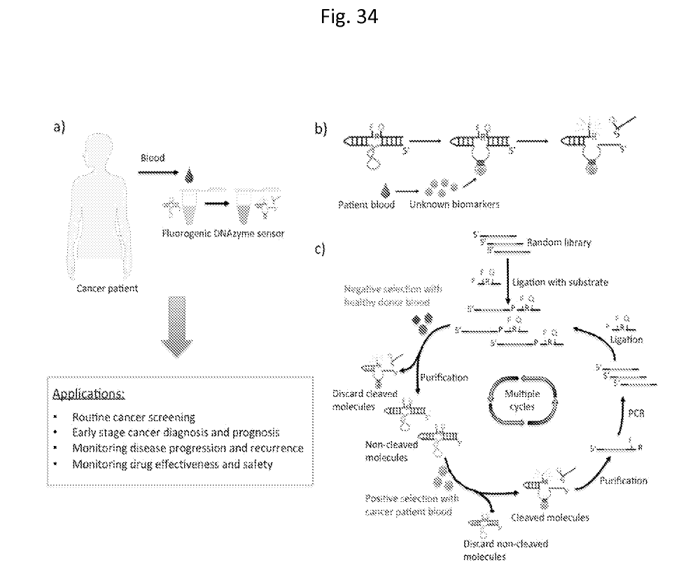

In alternative embodiments, exemplary platforms or systems of the invention comprise use of multiple rounds of enrichment using, e.g., disease and/or normal samples as positive and negative selection targets, respectively (see, e.g., FIG. 34). This embodiment can be used to identify sensors, e.g., DNA sensors, that specifically recognize a vital (or a unique panel of) molecular signature(s), e.g., SNPs, deletions, transpositions, proteins and the like, that discriminate the disease sample from normal samples. In alternative embodiments the target samples in the selection are complex systems including blood, serum, or tissue samples.

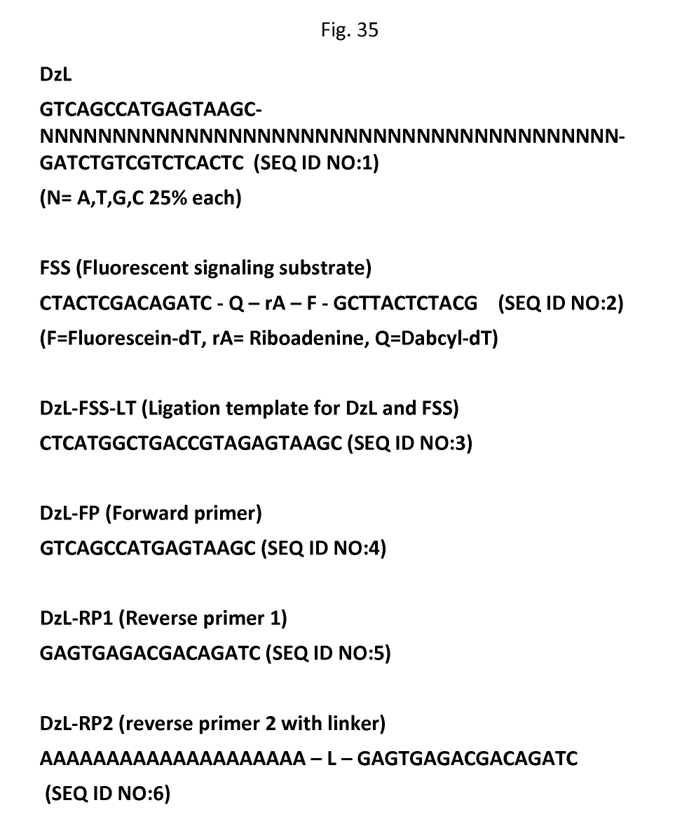

We have completed exemplary DNAzyme screening processes of the invention for lung cancer, and obtained several DNAzyme sensors (as illustrated, e.g., in FIGS. 35, 36, 37 and 38). In alternative embodiments, these DNAzymes are integrated with droplet microfluidics for cancer detection. In alternative embodiments, exemplary platforms or systems of the invention use a strategy to obtain molecular and cellular signaling aptamers using in vitro selection that directly couples to a downstream signaling pathway. In alternative embodiments, exemplary DNAzyme screening processes identifies aptamers that specifically modulate stem cell differentiation into a particular lineage.

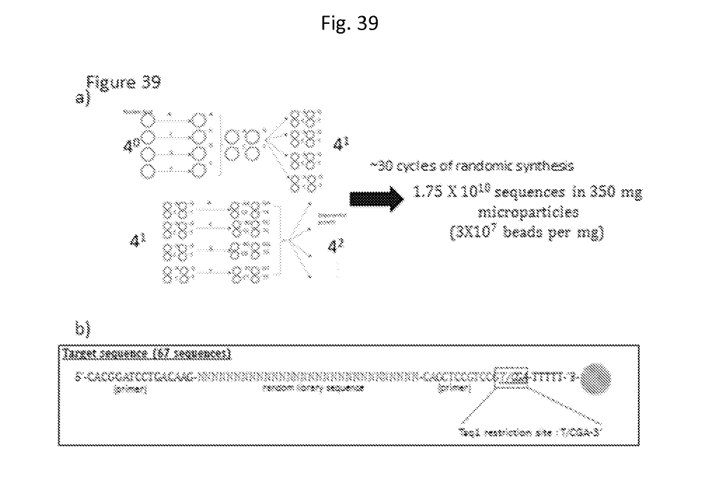

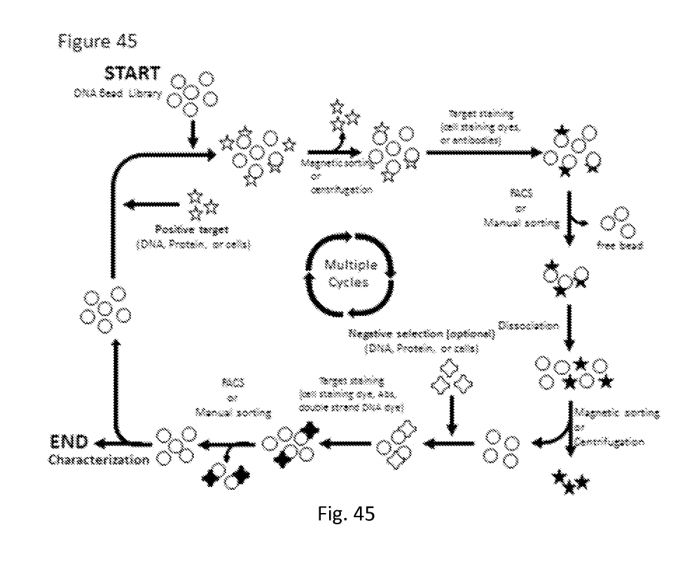

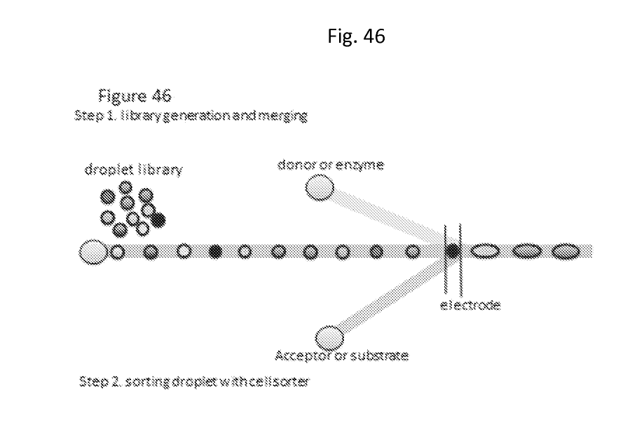

In alternative embodiments, exemplary platforms or systems of the invention can exploit powerful in vitro selection to generate reliable, nucleic acid binders, agonist or antagonist or DNA sensors and diagnostics for complex diseases including cancer, diabetes Alzheimer's disease, and the like (as illustrated e.g., in FIGS. 39, 40, 41, 42, 43, 44, 45, and 46).

In alternative embodiments, the invention's droplet microfluidic systems are significantly more effective, more sensitive, easier to make, and more tunable compared to existing ones to monitor biomarkers for diagnostics and prognostics. In alternative embodiments, the droplet libraries generated by methods and systems of the invention can significantly increase the chance to find drugs candidates and new biomarkers with small sample amount and also can reduce the screening time.

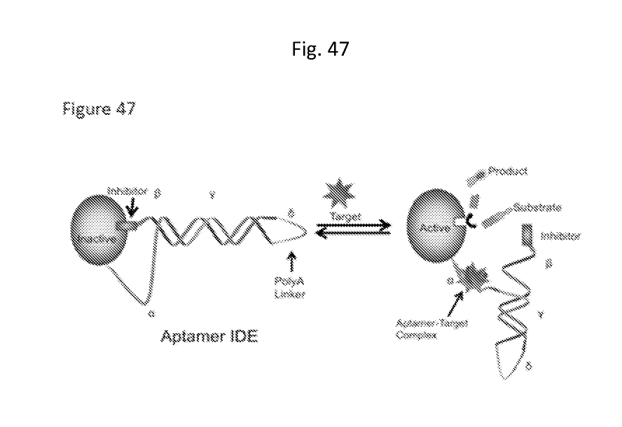

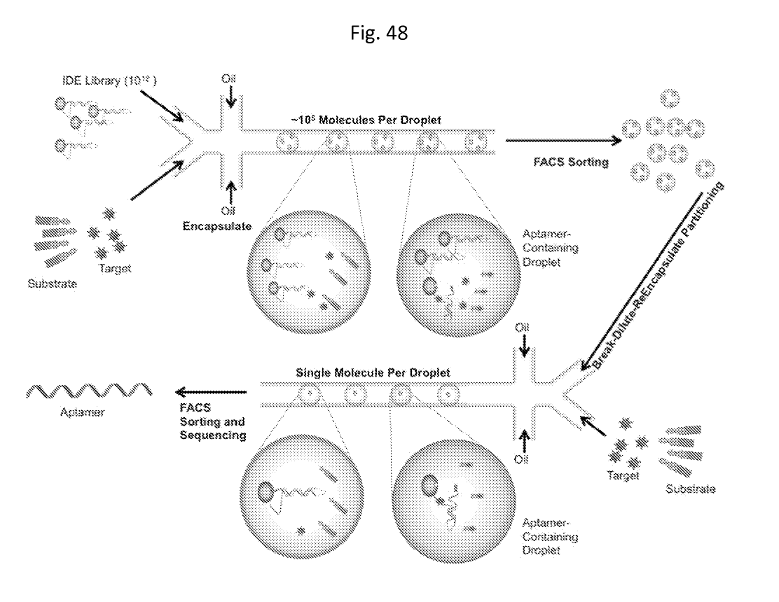

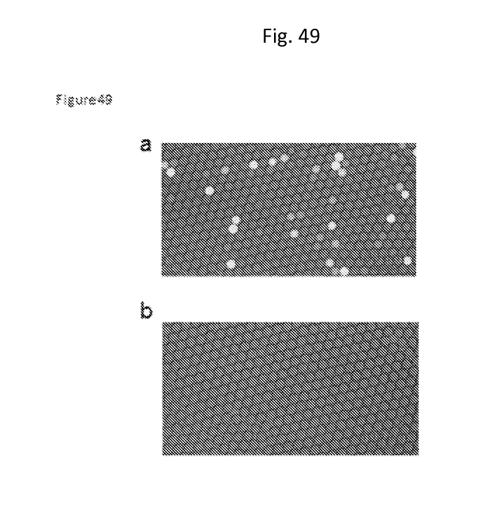

In alternative embodiments, exemplary platforms or systems of the invention includes a method called ENcapsulated ScreeNing of Aptamers by Reporter Amplification (ENSNARA) for identification of aptamers by employing allosteric control over a reporter enzyme or an enzyme system in the droplet; e.g., as illustrated in FIGS. 47 and 48, and as described in detail in Example 8, below. In alternative embodiments, an exemplary ENSNARA method of the invention comprises first providing an initial aqueous mixture comprising an aptamer inhibitor-DNA-enzyme (IDE)library or plurality of aptamer-IDEs (which can be greater than 10¹² molecules, as illustrated in FIG. 48), a fluorogenic substrate (e.g., a direct substrate for the enzyme) and a target molecule (that is bound, e.g., specifically bound, to by the aptamer-IDE), and these are pumped through an oil stream. As the immiscible fluids come into contact, the aqueous components are compartmentalized into millions of picoliter sized droplets.

For this exemplary ENSNARA protocol, in the first stage there are 10⁶ IDE per drop. A sorter (for example, a FACS, as illustrated in the figure) directs any fluorescent droplets into a separate channel, where they are lysed, diluted, and re-encapsulated at a concentration of 1 aptamer inhibitor-DNA-enzyme (IDE) per drop, and supplemented with substrate and target molecule (substrate and target molecule are added to, or incorporated within, the re-encapsulated 1 IDE per drop microdroplets). The aptamer-containing droplets that produce a fluorescent signal are then collected, and optionally the aptamer can be sequenced.

### Microfluidic Systems and Using and Transporting Microdroplets

In alternative embodiments, the systems and methods of the invention can use any form or variation of microfluidic systems for making, using and/or transporting microdroplets to practice this invention.

For example, a microfluidic transport system for transporting microdroplets in three spatial dimensions can be used as described e.g., in U.S. patent app. pub. No. 2013/0213812. In alternative embodiments, the systems and methods of the invention can use microdroplet-manipulating devices coupled to a movement and placement device as described e.g., in U.S. patent app. pub. No. 20130149710, which also described PCR reactions in the microdroplets. U.S. patent app. pub. No. 20130139477 describes use of microdroplets as “microreactors” for controlled processing of the contents, wherein very small amounts of material are encapsulated in a microdroplet in a quantized amount. U.S. patent app. pub. No. 20130130919 describes a microdroplet-based method for sequencing large polynucleotide templates. Microdroplets can be made e.g., by an apparatus as described in U.S. patent app. pub. No. 20130129581.

In alternative embodiments, the systems and methods of the invention can use microdroplet-manipulating devices as described e.g., in: U.S. Pat. No. 8,529,026, describing devices for passively periodically perturbing a flow field within a microfluidic device to cause regular droplet formation at high speed; or U.S. Pat. No. 8,528,589, describing methods for assessing one or more predetermined characteristics or properties of a microfluidic droplet within a microfluidic channel, and regulating one or more fluid flow rates within that channel to selectively alter the predetermined microdroplet characteristic or property using a feedback control; or U.S. Pat. No. 8,492,168, describing droplet-based affinity assays, e.g., detecting a target analyte in a sample by combining affinity-based assay reagents on a droplet microactuator with a sample/target analyte to generate a signal indicative of the presence, absence and/or quantity of analyte; or U.S. Pat. No. 8,470,606, describing methods of circulating magnetically responsive beads within a droplet in a droplet actuator, and methods for splitting droplets; or, U.S. Pat. No. 8,524,457, describing methods for screening specific affinity molecules to target molecules using a homogeneous non-competitive assays using e.g., microdroplets created e.g., using micro- or nanofluidics.

In alternative embodiments, in practicing methods and systems of the invention, microencapsulated emulsions or droplets can be made using traditional methods, or by using an emulsifier (see for example: Griffiths, A. D. & Tawfik, D. S. Miniaturising the laboratory in emulsion droplets. Trends Biotechnol. 24, 395-402 (2006)). In alternative embodiments, methods and systems of the invention comprise use of droplet based microfluidics including high throughput droplet generators or multi-channel devices such as the TELOS SYSTEM™ from Dolomite Microfluidics (Royston, Herts, UK). In alternative embodiments, liquid droplets containing, for example, agarose or PEG, can be gelled or solidified to form droplet particles (see for example: Anal Chem. 2012 Jan. 3; 84(1):350-355). In alternative embodiments, in practicing methods and systems of the invention, highly parallel single-molecule amplification approach based on agarose droplet polymerase chain reaction can also be used for efficient and cost-effective aptamer selection, see, e.g.,).

### Droplet Based Screening

In alternative embodiments, the invention provides a drug screening and in vitro selection platform based on one type of molecule/one droplet strategy (see, e.g., FIGS. 39, 40, 41, 42, 43, 44, 45 and 46). We synthesized DNA, RNA and peptide in droplet library containing approximately 2×10¹¹ different sequences in diversity. We encapsulated in picoliter droplets (20 μm in diameter) synthesized DNA on microbeads; the on-bead DNA was amplified by PCR to generated a droplet DNA library. These DNA can then be transcribed and translated within the droplets to form RNA and peptide libraries. In particular, the identity/sequence of translated proteins/peptides can be barcoded in the same droplet using the nucleic acid sequences, which provides a powerful tool for subsequent screening. These facile, inexpensive exemplary libraries generated by methods and systems of the invention are valuable to screen and/or to obtain active biologics, such as therapeutics or diagnostics, and for biomarker discovery purposes.

### DNAzyme Sensors

In alternative embodiments, DNAzymes, also called “DNA enzymes” or “deoxyribozymes”, are used to practice the methods and systems of the invention. They are synthetic single-stranded (ss) DNA oligonucleotides with catalytic activities.^{11,12} In alternative embodiments, catalytic DNA molecules used to practice the invention can be generated in vitro from a vast random library using a combinatorial approach called in vitro evolution or selection^{13,14} where the properties of the molecules to be selected can be tailored and pre-defined.

In alternative embodiments, DNAzymes used to practice the methods and systems of the invention have diverse catalytic activities, including DNA/RNA cleavage, phosphorylation, and RNA ligation.¹² DNAzymes used to practice this invention can be made using any known technique, e.g., as described in U.S. Pat. Nos. 8,329,394; 8,450,103.

In alternative embodiments, DNAzymes used to practice the methods and systems of the invention is a RNA-cleaving DNA motif that can cleave a DNA-RNA chimeric substrate at a single ribonucleotide junction (see e.g.,: Fluorogenic DNAzyme probes as bacterial indicators. Ali M M, Aguirre S D, Lazim H, Li Y. Angew Chem Int Ed Engl. 2011, Apr. 11; 50(16):3751-4.).^{10,15} In alternative embodiments, this unique property allows use of DNAzymes as a platform for real-time fluorescent sensors (see e.g., Catalytic nucleic acid probes as microbial indicators CA 2829275 A1, PCT/CA2012/000205).

Microencapsulation Droplet Systems Integrated with a 3D Particle Detector

In alternative embodiments, 3D particle detectors or counters are used to practice the methods and systems of the invention, see e.g., and as described in, e.g., Gratton, et al. U.S. Pat. No. 7,973,294 (2011); U.S. Pat. No. 7,528,834 (2009); J. P. Skinner, et al., Rev Sci Instrum 2013, 84; I. Altamore, et al., Meas Sci Technol 2013, 24. In alternative embodiments, the invention provides microencapsulation droplet systems integrated with a 3D particle detector as illustrated, e.g., in FIGS. 1, 2, 14, 15, 17, 32, and 33.

A 3D particle counter used to practice this invention can detect fluorescent particles from mL volumes at single-particle sensitivity within minutes. Briefly, as shown in FIG. 17, the exemplary apparatus comprises a small, portable microscope that has a horizontal geometry and a mechanical part that holds a cylindrical cuvette with a diameter of 1 cm. Two motors provide rotational (ranging from 10 to 1100 rpm) and vertical up-and-down motion (ranging from 1 to 15 mm/s) of the cuvette. The excitation light generated by diode lasers (e.g., 469 nm or 532 nm) are focused at the volume of observation that is typically positioned relatively close to the inside wall of the cuvette. The emission from the sample is collected by the same objective, transmitted through the set of dichroic filters, focused by a lens into a pinhole and then collimated by a second lens to the photomultiplier tubes (PMT). The photodetectors measure the fluorescence signal originated from the fluorescent particles in the observation volume and generate a temporal profile of the fluorescence. A pattern recognition filter extracts the spikes that have the correct shape from all other noisy signals with very high signal/noise rejection, which allows us to achieve exceptionally reliable and accurate detection. The simple and innovative design of this instrument allows a rapid scan of a relatively large volume (100 μL) in about 0.01 ms. The rotation of the tube in a spiral motion for about 100 seconds effectively explores about 1 mL of the tube. In addition, given the large measured volume and that only the fast signals are detected, the fluctuations resulted from particle diffusion can be neglected. We also emphasize that using this optical setup we penetrate only 150 μm into the sample. Therefore, strongly scattering samples such as whole blood (even before dilution by sensor solution) that have a transmittance at 500 nm of about 10% for a 250 μm path length can be easily handled.

This system can robustly detect few particles/mL using fluorescent microbeads or Sytox orange-stained E. coli (see, e.g., Skinner, et al., Rev Sci Instrum 2013, 84; I. Altamore, et al., Meas Sci Technol 2013, 24)

In alternative embodiments, the invention provides methods and systems comprising a microencapsulation droplet system integrated with a 3D particle detector, e.g., as illustrated in FIGS. 1 to 33.

In alternative embodiments, the methods and systems of the invention comprise the following unique features, including some that cannot be easily achieved by traditional detection assays:
1) Low abundance markers (e.g., 1-1 million/mL);
2) Able to interrogate large sample volume (μLs to mLs) and high throughput;
3) Rapid (minutes to hours);
4) Broad detection range;
5) Multiplexable;
6) No or minimal sample preparation is required; blood or other biological samples can be directly encapsulated and analyzed without any enrichment or purification steps. In alternative embodiments, the assay can be performed in a single step, homogenous manner; this can ensure that all targets can be analyzed.

In alternative embodiments, methods and systems of the invention can analyze a biological sample which can comprise a biopsy sample, or a blood, serum, saliva, tear, stool, urine or CSF sample from an individual or a patient. In alternative embodiments, methods and systems of the invention can analyze any samples obtained from a food, water, soil, or an air source.

In alternative embodiments, in practicing methods and systems of the invention, the samples can be directly assayed with no or minimal (e.g., dilution) processing. Standard, established biological sample preparation processes including dilution, purification, enrichment, extraction, centrifugation, magnetic bead assays, and washing steps, although not required, can be integrated into assays, methods and systems of the invention.

In alternative embodiments, assays, methods and systems of the invention, can detect and analyze any target, including e.g., but not limited to: cells (e.g., cancer cell, stem/progenitor cell, immune cell), pathogens (e.g., bacteria, multi-drug resistant organisms (MDRO), tuberculosis (TB)), parasites, fungi, viruses (e.g., HIV), cell-derived vesicles (e.g., exosome, microvesicles, apoptotic bodies), nucleic acids (e.g., SNPs, mutations, expression), proteins (e.g., PSA), enzymes (e.g., MMPs), peptides, lipids, carbohydrates, polysaccharides, small molecules or metal ions.

In alternative embodiments, forms of target species detected by assays, methods and systems of the invention include e.g., cell surface (e.g., EpCAM, N-cadherin, CD44, CD24), intracellular, and secreted markers (cell secretome), cell free circulating markers (e.g., miRNA, DNA, protein markers), metabolic markers, mechanical markers (e.g. cell deformability, stiffness, cytoskeleton, etc).

In alternative embodiments, methods and systems of the invention can be used to detect or monitor a biological event, e.g. DNA hybridization, protein receptor-ligand interaction, enzyme-substrate interaction, and cell surface receptor dimerization (including both homo and hetero-clustering), co-localization, or interaction with soluble ligands and drugs and another cells.

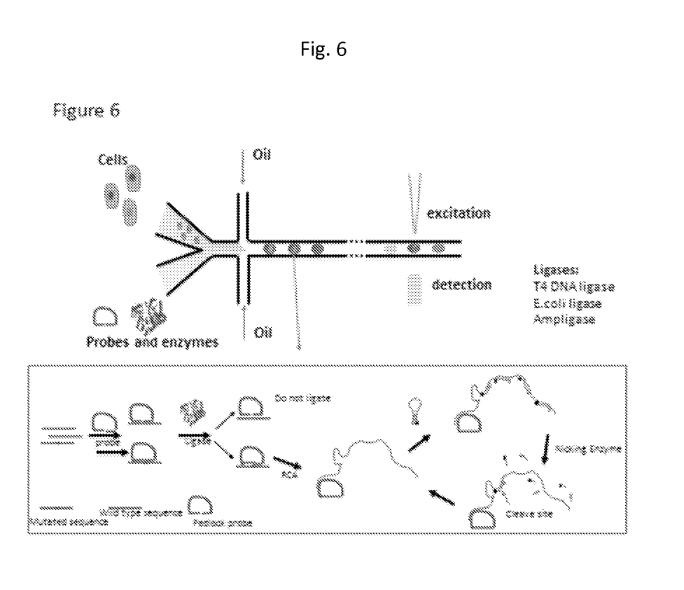

In alternative embodiments, methods and systems of the invention comprise use of a variety of detection assays for analyzing or measuring a target in a droplet. For example, methods and systems of the invention comprise use of a wide variety of established fluorescence bioassays, to e.g., selectively detect the targets within droplets for, e.g., the exemplary 3D particle counter analysis embodiments. Such assays include, both not limited to: nucleic acid based assays, antibody based assays, enzyme based assays, or chemical based assays or assays used in combination; or, nucleic acid based assays, including e.g., hybridization, molecular beacons, aptamer, DNAzyme, or other real-time fluorescent sensors; or, antibody-based assays, including, e.g., ELISA, sandwich based, immunostaining, antibody capture, secondary antibody amplification, or proximity ligation based; including e.g., enzyme based assays, including, e.g., PCR, RT-PCR, RCA, loop-mediated isothermal amplification (LAMP), nicking (e.g., EXPonential Amplification Reaction (EXPAR)), strand displacement, and exponential isothermal amplification (e.g., see Lab Chip, 2012, 12, 2469-2486) (a few examples are illustrated in FIGS. 6, 7, and 9). In some cases, the target itself such as PSA, MMPs, beta-lactamases, and carbapenemases can serve as enzyme to trigger a detection process (see FIG. 26 as an example).

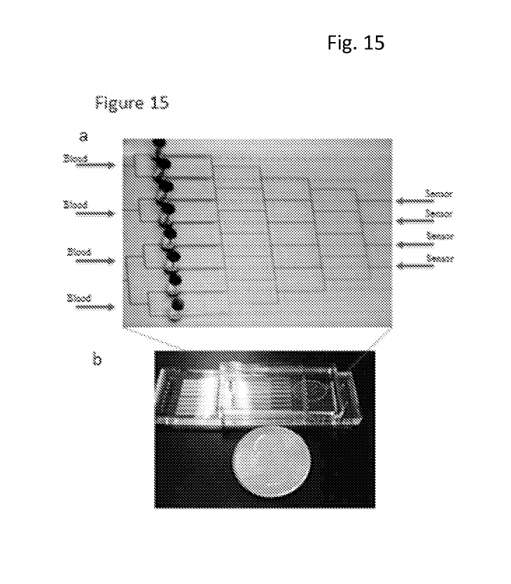

In alternative embodiments, in practicing methods and systems of the invention, microencapsulated emulsions or droplets can be made using traditional methods, or by using an emulsifier or by droplet based microfluidics. In alternative embodiments, methods and systems of the invention comprise use of droplet based microfluidics including high throughput droplet generators or multi-channel devices (see FIG. 15 for an example). Droplets can include water-in-oil formulations or the droplets can comprise water-in-oil-in-water (W/O/W) double emulsion formulations. In alternative embodiments, liquid droplets containing, for example, agarose or PEG, can be gelled or solidified to form droplet particles.

In alternative embodiments droplets are made in different sizes ranging from 10 nm to 100 s microns. Droplets can be manipulated in numerous ways including heating/cooling (for PCR), merging, splitting, sorting and long-term storage. Droplets can be analyzed by conventional 1D on-chip or 2D analysis, or by, in this invention, a 3D particle counter.

In alternative embodiments, in practicing methods and systems of the invention, any 3D particle counter can be used, e.g., comprising an instrument system as shown e.g., in FIG. 17 (labeled “3D particle counting system”), or a portable system for point-of-care applications (see, e.g., FIGS. 32 and 33).

In alternative embodiments, the invention provides integrated systems, e.g., systems engineered to comprise one or any of: desirable portability (for example, packaged as backpacks), automating fluid handing (i.e., droplet generation and auto sampling), and integrating electronics including a diode laser (light source), APD (detector), Operating (vinci, ISS Inc.) and/or data analyzing software (SimFCS), display, computer interface, smart phone, with a 3D particle counting system; e.g., as illustrated in FIGS. 32 and 33, which illustrate exemplary portable designs or embodiments of the invention comprising use of an integrated micro-encapsulator and 3D particle counting system.

In alternative embodiments, this exemplary device is integrated with multiple disposable microfluidic “cartridges,” permitting multiplex and rapid detection of multiple types of targets simultaneously. The device can be fully automated, and can be fabricated as an all-in-one system or with modular components. It can also be linked to smart phone and bluetooth etc for point-of-care applications, as illustrated in FIG. 32.

In alternative embodiments, to enable multiplex and parallel detection of multiple targets, our device can be comprised of multiple laser sources and detectors capable of reading at different wavelengths. The multiplex system permits simultaneous reading of multiple sensors (labeled in different colors) that are coded for different targets. In alternative embodiments, a carousel can be integrated in our apparatus to accommodate multiple sample vials for carrying out parallel tests.

Applications of Microencapsulation Droplet Systems Integrated with 3D Particle Detectors, or Integrated Comprehensive Droplet Digital Detection (IC 3D) Systems of the Invention

In alternative embodiments, the exemplary systems of the invention comprising an integrating droplet system and a 3D particle counter system, including the so-called “Integrated Comprehensive Droplet Digital Detection (IC 3D) system of the invention” (see e.g., FIGS. 1 and 33) permits selective detection of target species in biological samples in mL volume within minutes. In alternative embodiments, the exemplary systems of the invention revolutionize how we detect and analyze low concentration biological particles and markers. In alternative embodiments, the exemplary systems of the invention are utilized in a large variety of detection bioanalysis and diagnosis applications including, but not limited to:

Infectious disease pathogens (e.g., bacteria, viruses, fungi, etc), including skin infection, wound infections, diabetic ulcer infections, HIV, bacteria, TB, MDROs (e.g. MRSA);

Cancer;

Diabetes;

Alzheimer disease (e.g., Amyloid beta, Tau proteins);

Brain injury and disorders (e.g., S100B, a glial-specific protein, where elevated S100B levels accurately reflect the presence of neuropathological conditions including traumatic head injury or neurodegenerative diseases)

Inflammatory and autoimmune diseases (e.g., CD4 T cell, immune cell count);

Stem cell and regenerative medicine (e.g., mesenchymal stromal cells, endothelial progenitor cells, hematopoietic stem cell, cells can include both endogenous and exogenously transplanted cells);

Cardiovascular diseases (e.g., C-Reactive Protein (CRP), B-type natriuretic peptide (BNP), troponin, Cystatin C, IL-6);

Drug and abuse (e.g. Tetrahydrocannabinol, THC);

Newborn screening (e.g., phenylalanine).

In alternative embodiments, the exemplary systems of the invention are used to study new biology, cell-drug interactions and drug susceptibility, to develop new drugs and therapeutics and monitor disease progress and treatment efficacy or used as companion diagnostics and to be used in sequencing, personalized diagnostics and medicine. In addition to medical applications, exemplary systems of the invention can also be used for other areas including food industry, agriculture, water systems, air systems, and defense applications.

### Rapid and Sensitive Bacteria and Antimicrobial Resistance Detection Methods to Expedite Blood Infection, e.g., BSI, Diagnosis and Treatment:

The invention provides systems and methods for the rapid and sensitive identification of bacteria in blood, which will significantly reduce the mortality rate and the cost of medical care associated with blood infections.

In alternative embodiments, the invention provides rapid and sensitive methods to detect blood stream infections in order to expedite blood infection diagnosis and treatment.

In alternative embodiments, the invention provides rapid and sensitive methods to detect antimicrobial resistances including extended spectrum beta-lactamase (ESBL) and carbapenem-resistant Enterobacteriaceae (CREs).

### Cancer Detection and Monitoring:

In alternative embodiments, the invention provides rapid and sensitive methods to detect cancer cells, e.g., to detect a metastasis, or a dissemination of cancer cells from the primary tumor to other organ sites, e.g., to detect the formation and growth of a primary tumor, e.g., to detect cancer cells that are shed from the primary tumor into the circulation known as circulating tumor cells (CTCs). In alternative embodiments, the invention provides methods for the analysis and quantification of CTCs for early-stage diagnosis, prognosis and monitoring disease course. In alternative embodiments, the invention provides methods for detecting cancer markers such as proteins (e.g., Prostate-Specific Antigen (PSA)), cell-free nucleic acids (e.g., DNA, mRNA, miRNA), cell derived particles (e.g., exosomes, microvesicles, apoptotic bodies). In alternative embodiments, the invention provides methods for detecting very rare markers, for example, where one CTC is present per 10⁷ leukocytes. Methods of the invention can be used with or in place of heterogeneous, traditional flow cytometry, DNA and RNA sequencing, and immunological approaches (e.g., a CELLSEARCH™ platform) to, e.g., reliably detect cancer markers such as CTC or PSA in clinical settings.

In alternative embodiments, the invention provides single-cell detection methods that can offer a way to dissect the heterogeneity of cancer cells. In alternative embodiments, the invention provides the ability to detect and analyze rare cells at a single cell level, including detection of nucleic acids, proteins, and metabolites for personalized diagnostics and treatment.

### Detection and Monitor Brain, Neurological and CNS Diseases and Disorders

In alternative embodiments, the invention provides methods for detecting established biomarkers for neurological and central nervous system (CNS) diseases and brain tumors, trauma and injury. In alternative embodiments, the invention provides methods for detecting the accumulation of amyloid-β (Aβ) peptides (i.e., Aβ42 and tau proteins, which are two key neuropathological features characterizing the Alzheimer's disease (AD) brain and may be important biomarkers that are detected in CSF characterizing AD pathogenesis. In alternative embodiments, the invention provides methods to detect and quantitate these biomarkers, which can be invaluable to studies that aim to use Aβ and tau proteins as biomarkers to 1) screen and monitor AD, 2) better understand the molecular biology and pathology of the disease, and 3) evaluate therapeutic interventions. In alternative embodiments, the methods of the invention can be used in place of or with existing assays including ELISA to e.g., detect Aβ and tau protein. In alternative embodiments, the invention provides screening and detection of such markers in blood and urine, including any marker such as S100B (S100 calcium binding protein B) that is at a very low concentration, and often cannot be detected by existing assays because of the blood brain barrier (BBB).

### Residue HIV Detection

In alternative embodiments, the invention provides methods for detecting and characterizing retroviruses, e.g., human immunodeficiency virus (HIV), HIV/antibody complexes and rare reservoir cells containing HIV. Recently, there were a few incidents where HIV patients seemed to be cured by new treatments including bone marrow transplantation. However, HIV returned after several months. A major challenge is that during therapy the viral particle concentration can often go below the detection limit of existing technologies, which appears to be “cured” but actually not. Therefore, methods of this invention can detect extremely low numbers of viral particles to aid in this therapy and prognoses.

### Droplet Microencapsulation Systems

In alternative embodiments, the invention provides methods and systems comprising use of droplet emulsion encapsulation (e.g., water-in-oil), which is an established method to compartmentalize samples and agents in small volumes for a variety of purposes including bioassays, drugs and food industry. In alternative embodiments, the invention provides methods comprising use of multiphase flows in microfluidic systems as a platform for ultra-sensitive and high-throughput screening and experimentation.

In alternative embodiments, methods of the invention use “droplet microfluidics” to enable the generation and manipulation of monodisperse, microdroplet, e.g., picoliter-sized, liquid droplets in an immiscible carrier oil fluid (e.g., water-in-oil emulsion) (see e.g., “Droplet microfluidics for single-molecule and single-cell analysis for cancer research, diagnosis and therapy”, Dong-Ku Kang et al. Trends in Analytical Chemistry, 2014). In alternative embodiments, methods of the invention utilize compartmentalization in picoliter droplets (e.g., 1 to 300 μm in diameter) to increase assay sensitivity and decrease assay time by increasing the effective concentration of target species.

In alternative embodiments, droplet microfluidics is used for high-throughput and multiplex detection and analysis of low concentrations of targets such as single cells; and detection of gene expression, cell viability and proliferation, cell-cell and cell-drug interactions at a single-cell level. In alternative embodiments, droplets are manipulated in numerous ways, including heating/cooling (for PCR), merging, splitting, sorting and long-term storage.

In alternative embodiments, methods of the invention comprise multiple (for example, up to 256) droplet generating channels which is able to convert 1 mL sample into droplets within several min.

In alternative embodiments, methods of the invention comprise encapsulation of gelable materials, such as agarose, which can be easily fabricated to form hydrogel droplets for different purposes including repetitive washing and reaction steps and flow cytometry analysis; droplets can be detected on-chip and efficiently sorted with high-throughput, for example, at greater than 1000 droplets/second (s).

### 3D Particle Detector

In alternative embodiments, methods of the invention comprise use of a 3D particle detector, also called a Rare Event Detector, a 3D particle scanner or a fluorescence correlation spectroscopy (FCS), e.g., as described in U.S. Pat. No. 7,528,384; U.S. Patent application publication no 20090230324; U.S. Pat. No. 7,528,384. In alternative embodiments, such 3D Particle Detectors are able to achieve a throughput that is clinically relevant. In alternative embodiments, methods of the invention comprise use of 3D particle counting techniques that can detect particles (e.g., fluorescent beads or dye-stained cells) from milliliter (mL) volume at single-particle sensitivity within minutes.

In alternative embodiments, methods of the invention comprise use of 3D particle counting techniques that can rapidly scan of one mL of fluid by moving a tube containing the fluid in a spiral motion in front of an objective of the confocal microscope. The optics of the microscope can be designed to measure a relatively large volume (100 pL) in about 0.01 ms. The rotation of the tube in a spiral motion for about 100 seconds effectively explores about 1 ml of the tube. The rapid passage of the fluorescent particle in the volume of excitation produces a very strong signal with signal-to-noise ratio (S/N) greater than 100. Since only the fast signals are detected, the slow modulation of the fluorescence signal due to the imperfections in the mechanical construction of the rotating tube has no effect on the S/N, this system can robustly detect few particles/mL using fluorescent microbeads or Sytox orange-stained E. coli, e.g., as described in Skinner, Rev Sci Instrum., 84(7), 074301; Altamore (2013) FCS. Meas Sci Technol., 24(6), 65702.

The invention will be further described with reference to the following examples; however, it is to be understood that the invention is not limited to such examples.

### EXAMPLES

### Example 1

### Detection of Bacteria in Biological Samples Using Microencapsulated Sensors

Real-time fluorescent DNAzyme sensors: In one embodiment, a DNA library containing approximately 10¹⁴ random sequences (e.g., chemically synthesized) are used 5 for selecting and/or isolating DNAzyme sensors. The library can consist of a variable sequence, e.g., of about 40 nucleotides that is ligated to a fluorogenic, DNA-RNA chimeric substrate (see FIG. 10a).⁷ The substrate can contain a single ribonucleotide (riboadenosine) as a cleavage site that is flanked by a fluorophore and a quencher on each side. The rationale is that specific DNA sequences in the library (i.e., DNAzymes) exist and are cleaved at the ribonucleotide linkage, therefore producing a fluorescence signal only in the presence of target bacteria lysates.

In vitro selection can be by incubating a starting library with a target bacterial lysate for about 10 min in HEPES buffer. The cleaved molecules can be gel isolated, amplified by primer-specific PCR, ligated to the substrate and then used in the next round of selection. Bacterial lysates from non-target bacteria can be included as a negative selection to remove nonspecific DNAzymes and ensure assay specificity. In our experience, 8 to 15 rounds of selection (approximately 1-3 months) are needed for the completion of a selection.⁷ The final round of the DNA pool can be sequenced. Using this selection approach, real-time DNAzyme sensors that rapidly detect a variety of bacteria including E. coli, Listeria, Salmonella and Clostridium difficile have been isolated. Such high selectivity demonstrates that by including appropriate negative selection targets in the selection process, it is feasible to generate DNAzyme sensors that specifically detect a particular bacterium, MDRO or other pathogens. In alternative embodiments, methods and systems of the invention incorporate any known method using fluorogenic DNAzyme probes as a cell, e.g., a bacterial, indicator, e.g., as described in Ali et al, Angew Chem Int Ed Engl. 2011 Apr. 11; 50(16):3751-4; or, Li et al., WO/2012/119231.

We used these rapid, fluorogenic DNAzyme sensors as an example in our system. As shown in FIG. 10a and b, the sensor contains a DNAzyme domain that is ligated with the DNA-RNA chimeric substrate where the ribonucleotide cleavage site is flanked by a fluorophore and a quencher. This “inactive” state has a minimal fluorescence signal due to the close proximity of the fluorophore and the quencher. In the presence of target bacteria, E. coli used herein as a model system, DNAzymes will bind to target molecules produced by bacteria and cleave the substrate. The cleavage event frees the fluorophore from its quencher, thereby generating a high fluorescence signal. Moreover, the DNAzyme sensor is able to distinguish target E. coli from control bacteria or mammalian cells with high selectivity (FIG. 10c). We further demonstrate that the DNAzyme sensors previously isolated using stock isolates of E. coli can robustly and selectively detect clinical E. coli isolates that were spiked and then lysed in blood (FIG. 10d). It is interesting to note that although the DNAzyme sensor can detect all clinical E. coli samples, the fluorescence intensity varies between samples, which might reflect the potential molecular heterogeneity between different E. coli strains. This also suggests that by including appropriate positive and negative selection targets in the in vitro evolution process, it is feasible to generate DNAzyme sensors that can distinguish different strains of the same bacterium species.

Since our goal is to develop a “mix-and-read” assay that uses whole blood with no or minimal sample processing, we further tested the sensor performance in whole blood and found that our Fluorescein/Dabcyl modified DNAzyme sensors produced a sufficiently high fluorescence signal in response to E. coli spiked in blood that was diluted by sensor solution to various volume ratios (FIG. 11a) with a 10% final blood concentration determined to be optimal and therefore used in subsequent droplet experiments (below). Optimization of dye pairs especially using near infrared dyes that are less interfered with by blood autofluorescence can further improve sensor performance (e.g., signal/noise ratio) in blood. We further demonstrated that the DNAzyme sensors exhibited sufficient stability in blood within the time frame (<1.5-4 hours) we target for future clinical use (FIG. 11b). In alternative embodiments, the termini or backbone of DNAzymes (i.e., inverted T and phosphorothioates) can be further chemically modified; or, RNase inhibitor (ribolock, Fermentas) can be included, in the assay buffer to further increase their blood stability.

Given that blood stream infections (BSIs), sepsis and antimicrobial resistance can be caused by several different types of pathogens, the sensor set can be expanded through in vitro DNAzyme sensor selection described above to detect the other pathogen species. In particular, the nonbiased screening using bacteria as a complex target without prior knowledge of any specific target molecules bypasses the tedious process of purifying and identifying target molecules from extremely complex mixtures and permits the rapid development of sensors for new bacterial strains in an unanticipated outbreak. This addresses a major challenge faced by existing techniques including PCR that rely on the detection of pre-identified target genes or other biomarkers given the rapid and complex evolving mechanisms associated with bacteria. Although the identification of specific bacteria biomarkers that bind to DNAzymes to trigger substrate cleavage is not necessary for our assay to operate, they can be identified in using affinity purification coupled with mass spectrometry.

In alternative embodiments the invention uses a panel of real-time, fluorogenic DNAzyme sensors, which can be make via in vitro selection using e.g., major blood-infection bacteria or drug resistant organisms as targets including e.g., Staphylococcus aureus (S. aureus), Enterococcus faecalis (E. faecalis), coagulase-negative Staphylococci, Klebsiella pneumoniae, Acinetobacter baumannii, Pseudomonas aeruginosa, Enterobacter species and extra-intestinal pathogenic Escherichia coli, ESBLs, CREs, methicillin-resistant Staphylococcus aureus (MRSA), and fungal pathogens.

Droplet microfluidics: In alternative embodiments, the systems and methods of the invention manipulate multiphase flows in microfluidic systems as a platform for ultra-sensitive and high-throughput screening and diagnostics. These systems, called “droplet microfluidics”, enable the generation and manipulation of monodisperse, picoliter-sized liquid droplets in an immiscible carrier oil fluid (i.e., water-in-oil emulsion).¹¹⁻¹⁴ The ability to controllably generate droplets with variable analyte composition, and at high rates, makes droplet microfluidics a powerful tool to address a range of chemical and biological applications including enzymatic assays, protein crystallization, nanomaterial synthesis, and cell-based assays.¹¹⁻¹⁴ The compartmentalization in picoliter droplets (which is alternative embodiment can be between about 1 to 300 μm, or 10 to 100 μm, in diameter) increases assay sensitivity and decreases assay time by increasing the effective concentration of target species.¹¹ Therefore, in alternative embodiments droplet microfluidics is particularly suited for high-throughput and multiplex detection and analysis of low concentrations of targets such as single cells. Indeed, gene expression, cell viability and proliferation, cell-cell and cell-drug interactions at a single-cell level have been demonstrated using droplet microfluidics.¹² In alternative embodiments, droplets are manipulated in numerous ways including heating/cooling (for PCR), merging, splitting, sorting and long-term storage. In alternative embodiments, droplets can be detected on-chip and efficiently sorted with high-throughput (>1000 droplets/s).¹¹

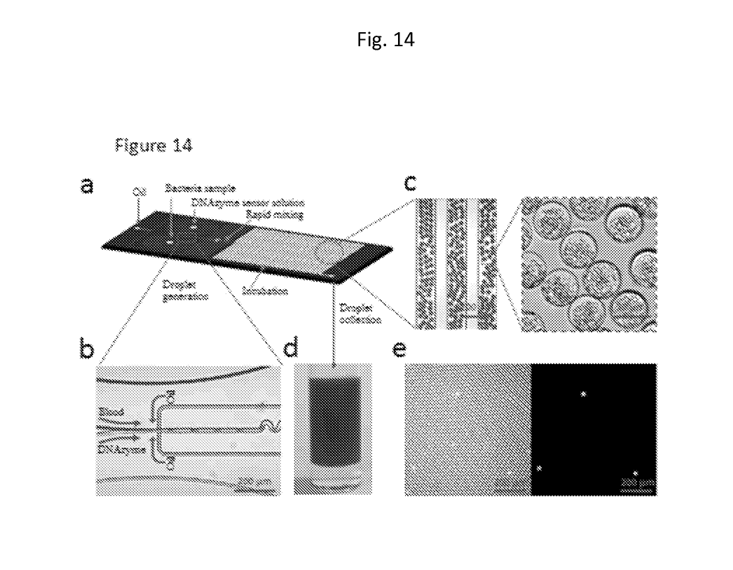

In alternative embodiments, the systems and methods of the invention can detect bacteria in patient blood at single-cell sensitivity within minutes, as illustrated in FIGS. 12, 13, 14 and 16. In alternative embodiments, the systems and methods of the invention integrate bacterium-detecting DNAzyme sensors, which are obtained by in vitro selection, with droplet microfluidics (FIG. 14). In alternative embodiments the confinement of bacteria in droplets significantly increases the concentration of released target molecules that can be detected by the DNAzyme sensors in a rapid, real-time fashion.

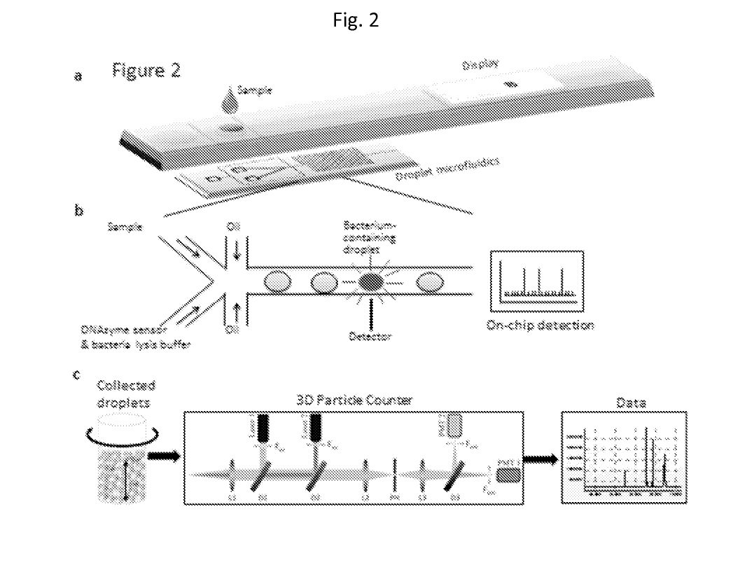

FIG. 2a illustrates an exemplary automated device of the invention for routine bacteria detection and screening. Patient blood sample is analyzed and the number of target bacteria in the sample are shown on the display panel within several minutes to hours. Droplet microfluidics is integrated with a DNAzyme sensor system for detecting bacteria in blood. Bacterium containing fluorescent droplets can be counted on-chip (FIG. 2b) or, after collected to a cuvette, by a 3D particle counter (FIG. 2c) (Example 2, below).

In alternative embodiments, blood sample and DNAzyme sensors are mixed and then encapsulated in hundreds of millions to billions of micron-sized droplets. DNAzyme sensors produce an instantaneous signal in the droplets that contain bacterium, which will be counted and analyzed. In alternative embodiments, patient blood is mixed with DNAzyme sensor solution, including bacteria lysis buffer, within the microfluidic channel, which can be encapsulated in millions of individual picoliter droplets (FIG. 2b). Because bacteria exist at low numbers in blood (typically 1-100 CFU/mL), each droplet may contain one or no bacteria. DNAzyme sensors can fluoresce instantaneously in the droplets that contain bacterium. The droplets can be detected by an embedded APD (photon avalanche diode) in a high throughput manner (approximately 3000 droplet count/s). The system can also be integrated with multiple droplet microfluidics “cartridges” which will permit screening for multiple major bacteria targets simultaneously.

In alternative embodiments, the in vitro selection technique can generate multiple DNAzyme sensors for various major pathogenic bacteria, making multiplex bacterial detection possible. The compartmentalization of a single bacterium in a droplet significantly increases the concentration of target molecules, permitting rapid detection and single-cell sensitivity. The significantly shortened assay time (i.e., minutes instead of hours to days in the conventional techniques) allows blood infections to be treated timely and effectively.

In alternative embodiments, an exemplary platform of the invention can also be easily integrated with drug susceptibility screening assays to identify the best antibiotics regimen for patient-specific treatment. Such rapid detection and early intervention can significantly improve the chances of treating blood infections and reduce mortality. Thus, the invention can significantly increase the survival of patients with blood infections and decrease the financial costs associated with patient care.

In alternative embodiments the rapid and single-cell detection methods and systems of the invention can serve as a platform for the detection and screening for slowly-growing species (e.g., Mycobacterium tuberculosis) and other rare cells in blood such as circulating tumor cells.

### Droplet Microfluidics Fabrication and Setup

Device fabrication: Droplet microfluidics can be fabricated and operated following any known and established procedures, e.g., as discussed above.²⁶ For example, in one embodiment, a poly(dimethylsiloxane) (PDMS) chip with 20 μm-depth and 15 μm-width channels is fabricated using standard soft lithography, and mounted on a glass microscope slide. As illustrated in FIG. 14a, the PDMS device has one oil inlet and two aqueous inlets (one for bacteria spiked buffer or blood with the other one for DNAzyme sensor and cell lysis reagents). Standard pressure infuse/withdraw syringe pumps is used to deliver reagents and oil at flow rates ranging from 0.5 to 2 μL/min. Uniform picoliter-sized droplets are generated at a rate of approximately 50 Hz by flow focusing of the resulting stream with HFE-7500 fluorinated oil containing 2% (w/w) EA surfactant. Droplets with three different sizes (10, 20 and 50 μm in diameter) can be generated, which in alternative embodiments the different sizes are made by tuning the microfluidic channel size and flow rate. FIG. 14c shows a representative image that demonstrates 30 μm droplets are being generated. Following droplet formation, a short “wiggle” module is incorporated for rapid mixing of droplets by chaotic advection (FIG. 14a). The droplets will then flow through the “incubation channel” (70 cm) before they are detected at the dewtection zone.

FIG. 14 illustrates: (a) an exemplary layout of a droplet microfluidic chip; (b) an exemplary/representative microscopy image showing uniform microdroplets being formed; c), blood contents especially red blood cells are clearly visible in droplets. d) Droplets collected in the cuvette. e) Representative fluorescence microscope images demonstrate DNAzyme sensors (250 nM) light up the droplets that contain single E. coli K12 in 10% blood after 3-hour reaction.

In alternative embodiments, the droplets in our system can be made via high throughput droplet generator with multiple droplet generation challenges or structures. In alternative embodiments, the high throughput droplet generator permits conversion of a milliliter sample into droplets within several minutes. As illustrated in FIG. 15: illustrating an example of a high-throughput blood micro-encapsulation device: double layer microfluidic device was designed to integrate 8 droplet generators within single device; microfluidic devices were fabricated using Polydimethylsiloxane (PDMS) by soft-lithography method; sensor and Blood samples were introduced from the top layer and oil was injected from bottom layer. Sensor and blood were merged at the middle of the top layer and they were down through the interconnecting hole to the bottom layer. Mixed samples were formed droplets from flow-focusing structure on the bottom layer by given oil and generated blood droplets were collected for droplet counting.

In alternative embodiments, the use of larger droplet and smaller blood dilution factor can further significantly reduce the droplet generation time.

In alternative embodiments, droplets can be gelable materials, such as agarose, which can be easily fabricated to form hydrogel particles for different purposes including repetitive washing and reaction steps and flow cytometry analysis.

Droplet detection and quantification: Fluorescence measurement of droplets can be carried out by using a custom-built confocal microscope (Observer Z1™, Zeiss). This confocal setup consists of 488 and 561 nm diode lasers as excitation sources, and an electron multiplying charge coupled device (QuantEM:512SC, Photometics) for fluorescence detection. In order to maximize the scanning speed, a CSU-XI spinning disk (CSU-X1, Yokogawa, Japan) is integrated into the confocal microscope. Typically, droplets will be measured at a rate of 100 s to 1000 s droplets per second and the data can be analyzed using ImageJ. In addition to confocal microscopy, standard flow cytometry can also be used to analyze, quantify and sort fluorescent droplets in a high throughput manner.³⁵

High Throughput Droplet Detection:

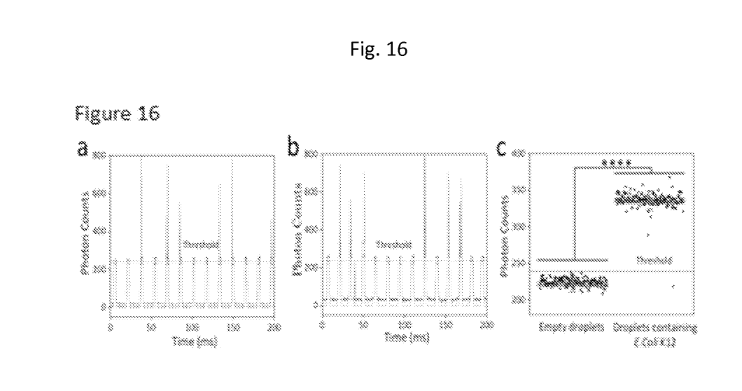

To achieve high throughput detection, In alternative embodiments, an optical system which incorporates a highly sensitive APD detector with a dual-band emission filter (z488/635, Chroma Technology Corporation, USA) and dichroic mirror (630dcxr, Chroma Technology Corporation, USA) is used; this can count droplets at a throughput of ˜3000 droplets/s (see FIG. 16). In alternative embodiments the optical system can consist of mirrors that reflect and transmit the light source and fluorescence emission from the sample prior to detection. Before reaching the detectors, the fluorescence emission will pass through the dual-band emission filter, removing residual excitation light, and the dichroic mirror will split the fluorescence emission into two paths to be simultaneously detected by the APD detector.³⁸ The optical system can be incorporated into the confocal microscopy system for high throughput droplet analyses.

### Optimizing Bacteria Detection in Buffer

Detection of bacteria in droplets using DNAzyme sensors: Droplet microfluidic systems integrated with DNAzyme sensors can be optimized to detect bacteria in reaction buffer, using e.g., 50 mM HEPES, pH 7.5, 150 mM NaCl, 15 mM MgCl₂, and 0.01% Tween 20. Two important properties can be targeted: sensitivity and detection time. As bacteria exist at low numbers in patient's blood (typically 1-100 CFU/ml), when encapsulated in picoliter droplets, each droplet will contain one or no bacterium. Therefore, significant that the systems of this invention can detect bacteria at a single-cell sensitivity. In alternative embodiments, target bacteria such as E. coli, are encapsulated together with their respective DNAzyme sensors (e.g., at 100 nM, modified with Fluorescein and Dabcyl) into droplets. Control experiments including mutant DNAzyme/target bacteria and DNAzyme sensor/non-target bacteria can be included to assess the specificity of a droplet assay. Lysozyme (1 mg/mL), a bacteria lysis agent, can be pre-mixed in the DNAzyme sensor solution. The use of lysis agents allows the target molecules to be rapidly released from bacteria, which will further decrease the assay time. Bacteria lysis conditions can be systemically optimized using various agents including e.g., Triton X-100, IGEPAL, SDS and lysozyme alone or in combination, and identified that lysozyme most efficiently lyses bacteria without interfering with droplet formation or DNAzyme sensor function.

Bacteria can be statistically diluted and compartmentalized in droplets at a range of concentrations. For example, for a 50 μm droplet, the initial cell concentration will be 3, 30, and 300×10⁶/mL bacteria in order to form 1, 10, and 100 bacteria per droplet. When the initial bacteria solution is extremely diluted (<3×10⁶/mL), the formed droplets will contain single or no bacteria per droplet. Bacteria can be stained with Syto9 (green) or Syto17 (red), which allows better visualization of them within droplets to quantify the numbers of cells per droplet using confocal microscopy. Staining bacteria with a different color allows co-localization with the DNAzyme sensor signal in the same droplet in the detection assay.

Bacterium-containing droplets can be easily detected due to the intense fluorescence signal produced by DNAzyme sensors. We have shown that an exemplary E. coli sensor of the invention can detect bacteria in droplets, with the signal directly correlated to the number of cells per droplet (FIG. 12d). We first demonstrate that, in buffer, the DNAzyme sensor system is able to detect single target E. coli K12 that is lysed in a droplet (5 μm in diameter) within 8 min with a comfortably high signal/background ratio of ˜4 (FIG. 12a-c). We attribute this single-cell sensitivity and reduced detection time in droplets compared to those of bulk assays to the increased target concentration via single-cell confinement. Single-cell detection can be optimized for any target bacteria using their respective sensors through both confocal microscopy and high-throughput flow cytometry techniques.

Optimization: Optimal detection time and signal/background ratio of the droplet assay for a particular assay or target can be achieved by optimizing two parameters: droplet volume (or size) and DNAzyme sensor concentration. As smaller droplet sizes lead to higher target concentrations from single cells, increasing the signal/background ratio and decreasing the detection time, the performance of three different droplet sizes of e.g., 10, 20 and 50 μm can be specifically compared. For the droplet assay, a DNAzyme sensor concentration of 100 nM can be a starting point, which has been shown to be optimal in a bulk assay. DNAzyme sensor concentrations, e.g., at 10, 50, 100, 200 and 500 nM, can be optimized to reach the best balance of detection time and signal/background ratio.

Examine and optimize bacteria detection in spiked blood: In alternative embodiments, exemplary systems of the invention are used to detect bacteria in unprocessed (or diluted) blood. DNAzyme sensors can be modified with dye-quencher pairs that are compatible with blood detection. For titrating and optimization, bacteria can be spiked in undiluted whole blood in various concentrations, which will be encapsulated along with DNAzyme sensor solution into droplets as described above. To prevent clotting and precipitation of blood sample during injection, a 2 mm magnetic bar can be placed inside the syringe with a portable magnetic stirrer placed on the top.

Whole blood containing bacteria can be directly encapsulated into droplets, as illustrated in FIG. 14c and d. This can be stably stored for days to months at room temperature. The volume ratio between blood and sensor solution in the droplet system can be optimized for any given assay. This can be easily achieved by tuning the flow rates between blood and DNAzyme sensor solution, to produce the optimal signal/background ratio without compromising the throughput (i.e., the amount of whole blood processed per time). For detection of bacteria in blood, we can optimize droplet size: while smaller droplet sizes lead to higher target concentrations from single cells (which would increase the signal/background ratio and decrease the detection time), it is technically challenging to encapsulate blood contents including red and white blood cells into too small sized droplets. We determined that droplets 25 μm in diameter are optimal for this purpose and therefore used for subsequent blood droplet experiments.

In alternative embodiments, the invention provides compositions and methods comprising use of droplet microfluidics with a DNAzyme sensor system to selectively detect single bacterium, e.g., in buffer and/or spiked blood. Using fluorescent microscopy (FIG. 14e) or 1D on-chip droplet counting system (FIG. 16), our system is able to selectively detect single target E. coli K12 in 10% blood in droplets. Furthermore, by colocalizing with the Syto17 signal, we observe that our encapsulated DNAzyme sensor system possesses zero false positive rate and minimal false negative rate (˜0.5%) from ˜70,000 droplet counts in triplicate experiments we performed using E. coli K12 as positive target and sensor alone or control bacteria as negative controls (FIG. 16). Finally, a measurable fluorescence signal can be observed <3 hours in response to a single bacterium in blood (FIG. 14e).

In case a single emulsion droplet (water-in-oil) is not compatible with a flow cytometry system, a water-in-oil-in-water double emulsion droplets can be used (fabricated) for that set of flow cytometry measurements. Water-in-oil-in-water double emulsion droplets can be easily fabricated using two flow-focus junction devices, and have been widely used for flow cytometry analysis and sorting. We did not observe blood clogging in the channel before encapsulation in the droplets. If necessary, that part of channel can be coated with non-fouling polyethylene glycol (PEG) or heparin to further minimize undesirable clogging of blood components.^{36,37}

Detecting bacteria from clinical specimens: In alternative embodiments, the invention provides compositions and methods having clinical applicability.

Using patient samples: In alternative embodiments, the invention provides compositions and methods, including devices, able to determine the presence of bacteria with very high sensitivity and specificity. By determining the type and/or presence of bacteria, appropriate antibiotic treatment can be determined—and monitored during the course of therapy. An aliquot from a blood culture is transferred to a sterile 15 ml conical tube; patient blood (e.g., about 1 mL) that may contain or contains a particular type of bacteria can be encapsulated into droplets with its respective DNAzyme sensors, e.g., following the optimized protocols, as discussed above. The fluorescent droplets can be counted by the high throughput APD detector. We can analyze a total of e.g., 10 patient samples for each bacteria target. A set of experiments can be performed to allow determination of whether any particular system can reliably detect bacteria in patient blood samples, e.g., the false positive and negative rate.

Thus, in alternative embodiments, methods, systems and devices of the invention can reliably detect bacteria from patient samples with high sensitivity and selectivity (<10% false positive and false negative rates).

Portable system: In alternative embodiments device are portable and provide automating fluid handing (i.e., droplet generation), and integrating electronics including a light source (thin film LED), diode detector, and detector display (FIG. 2a).^{38,39} This exemplary device can be integrated with multiple disposable microfluidic “cartridges,” permitting multiplex and rapid detection of multiple types of bacteria involved in blood infections simultaneously.

In alternative embodiments, methods, systems and devices of the invention can detect multidrug-resistant organism (MDRO) or antimicrobial resistant infections, which are a major global health problem and pose a particular challenge to the care of combat- and trauma-wounded personnel.¹⁻² In alternative embodiments, methods, systems and devices of the invention provide early identification of MDROs, which is crucial for improving patient care by preventing the spread of disease and identifying appropriate antibiotic treatment.³ In alternative embodiments, methods, systems and devices of the invention can be used in place of, or to supplement, bacterial cultures (which require days to get a result) and/or amplification-based molecular diagnosis methods such as polymerase chain reaction (PCR; which can reduce the assay time to hours but are still not sensitive enough to detect bacteria that often occur at low concentrations, e.g., 1-100 colony-forming unit (CFU)/mL in infected blood.^{4,5} In alternative embodiments, methods, systems and devices of the invention can be used routine screening of MDROs, or under resource-limited environments, such as in third world countries, emergencies, disaster situations or battlefields.

### Example 2

### Detecting Bacterial Infections Using 3D Particle Counter-Integrated Systems i.e. the IC 3D

The following describes an exemplary method of the invention for detecting a blood stream infection (BSI), and rapidly detect, identify and thus treat bacteria in the early stages of infection.

We have demonstrated that the integrated droplet system and 3D particle counter system of this invention allows selective detection of bacteria in unprocessed or minimally processed buffer and blood samples at single-cell sensitivity within minutes to several hours. In this example, our system integrates DNAzyme sensor technology, droplet microfluidics and a high throughput 3D particle counting system (i.e., Integrated Comprehensive Droplet Digital Detection (IC 3D)) (FIGS. 1 and 2c). This exemplary combination permits selective detection of single cells in blood in mL volume within minutes.

In alternative embodiments, patient whole blood or other biological samples such as urine are mixed with a DNAzyme sensor solution, including bacteria lysis buffer, within the microfluidic channel, which will be encapsulated in hundreds of millions to billions of individual picoliter droplets, as illustrated e.g., in FIG. 2b.

DNAzyme sensors are short catalytic oligonucleotides that are identified by in vitro selection to specificity react with the lysates of target bacteria, leading to a rapid, real-time fluorescence signal. In alternative embodiments, E. coli-specific DNAzyme sensors are used in this example to selectively detect E. coli (FIG. 10). In alternative embodiments, exemplary in vitro selection techniques can generate multiple DNAzyme sensors for various major pathogenic bacteria, making multiplex bacterial detection possible. Specifically, patient blood will be mixed with the DNAzyme sensor solution including bacteria lysis buffer within a microfluidic channel, which will be encapsulated in millions to billions of individual picoliter droplets. DNAzyme sensors will fluoresce instantaneously in the droplets that contain bacterium, which will be detected and counted by a high throughput 3D particle counting system that can robustly and accurately detect single particles from mL volume within several minutes (FIG. 2b and c). The resultant fluorescent droplets can then be detected with exceptionally high reliability and clinically relevant throughput.

In alternative embodiments, the compartmentalization of a single bacterium in a droplet significantly increases the concentration of target molecules, permitting rapid detection and single-cell sensitivity without signal amplification processes such as PCR. In alternative embodiments, such compartmentalized, target-specific reaction is a critically necessary step to “light up” the droplets that contain target bacteria so that they can be detected by the 3D particle counting system. In alternative embodiments, the exceptional reliability and accuracy of exemplary 3D particle counting systems of the invention for single droplet analysis in mL volume within minutes bypass many challenges faced by current particle counting techniques, especially flow cytometry that suffers from limited sensitivity and high false positive rates.

In alternative embodiments, fluorescent droplets that contain a target can be sorted in the 3D particle counting system using e.g., optical tweezer, optical trap and optical lattice. This enables the sorted target(s) to be further processed and analyzed.

The existing 1D on-chip droplet counting system (which is also used in the droplet digital PCR system) and other particle counting systems including flow cytometry suffer from low throughput: they typically operate at 1000 s particles s⁻¹ and are only able to analyze a total of 100,000 s to 1 million droplets (or a total sample volume of ˜tens of microliter).31, 34 Therefore, the existing droplet detection systems inevitably require sample preparation to purify and enrich targets and reduce sample volume before droplet encapsulation. In our system, however, we want to rapidly analyze unprocessed biological samples (e.g., blood) with a clinical sample volume of typically milliliters that translates up to billions of droplets. To effectively analyze these many droplets in a short period of time and detect single fluorescent, bacteria-containing droplets among millions of empty ones, in our invented Integrated Comprehensive Droplet Digital Detection (IC 3D) system, we integrated a 3D particle counter21 as we described earlier that can detect fluorescent particles from milliliter volumes at single-particle sensitivity within minutes.

FIG. 17 illustrates exemplary schematic diagrams of exemplary 3D particle counting systems of the invention. In alternative embodiments, a two-channel setup is used to allow simultaneous red and green fluorescence detection for the rapid quantification of the total number of particles.

In alternative embodiments, the apparatus comprises a small microscope that has a horizontal geometry and a mechanical sleeve that holds a cylindrical cuvette of diameter 1 cm. Two motors provide rotational and vertical motion of the cuvette. The software allows the rotational speed to be varied in the 10-1100 rpm range and the vertical speed in the 1-15 mm s⁻¹ range. The vertical and rotational motions are produced respectively by the Linear Actuator and a VEXTA stepping motor model PK233PB. These motors are connected to a stage holding the transparent cuvette containing the sample. The excitation light generated by lasers is focused at the volume of observation (see photo). The excitation focus is positioned inside the cuvette and relatively close to the wall of the cuvette, at a distance of about 1 mm from the wall. This distance can be adjusted so that detection of particles and analysis could be done even in highly scattering media. The excitation sources are two diode lasers emitting at 469 nm or at 532 nm. Thus, a particle fluoresces when in the volume of observation. The use of a confocal microscope in combination with simple mechanical motions of the sample container in front of the objective provides the means to move and analyze a sample containing particles through an observation region without requiring a complex optical system comprised of moveable optical components, such as translating optical sources, mirrors or photodetectors. The excitation light from the two lasers are combined in one path through a set of dichroic filters ZT532nbdc and Z470rdc and directed through a 20×0.4 NA air objective to the same volume of excitation. Fluorescence emitted from the sample is collected by the same objective, transmitted through the set of dichroic filters, focused by a lens into a large pinhole (diameter =2 mm), and then collimated by a second lens to the detectors. A dichroic beam splitter T5501pxr-25mmNR separates the emission beam into two light paths prior to its detection by two photomultiplier tubes (PMT). Two emission filters (FF01-HQ 500/24-25 and LP5600) are located in front of each PMT. The signal from the PMT is sent to the analog to digital converter (ADC) and to the acquisition card. The sampling frequency is set to 100,000 Hz, corresponding to a time resolution of 10 μs.

In alternative embodiments, the optics of the microscope is designed to measure a relatively large volume (100 pL) in about 0.01 ms. The rotation of the tube in a spiral motion for about 100 seconds allows us to effectively explore about 1 ml of the tube. When using this exemplary optical setup, the device is penetrating only 150 μm into the sample. Therefore, strongly scattering samples such as whole blood (even before dilution) that have a transmittance at 500 nm of about 10% for a 250 μm path length can be easily handled.

In alternative embodiments, the invention provides alternative designs of the exemplary IC 3D system of the invention (FIGS. 32 and 33) including an automated, portable device that permits multiplex and parallel analysis. In alternative embodiments, the device is comprised of multiple laser sources and detectors capable of reading at different wavelengths. The multiplex system can permit simultaneous reading of multiple sensors (labeled in different colors) that are coded for different pathogens. A carousel can also be added in our apparatus capable of accommodating multiple sample vials for carrying out parallel tests.

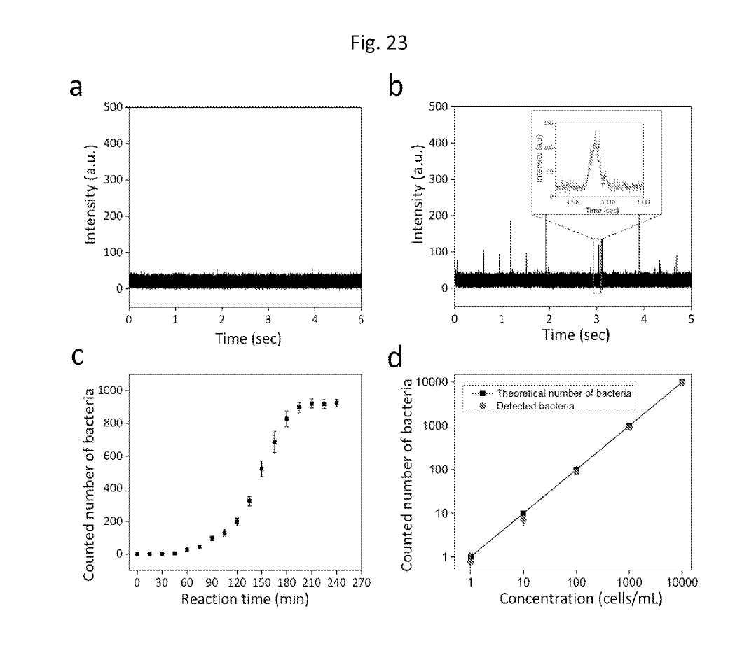

We encapsulated bacteria spiked blood and DNAzyme sensors into droplets as we described previously (see Example 1). Compartmentalization of target-specific reactions is a critical step to “light up” the droplet “reactors” that contain target bacteria so that they can be detected by the 3D particle counting system. Droplets were collected in a cuvette (FIG. 14d) and then analyzed by the 3D particle counting system. Using this system, we have demonstrated that fluorescent droplets that contain single target E. coli K12 and DNAzyme sensors can be detected at single-droplet sensitivity from a typical 2 ml sample volume within 3 minutes measurement time (FIG. 23a, b). Our current system typically operates at a throughput of ˜100,000 s droplets s⁻¹ or an effective volume of observation of ˜0.1 ml min⁻¹ With such a high throughput, the sample volume increase resulted from blood dilution in our experiments become less a problem. FIG. 23b shows a typical time trace with fluorescence intensity spikes obtained from bacterium-containing droplets.

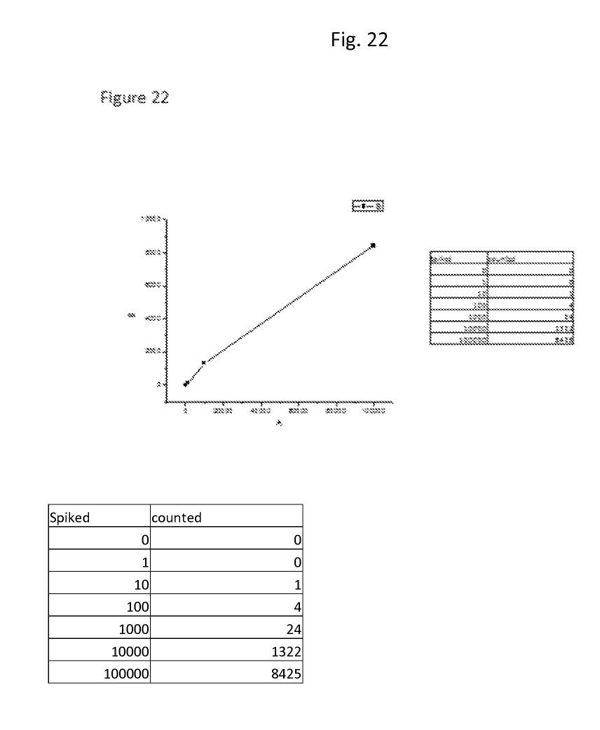

In alternative embodiments, the invention includes a pattern recognition algorithm (FIG. 23b inset box) and signal calibration (FIGS. 22 and 23d) for the IC 3D assay. In our IC 3D assay, the detection of a “hit” is defined by a pattern recognition algorithm (FIG. 23b, inset box) rather than threshold intensity (which is widely used in conventional 1D particle counting systems (e.g., BioRad ddPCR system) and typically suffers from higher false positive/negative rates because the intensity is dependent on many factors including lasers and detectors). Briefly, a fluorescent particle (droplet in our paper) is detected by the “shape” produced by the passage of the particle in the volume of illumination, which is Gaussian for our instrument. The pattern recognition implemented in the software SimFCS (Laboratory for Fluorescence Dynamics, Irvine, Calif., available at www.lfd.uci.edu/globals/) detects the time of the passage of the particle and the amplitude of the detected pattern. Predetermined using fluorescent droplets that contain DNAzyme sensors already reacted with bacteria (the “standard”), our pattern recognition algorithm can automatically filter the noise and only report true bacterium-containing, fluorescent droplets. Such pattern recognition allows us to achieve exceptionally reliable and accurate detection of a low concentration of fluorescent droplets in large sample volumes, which translates to essentially zero false-positive rate (i.e., a “hit” is always a true positive even among hundreds of millions of empty droplets). This is supported the 0 total count for control samples including healthy donor blood samples without bacteria (n=5) or spiked with non-target clinical bacterial isolates (n=8). In alternative embodiments, the invention provides a method to establish calibration curves for 3D particle counting system using fluorescent droplets that contain DNAzyme sensors already reacted with bacteria or FITC.

To determine the minimal DNAzyme reaction time that is required in our IC 3D system to detect bacteria in unprocessed blood, we monitored the signal from a 2 ml droplet solution over time using our 3D particle counter (FIG. 23c). We observed that, in as little as 45 min of DNAzyme reaction, the IC 3D test can generate a “yes or no” result while 3.5 hours is typically required to provide absolutely quantitative data about the number of cells in the sample.

We next demonstrate that our system can provide absolute quantification of target bacteria at a broad range of extremely low concentration from 1 to 10,000 bacteria ml⁻¹ with single-cell sensitivity and an exceptional limit of detection (LOD) in the single digit regime (FIG. 23d). There is exceptional linear correlation between the detected number of droplets and the actual concentration of targeted bacteria spiked in the blood sample. Regarding the false negative rate and analytical errors in these positive samples, for concentrations of 10-10,000 cells ml⁻¹, we are always able to detect target E. coli despite of the analytical errors, i.e., report as “positive” in a “yes or no” test, with essentially 0 false negative rate. For samples of 1 cell ml⁻¹, our assay typically detects the bacterium ˜77% of the time. Note that that the time of the measurement could be expanded to decrease the errors.^{20,21} Therefore, the LOD lies in the single digit regime.

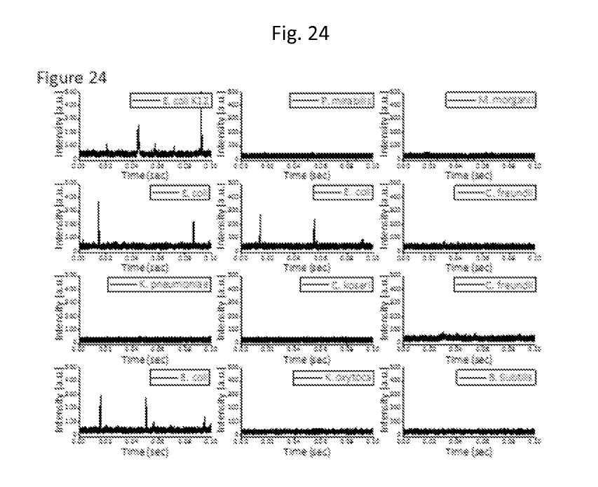

To demonstrate the potential clinical applicability, we tested our system using clinical bacterial isolates obtained from positive blood cultures. We found that our IC 3D system can selectively and robustly detect clinical E. coli isolates with a performance similar to what we observed for positive control E. coli K12 (FIG. 24).

In alternative embodiments, exemplary methods and systems of the invention comprising single-cell detection serve as a platform for the detection and screening for slowly-growing organisms (e.g., Mycobacterium tuberculosis).

In alternative embodiments, exemplary methods and systems of the invention serve as a platform technology where other types of sensors can be employed to selectively and sensitively detect almost any type of rare species in the blood including cells (e.g., bacteria, circulating tumor cells and stem cells), viruses, and other low abundant molecular targets.

In alternative embodiments, in addition to DNAzyme sensors, other sensing systems (e.g., digital PCR) for known target genes or molecules can also be integrated with our droplet microfluidics and 3D particle counting system for rapid single bacteria detection.

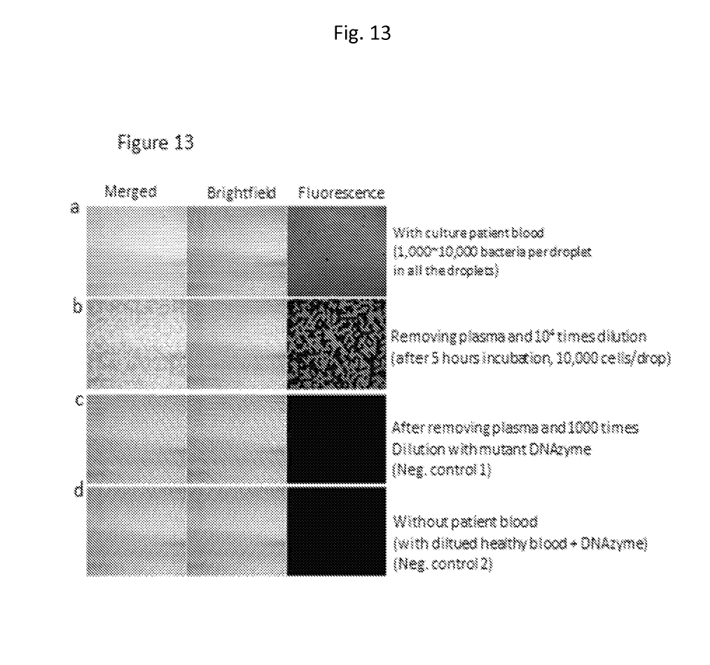

In alternative embodiments, target bacteria can be further cultured and proliferated in the droplets to amplify the signal before measurement (FIG. 13).

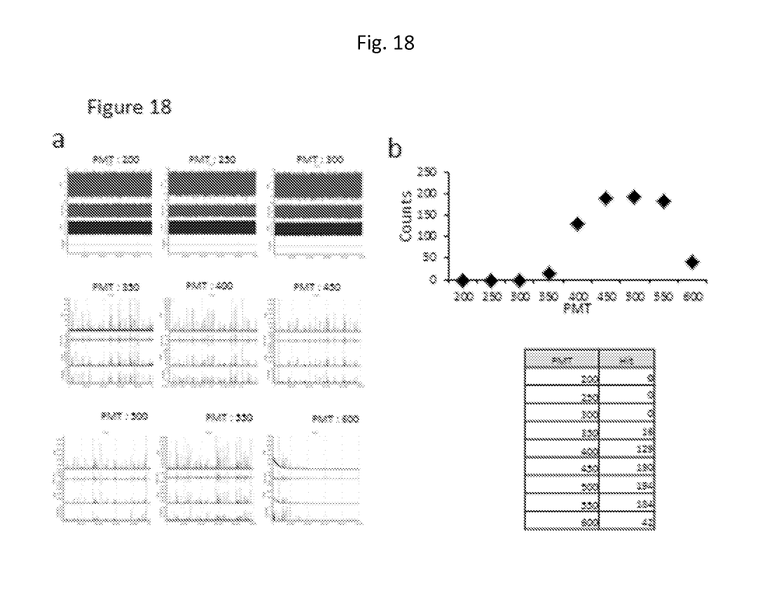

In alternative embodiments, a one or more parameters, including droplet size, reaction time, sensor concentration, fluorophore/quencher pair, blood dilution factor scanning time (1-10 min), RPM (200-1000) and PMT (photomultiplier tube) (200-800), can be optimized to achieve optimal performance (i.e., signal/background ratio, sensitivity, LOD and assay time), as illustrated e.g., in FIGS. 18, 19 and 20. The use of multi-color sensor system can further minimize false positive/negative rates. As smaller droplet sizes lead to higher target concentrations from single cells, increasing the signal/background ratio and decreasing the detection time, we specifically compare the performance of three different droplet sizes of 10, 20 and 50 μm. For the droplet assay, we can use various DNAzyme sensor concentrations (e.g., 10, 50, 100, 200 and 500 nM) to reach the best balance of detection time and signal/background ratio. Biological sample (e.g., blood) concentration after dilution can range from 5%-50%.

In alternative embodiments, this invention provides a fully integrated IC 3D system that is a bench-top, single-step, sample-to-result diagnostic consisting three major components (FIGS. 32 and 33) 1) bacterium-detecting DNAzyme sensors, 2) high throughput high efficiency (HT-HE) encapsulation system (FIG. 33a and b). For example, the cost effective modular microfluidic system that can accommodate up to 256 channels allows encapsulation of a 3 mL sample in <15 minutes, and 3) a 3D particle counter to rapidly measure small numbers of bacterium-containing fluorescent droplets from large volumes (FIG. 33c,d). In alternative embodiments, the invention includes (a) designs of the hardware in order to make it portable (that is of a smaller footprint and with the computer integrated with the instrument); (b) the integration of microfluidics components required for the formation of the droplets encapsulating the target bacteria; and (c) the improvement in the ergonomics in order to make the instrument usable by health care providers and technicians at large. This area encompasses the hardware design of the instrument and analysis software. To operate the system, sterile whole blood samples are mixed with DNAzyme sensor and bacterial lysis (lysozyme) solution and loaded into a pressure chamber. The PC-based control system will then pressurize the chambers and feed the sample and continuous phase oil into the droplet forming chips. The resulting droplets will then be collected in a cuvette and counted using the 3D particle counter. The data (i.e., the numbers of bacteria in the sample) will be processed by customized software and displayed on the computer screen. The system described above is rich in terms of future applications. Three lasers in the particle counter make it possible to simultaneously read three different sensors (and molecular targets). For example, CRE and ESBL sensors could be used together in a cocktail to determine whether individual bacteria in the sample contain one, the other or both resistance mechanisms, and the quantitative feature means that the absolute concentrations of each combination are recorded. A third sensor or a dye could be used as an internal quality or quantitation reference with its components added in known quantities through the instrument's reagents. While the assay is capable of handling up to milliliters of blood, the IC 3D sensitivity may allow even less to be used per assay than the 5-10 mLs typically drawn for traditional testing. This would open the door to running many more specific assays on a blood draw. On the other hand, if needed, the cuvette size could be increased to handle a larger volume of blood.

### Example 3

### Detect Antimicrobial Resistance by the IC 3D Using Fluorogenic Substrates

As a platform technology, the IC 3D system can integrate other sensing methods (e.g., enzymatic assays, PCR and isothermal signal amplifications) with droplet microfluidics and a 3D particle counter can serve as a platform for rapid detection and analysis of almost any type of low abundant markers in biological samples including cells (e.g., bacteria, circulating tumor cells and stem cells), extracellular vesicles (e.g., exosomes), viruses (e.g., HIV), and molecular markers (e.g., nucleic acids and proteins) (FIG. 1).

In alternative embodiments, the invention provides IC 3D tests for antimicrobial resistance using fluorogenic substrates³⁶ for beta-lactamases and carbapenemases, see e.g. FIG. 26. These tests allow us to quickly detect extended spectrum beta-lactamase (ESBL)-producing Enterobacteriaceae and carbapenem-resistant Enterobacteriaceae (CRE) that are among the most prevalent antimicrobial resistant pathogens.

### Example 4

### Microencapsulated Detection for Cancer, e.g. CTC, Exosome, Nucleic Acids, Proteins, Peptides, Carbohydrates, Lipids, Small Molecules, Metal Ions

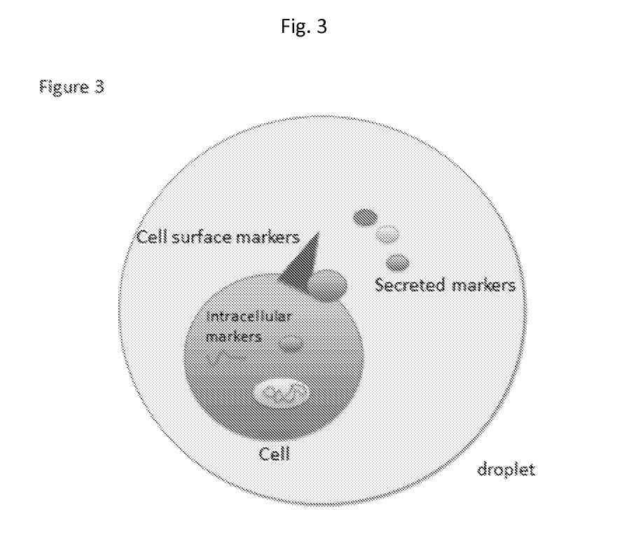

In alternative embodiments, the invention provides IC 3D test for routine detection and monitoring of cancer circulating tumor cells (CTCs), others markers and cancers e.g., nucleic acids, proteins, peptides, carbohydrates, lipids, small molecules, metal ions (FIGS. 3, 4 and 5), that is more efficient and robust than existing techniques.

For many carcinomas, e.g., breast cancer, over 90% of deaths are due to metastasis to distant organs. As metastasis is a multistep process in which disseminating cancer cells must survive transport through the systemic circulatory system, attention has recently been directed towards analysis and quantification of CTCs for early-stage diagnosis, prognosis and monitoring disease course. As CTCs are very rare (one CTC per 10⁷ leukocytes) and heterogeneous, traditional flow cytometry and immunological approaches (e.g., CELLSEARCH™ platform) are complex, expensive and time-consuming, and importantly, lack the sensitivity and specificity to reliably detect CTC in clinical settings. In alternative embodiments the invention provides a platform technology that selectively detects CTCs in non- or minimally processed patients' blood samples at single-cell sensitivity within minutes to hours. In alternative embodiments the invention provides a system that integrates novel fluorescent sensor technology, droplet microencapsulation and a 3D particle counter (i.e., the IC 3D). These sensors including e.g., DNA sensors are engineered to specifically react with the lysates of or intact target CTCs, leading to a rapid, real-time fluorescence signal. Patient samples (e.g., blood) can be mixed with sensor solution including cell lysis buffer within a microfluidic channel, which can be encapsulated in millions of individual picoliter droplets. While the invention is not limited by any mechanism of action, the confinement of CTCs in droplets significantly increases the concentration of target molecules (e.g., Her2 and EpCAM) that can be detected by the sensors in a rapid, real-time fashion. Therefore, the methods and systems of this invention represent a new paradigm in CTC detection which will potentially become a powerful tool for cancer diagnosis and prognosis, and monitoring disease progress and drug efficacy during therapy.

In alternative embodiments the invention provides microencapsulated sensor systems to detect rare cancer CTCs in clinical settings. In alternative embodiments, droplet microfluidics is integrated with sensors for rapid cancer CTC detection at single-cell sensitivity. In alternative embodiments, fluorogenic DNA sensors identified to specifically detect cancer biomarkers (e.g., Her2, EpCAM, CK19, and MUC1) are integrated with the droplet microfluidics system; where confinement of single CTC in droplets can significantly increase the sensitivity and shorten the detection time. Single-cell detection of CTCs from both buffer and spiked whole blood can be optimized.

To validate the ability of an exemplary device to detect CTCs from clinical specimens: patient blood specimens are used in correlation with patient diagnosis to determine the assay selectivity and specificity. Head-to-head comparisons are made using flow cytometry and CELLSEARCH™ platforms with respect to CTC detection selectivity, specificity and assay time.

The invention provides a platform technology that is suited for rapid and robust CTC detection and cancer e.g., breast cancer screening on a routine basis. In alternative embodiments, compositions, systems and methods of the invention are used for sequencing, personalized diagnostics and medicine, e.g., for detecting CTCs.

In alternative embodiments, compositions, systems and methods of the invention are used in genetic analysis, e.g., to detect a single cell gene or residue mutation, or to detect mRNA expression. In alternative embodiments, compositions, systems and methods of the invention are used to study and detect single cell heterogeneity based, e.g., on a gene or residue mutation or an mRNA expression level.

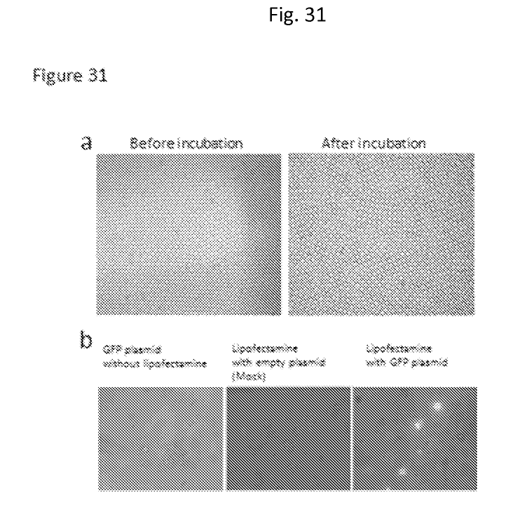

In alternative embodiments, cells are kept intact without lysis, which make it feasible for also using other tests or assays, e.g., such as immune staining or protein profiling. When used as “intact” cells while reagents (e.g., sensors, enzymes) can be delivered to the cell via viral or non-viral routes (e.g., transfection reagent, nanoparticles). In alternative embodiments, this invention includes a method to perform high throughput cell engineering at a single-cell level within the droplet. For instance, we have demonstrated that MCF7 cells can be encapsulated with transfection reagent containing GFP expression vector and engineered to express GFP (FIG. 31). In the case where cells are kept intact, it allows us to detect and analyze multiple types of markers simultaneously including intracellular, cell surface and secreted markers and correlate their expression and functions.

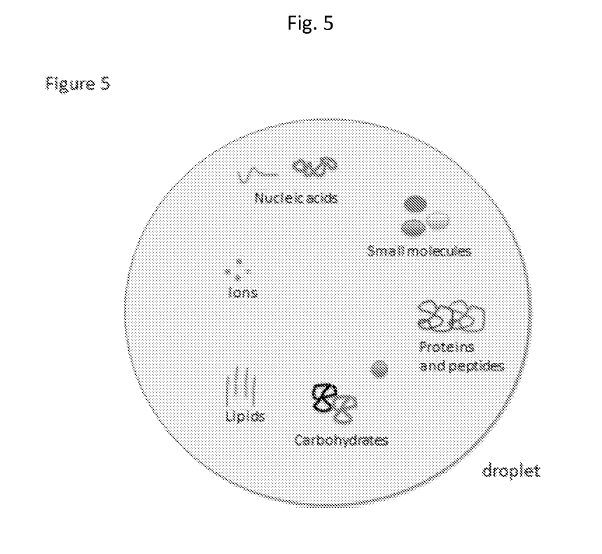

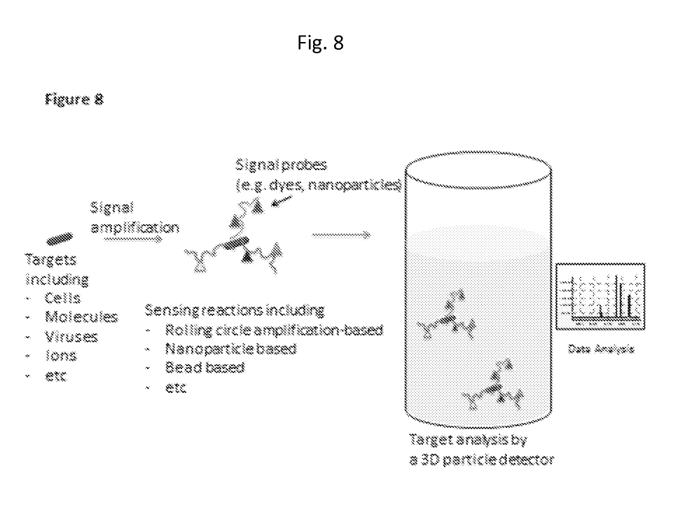

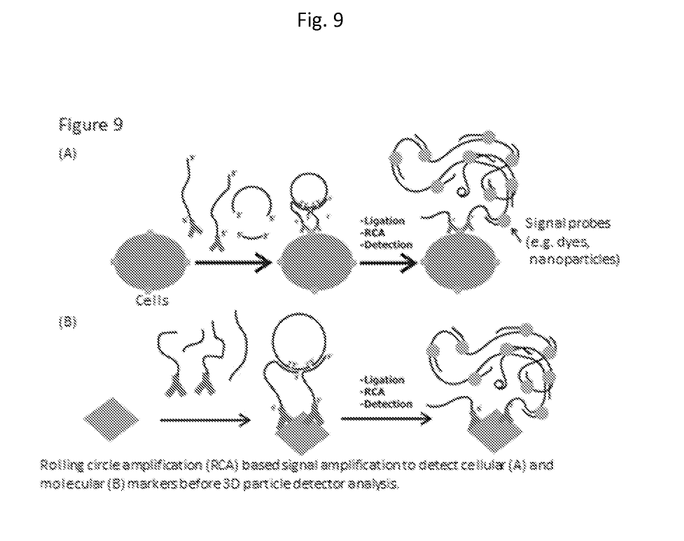

In alternative embodiments multiple enzyme reactions are used, which can give strong and high specific signal. In alternative embodiments, isothermal reactions including e.g. rolling circle amplification (RCA) reaction can be done in serum, facilitating direct CTCs detection in blood (see FIGS. 8 and 9 for example). In alternative embodiments, the invention provides systems and methods for detecting gene mutations and mRNA expression in a single cell level instead of, e.g., just using a surface marker (FIG. 5). In alternative embodiments, PNA openers and the like can be used to assist. Single cell genetic detection and sequencing assays, systems and methods of the invention provide a powerful new tool for personalized diagnostics and therapy.

In alternative embodiments, cancer cells, e.g., CTCs, can be characterized or detected by their cell surface, intracellular and secreted markers (see e.g., FIG. 3) or by mechanical properties. FIG. 3 schematically illustrates an exemplary method of the invention comprising detection of single cells and single cell markers including cell surface, intracellular and secreted markers, by exemplary integrated droplet encapsulation and 3D particle detector systems of the invention.

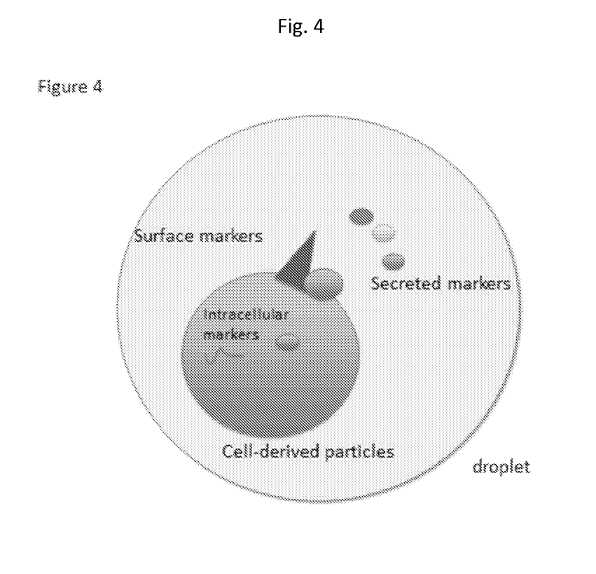

In alternative embodiments, cancer cells, e.g., CTCs, can be characterized or detected by detecting cancer markers, e.g., a cancer protein (e.g., Prostate-Specific Antigen (PSA), Her2, EpCAM, CK19, and MUC1), a cell-free nucleic acids (e.g., DNA, mRNA, miRNA and SNPs), a cell derived particles (e.g., exosomes, microvesicles, apoptotic bodies), lipids, carbohydrates, peptides, enzymes, small molecules and ions (FIGS. 4 and 5).

FIG. 4 schematically illustrates an exemplary method of the invention comprising detection of cell derived particles (e.g., exosomes, microvesicles, apoptotic bodies) and their markers by exemplary integrated droplet encapsulation and 3D particle detector systems of the invention.

FIG. 5 schematically illustrates an exemplary method of the invention comprising detection of cell-free markers including, but not limited to, nucleic acids, proteins, peptides, carbohydrates, lipids, small molecules, metal ions, etc by exemplary integrated droplet encapsulation and 3D particle detector systems of the invention.

In alternative embodiments, methods of the invention further comprise use (can be used in combination with) detection of cancer cells and markers by known assays, including nucleic acid based, antibody based, enzyme based, or chemical based, and the like. Biological samples can be first processed to reduce the volume and improve the purity by, for example, gradient centrifugation, washing, enrichment, cell lysis, magnetic bead capture and separation, and extraction, prior to the droplet encapsulation and subsequent analysis.

In alternative embodiments, methods of the invention includes detection, track, monitor single transplanted cells including e.g., stem cells and cancer stem cells. In alternative embodiments, the to-be-transplanted cells can be engineered with probes (e.g., enzymes, proteins) that can be secreted to blood or urine where they can be detected by the IC 3D. In alternative embodiments, the to-be-transplanted cells can be engineered with probes to be at downstream of a biological signaling event so the probes can only be activated and produced when a biological signaling event is turned on.

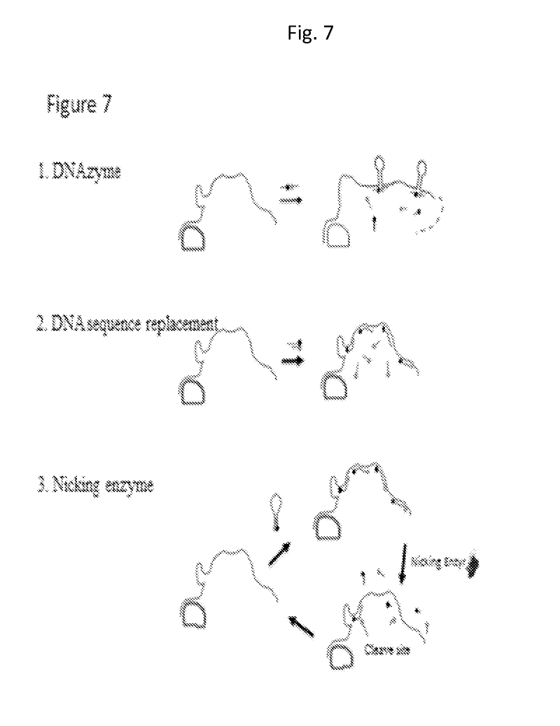

In alternative embodiments, methods of the invention further comprise detection of nucleic acids markers (both intracellular and cell-free circulating forms) including mRNA, DNA, miRNA, SNPs, and the like, which can be detected by PCR, RT-PCR, RCA, loop-mediated isothermal amplification (LAMP), nicking (e.g., EXPonential Amplification Reaction (EXPAR)), strand displacement, exponential isothermal amplification and hybridization, molecular beacons, aptamer, DNAzyme, or other real-time fluorescent sensors. In alternative embodiments, RCA combined with molecular beacon and nicking enzyme reactions can be used to detect nucleic acids markers and their mutations, see e.g., FIGS. 6 and 7.

FIG. 6 schematically illustrates an exemplary method of the invention comprising detection of nucleic acid mutations using padlock probe combined with nicking enzyme reaction in droplets. In alternative embodiments, methods of the invention further comprise an exemplary method of the invention comprising testing and optimization of AMPLIGASE™ (EPICENTRE, Madison, Wis.) ligation followed by nicking enzyme reaction for DNA mutation detection.

In alternative embodiments, methods of the invention further comprise an exemplary method of the invention comprising testing and optimization of AMPLIGASE™ (EPICENTRE, Madison, Wis.) ligation followed by nicking enzyme reaction for DNA mutation detection.

In alternative embodiments, methods of the invention further comprise an exemplary method of the invention comprising testing and optimization of T4 ligase ligation followed by nicking enzyme reaction.

In alternative embodiments, methods of the invention further comprise an exemplary method of the invention comprising testing and optimization of E.coli ligase ligation followed by nicking enzyme reaction.

In alternative embodiments, methods of the invention further comprise mRNA BRAF V600E mutation detection as an example. In such assays, following RCA reaction, signal can be amplified and produced by a variety of methods including DNAzyme based, strand displacement, or nicking enzymes.

Nucleic acid markers and mutations can also be detected by PCR and RT-PCR. For example, we have demonstrated the reactions of using PCR to detect BRAF V600E mutation and BRAF G464V, see, e.g., FIG. 27.

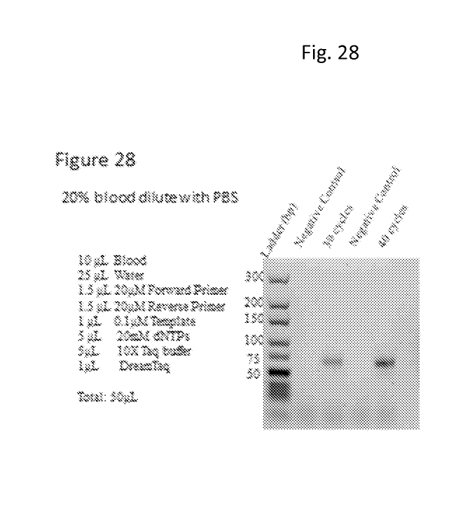

We have demonstrated PCR can be performed with plasma and blood samples, see, e.g., FIG. 28.

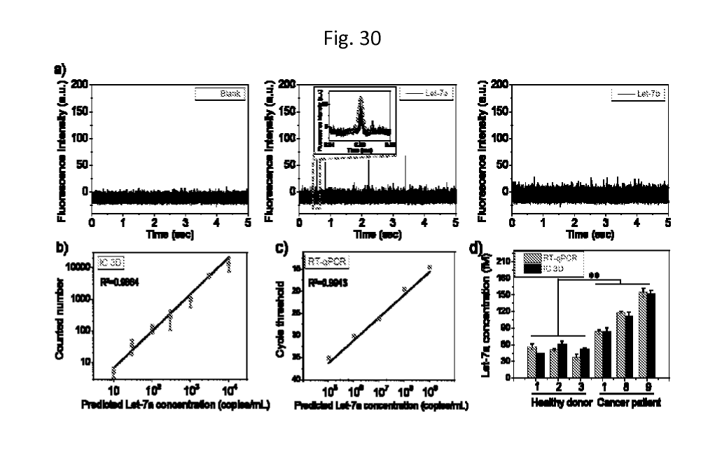

We have also demonstrated that Let-7a miRNA using exponential amplification reaction (EXPAR) by a combination of polymerase strand extension and single-strand nicking reactions, see e.g., FIG. 30. Briefly, circulating miRNAs are emerging biomarkers for a variety of diseases including cancer and neurological diseases. Analysis and quantification of miRNAs in blood can be potentially used for early detection, surveillance monitoring and drug response evaluation. Using Let-7a as a target, we demonstrate that IC 3D can precisely quantify target miRNA directly from plasma at extremely low concentrations ranging from 10 to 10,000 copies/mL in ≦2 hours. Using this new tool, we further demonstrate that target miRNA content in colon cancer patient samples is significantly higher than that in healthy donor samples. Our assay can also discriminate single-nucleotide differences between microRNAs within the same family with high specificity. More specifically, EXPonential Amplification Reaction (EXPAR) in droplet for miRNA detection was investigated. Droplet microfluidic device was designed and fabricated using standard soft lithography and operated as we described previously. 10% plasma samples and sensing reagents (DNA templates, DNA polymerase (Vent (exo-)), nicking endonuclease (Nt.BstNBI), EvaGreen and deoxyribonucleotides (dNTPs)) were mixed within microfluidic channel and then formed droplets with uniform sizes (30 μm diameter in this work) using flow-focusing mechanism. As EXPAR reaction has nonspecific background amplification if given sufficient time, to identify the optimal detection time that generates maximum target specific fluorescence signal with minimum background, we first studied EXPAR kinetics in droplets for single miRNA detection. We found that some droplets begin to light up in Let-7a sample around 40 min reaction. At 50 min, the number of fluorescent droplets in Let-7a sample increases to the predicted number (10 per 113 droplets at 10 fM bulk concentration) while Let-7b and blank samples still have few fluorescent droplets. However, at 60 min reaction, some non-specific signal begins to arise. This set of data allow us to 1) demonstrate the feasibility of single miRNA detection in droplets and 2) identify 50 min as the optimal EXPAR reaction time which was used in subsequent droplet measurement to best distinguish the target signal from nonspecific ones. FIG. 30a shows typical time trace with fluorescence intensity spikes obtained from Let-7a-containing droplets or controls. To extract the measurement of the concentration and/or brightness of the droplets in the sample, the temporal profile generated by the photodetector is analyzed with a pattern recognition algorithm (FIG. 30a, middle panel, inset box) implemented in the software SimFCS. The pattern recognition algorithm matches amplitude and shape features in the temporal profile to a predetermined pattern that is characteristic of the time-dependent fluorescence intensity of droplets passing through the observation volume. Such pattern recognition allows us to achieve exceptionally reliable and accurate detection of a low concentration of fluorescent droplets in large sample volumes. We next demonstrate that IC 3D can provide absolute quantification of target Let-7a at a broad range of extremely low concentration from ˜10 to 10,000 copies/mL with single-molecule sensitivity and a Limit of Detection (LOD) around 10 copies/mL (FIG. 30b). There is a linear correlation between the detected number of droplets and the actual concentration of targeted miRNA spiked in plasma sample. The LOD of the IC 3D assay is a few orders of magnitude lower than that of the current gold standard RT-qPCR, which is ˜10⁵ copies/mL (i.e. in the fM range) (FIG. 30c). Note also that RT-qPCR cannot operate directly using plasma samples and requires miRNA extraction and purification. To demonstrate the potential clinical applicability of IC 3D system, plasma samples from colon cancer patients and healthy donors were employed. The plasma samples were first tested in bulk using EXPAR and demonstrated that EXPAR can be used for direct Let-7a detection in 10% plasma although the fluorescent amplification curves between healthy donor and colon cancer patient samples cannot be well distinguished. Then IC 3D was used to measure Let-7a concentration in 3 representative colon cancer patient samples (or healthy donor controls) and demonstrate that the IC 3D can robustly quantify target miRNA content directly from plasma as validated by RT-qPCR for the same samples (FIG. 30d). RNase treated plasma was also included as a negative control to confirm that the fluorescent droplets are due to the target Let-7a. Interestingly, we found that the Let-7a content in colon cancer samples is statistically significantly higher than that in healthy donor samples as digitally quantified by IC 3D (which are not distinguishable by bulk EXPAR). The higher level of Let-7a (although known as a tumor suppressor) [17] in cancer samples could be due to the higher content of exosomes and miRNAs that shed from tumor into blood stream. [18]

In alternative embodiments, methods of the invention are used to detect protein markers (on cell surface or secreted), e.g., they can be detected by antibody-based ELISA, sandwich based, immunostaining, antibody capture, secondary antibody amplification, proximity ligation based, aptamer, DNAzyme, or other real-time fluorescent sensors.

In alternative embodiments, methods of the invention are used to detect cell surface or free protein markers, for example, detecting EpCAM and Her2, e.g., by standard proximity ligation based assays that can be followed by signal amplification. In alternative embodiments, PSA can be detected by a real-time DNA sensor, or using fluorogenic substrates.

### Example 5

### Detection and Analysis of Cells or Biological Markers Using a 3D Particle Counter Without Droplets

In alternative embodiments, the invention provides rapid and sensitive systems or methods for detecting a biological, a physiological or a pathological maker, or a single molecule or a single cell using a target detection process with and without signal amplification integrated directly with a 3D particle detector (FIG. 8), comprising:

Features:

Our systems possess the following unique features that cannot be easily achieved by traditional detection assays:

1) Low abundance markers (e.g., 1-1 million/mL)

2) Able to interrogate large sample volume (μLs to mLs) and high throughput

3) Rapid (minutes to hours)

4) Broad detection range

5) Multiplexable

6) No or minimal sample preparation is required.

Samples:

1) wherein the biological sample comprises a blood, serum, saliva, tear, stool, urine or CSF sample from a patient

2) wherein the samples are obtained from food, water and air.

Sample Preparation

The samples can be directly assayed with no or minimal (e.g., dilution) processing.

Standard, established biological sample preparation processes including dilution, purification, enrichment, extraction, centrifugation, cell lysis, magnetic bead assays, and washing steps, although not required, can be integrated into the invented assays.

Targets:

The target species that can be detected and analyzed by the invented systems include, but not limited to (FIG. 8):

Cells (e.g., cancer cell, stem/progenitor cell, immune cell), pathogens (e.g., bacteria, multi-drug resistant organisms (MDRO), tuberculosis (TB)), viruses (e.g., HIV), cell-derived vesicles (e.g., exosome, microvesicles, apoptotic bodies), nucleic acids (e.g., SNPs, mutations, expression), proteins (e.g., PSA), enzymes (e.g., MMPs), peptides, lipids, carbohydrates, polysaccharides, small molecules or metal ions.

The forms of target species include cell surface (e.g., EpCAM, N-cadherin, CD44, CD24), intracellular, and secreted markers (cell secretome), cell free circulating markers (e.g., miRNA, DNA, protein markers), metabolic markers, mechanical markers (e.g. cell deformability, stiffness, cytoskeleton, etc).

In addition of the expression, the invented systems can also be used to detect or monitor a biological event, e.g. DNA hybridization, protein receptor-ligand interaction, enzyme-substrate interaction, and cell surface receptor dimerization (including both homo and hetero-clustering), co-localization, or interaction with soluble ligands and drugs and another cells.

Target Detection Assays

There are a wide variety of established fluorescence bioassays that can be utilized in our system to selectively detect the targets for 3D particle counter analysis. Such assays include, both not limited to, (FIG. 8). Nucleic acid based, antibody based, enzyme based, chemical based, nanoparticle-based, bead-based or used in combination, etc.

Some more specific examples are given below:

Nucleic acid based assays including hybridization, molecular beacons, aptamer, DNAzyme, or other real-time fluorescent sensors.

Antibody-based assay include ELISA, sandwich based, immunostaining, antibody capture, secondary antibody amplification, or proximity ligation based.
Enzyme based assays include PCR, RT-PCR, RCA, loop-mediated isothermal amplification (LAMP), nicking, strand displacement, and exponential isothermal amplification (Lab Chip, 2012, 12, 2469-2486) In some cases, the target itself such as PSA or MMPs can serve as enzyme to trigger a detection process.

In RCA-based detection, the target recognition binder is a biological or chemical moiety including aptamer or antibody. RCA can be a linear or branched (i.e., exponential amplification). RCA products can be loaded, stained and analyzed by dyes, nanoparticle or quantum dots.

3D Particle Counter

3D particle counter can be an instrument system as shown in FIG. 17 or a portable system for point-of-care applications.

Integrated Exemplary Systems of the Invention

Our systems can be engineered with desirable portability, automating fluid handling, and integrating electronics including a diode laser (light source), APD (detector), Operating (vinci, ISS Inc.) & data analyzing software (SimFCS), display, with a 3D particle counting system. This envisioned device can also be integrated with multiple disposable microfluidic “cartridges,” permitting multiplex and rapid detection of multiple types of targets simultaneously. The device can be fully automated, and can be fabricated as an all-in-one system or with modular components. It can also be linked to smart phone and bluetooth etc for point-of-care applications (FIGS. 32 and 33).

Applications

The invention's novel approach of target detection process (with or without signal amplification) and the 3D particle counter system is innovative and powerful: it permits selective detection of target species in biological samples in mL volume within minutes that is currently not possible. Therefore, we believe our technology has the potential to revolutionize how we detect and analyze low concentration biological particles and markers and can be utilized in a large variety of detection bioanalysis and diagnosis applications including, but not limited to:

Infectious diseases Pathogens (bacteria, viruses, fungi, etc). Skin infection, wound, diabetic ulcer, HIV, bacteria, TB, MDROs (e.g. MRSA)

Cancer

Diabetes

Alzheimer disease (e.g., Amyloid beta, Tau proteins)

Inflammatory and autoimmune diseases (e.g., CD4 T cell, immune cell count)

Stem cell and regenerative medicine (e.g., mesenchymal stromal cells, endothelial progenitor cells, hematopoietic stem cells, or the cells can be endogenous or exogenously transplanted cells)

Cardiovascular diseases (e.g., C-Reactive Protein (CRP), B-type natriuretic peptide (BNP), troponin, Cystatin C, IL-6)

Drug and abuse (e.g. Tetrahydrocannabinol, THC)

Newborn screening

The system can also be used to study new biology, cell-drug interactions and drug susceptibility, to develop new drugs and therapeutics and monitor disease progress and treatment efficacy or used as companion diagnostics, and to be used in sequencing, personalized diagnostics and medicine.

In addition to medical applications, our system can also be used for other areas including food industry, agriculture, water systems, air systems, and defense applications.

Rolling Circle Amplification Coupled Detection with 3D Particle Counter:

In alternative embodiments, this invention includes a novel detection system that integrates rolling circle amplification (RCA) and a 3D particle counter (FIG. 9). RCA is a simple and efficient isothermal enzymatic process that utilized unique DNA and RNA polymerases (Phi29, Bst, and Vent exo-DNA polymerase for DNA, and T7 RNA polymerase for RNA) to generate long single stranded DNA (ssDNA) and RNA (Rolling Circle Amplification: A Versatile Tool for Chemical Biology, Materials Science and Medicine, Ali, et al. Chem. Soc. Rev, DOI:10.1039/C3CS60439J.). RCA can be used to detect a variety of targets including DNA, RNA, DNA methylation, SNP, small molecules, proteins, and cells. RCA can be performed in a linear or hyperbranched exponential fashion. RCA products can be modulated to have different lengths, sizes, sequences and structures. RCA products can be loaded, stained and analyzed by dyes, probes, nanoparticles or quantum dots. Biological markers (e.g., cells, vesicles and molecules) can be detected and amplified by RCA (for example through proximity ligation based methods as shown in FIG. 9) and then can analyzed and detected by 3D particle counter.

Cancer Cell Detection Using 3D Particle Counter:

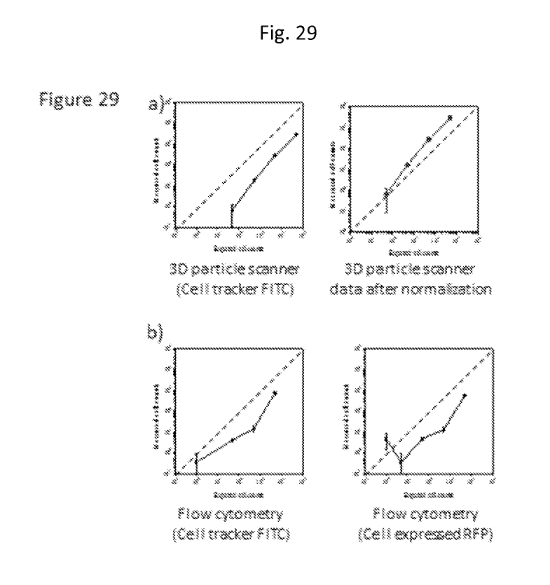

Cells, for example cancer cells, in biological samples can be stained, processed and directed detected by 3D particle counter (FIG. 29a) which is much more efficient than traditional assays including flow cytometry (FIG. 29b) regarding detection sensitivity and detection limit.

### Example 6

### In vitro Evolution to Generate Cancer-Specific DNAzyme Sensors

The following describes an exemplary method of the invention comprising in vitro evolution to generate cancer-specific DNAzyme sensors.

The invention provides a technology that exploits powerful in vitro evolution to generate reliable, DNAzyme sensor-based cancer diagnostics, as illustrated in FIG. 34. In alternative embodiments multiple rounds of enrichment using cancer and normal blood samples as positive and negative selection targets, respectively, can identify DNAzyme sensors that specifically recognize a vital (or a unique panel of) molecular signature(s) that discriminate cancer from normal samples or other diseases that have related symptoms.

FIG. 34 illustrates an exemplary scheme of the invention for in vitro evolution of DNAzyme sensors for e.g., cancer diagnostics: a) Envisioned mix-and-read, DNAzyme sensor cancer diagnostics and their applications. b) Mechanism of DNAzyme sensor: it generates fluorescent signal upon interaction with the target (F is a Fluorescein-dT. R is ribonucleotide and Q denotes a dabcyl-dT). c) Schematic illustration of the in vitro selection process. First, the random DNA library is ligated to the substrate and incubated with normal serum to remove any non-specific sequences from the library pool. The un-cleaved sequences are purified and applied to the positive selection using the cancer serum. The cleaved molecules by the cancer serum are purified and amplified by PCR. After purification, the population is ligated to the substrate and applied to the next round of selection.

In alternative embodiments, the methods and systems of the invention can be used in the clinic to detect almost any kind of cancer (FIG. 34a). Such simple and inexpensive blood-tests, which are currently not available, can be easily incorporated into a routine physical checkup to screen for cancerous activities before presentation of overt symptoms. Such early intervention will therefore significantly increase the chances to treat cancer and reduce mortality. These exemplary assays of the invention, which are capable of reporting cancer progression during treatment and monitoring drug efficacy and safety, can be tools for therapy guidance and drug discovery. Thus, practicing the methods and systems of the invention can increase patient survival, improve quality of life and decrease the financial costs associated with patient care.

In alternative embodiments, the methods and systems of the invention for e.g. cancer sensor screening have many innovative features compared to current technologies (e.g., proteomic biomarker technology). The combination of powerful in vitro selection techniques and targeting the complex cancerous sera as a whole allows us to develop generic and reliable diagnostics without the need for identification of any specific disease biomarkers. The activator of a given DNAzyme can be a protein, a nucleic acid, a small molecule, or metal ions, etc. This is particularly advantageous as it allows us to bypass the tedious process of purifying the target molecules from extremely complex mixtures for developing detection methods: i.e., once isolated, the DNAzyme sensors can be immediately used for cancer detection. The multiple rounds of enrichment and amplification necessary for identification of DNAzyme sensors not only minimizes the high rates of false positive and negative results inherent in traditional methods of biomarker discovery (e.g. 2D gel electrophoresis coupled with MS)¹⁻³ but also allow us to identify the modest differences existing between some cancers and normal tissue. We can also mix multiple patients' serum samples together as the target in order to bypass the non-specific heterogeneity between patients, and therefore truly identify the molecular differences that uniquely discriminate cancer and normal samples. Additionally, our system has the potential to generate multiple DNAzyme sensors simultaneously in the same enriched library pool that respond to a panel of molecular signatures that collectively detect cancer with significantly higher sensitivity and specificity than other single biomarker based assays. Finally, our resultant assay has many appealing features, one of which is its inherently rapid, real-time, mix-and-read nature, which is ideal for rapid screening and monitoring of cancers on a routine basis.

In alternative embodiments, DNAzyme sensors can be optimized towards optimal performance, e.g., signal/background ratio and stability, for e.g., working in whole blood. In alternative embodiments, the invention provides blood-based diagnostics to distinguish established cancer cases from healthy controls with respect to sensitivity and specificity. Retrospective and longitudinal studies can be performed to further validate and test an assay performance in correlation to standard clinical diagnosis and blood tests (for example, ELISA for potential protein biomarkers found in the literature). DNAzyme sensor sensitivity and specificity can be optimized by an iterative, re-selection process.

In vitro Evolution.

Library design. A DNA library containing approximately 10¹⁴ random sequences is used for isolating DNAzyme sensors. As illustrated in FIG. 34c, the library consists of a variable region (blue color) of 40 nucleotides that is ligated to the fluorogenic, DNA-RNA chimeric substrate.¹⁰ The substrate contains a single ribonucleotide (riboadenosine) as a cleavage site that is flanked by a fluorophore (Fluorescein-dT) and a quencher (Dabcyl-dT) on each side. The rational is that specific DNA sequences in the library (i.e., DNAzymes) exist and cleave the ribonucleotide linkage, therefore producing a fluorescence signal, only in the presence of target patient blood. The random domain and substrate can be ligated using T4 DNA ligase following our previous protocol. Note that fixed sequence domains in the 5′ and 3′ ends of the library are incorporated as forward and reverse PCR primer binding sites, respectively.¹⁰ Library and all other oligonucleotides are purified by gel electrophoresis before use.

Positive and negative targets. Non-small cell lung cancer (NSCLC) was used as a model system because of its high mortality and urgent demand for early-stage diagnostics.¹⁻³ Age- and gender-matched, nonsmoking healthy donor samples will be obtained. We choose to mix multiple patients' samples together in order to minimize non-specific variation between patients and preanalytical variability, and therefore only select the DNAzyme sensors that are universal (for same stage/type of cancer) and specific (between cancer patients and healthy donors). To avoid blood type antigen incompatibility, serum samples are used in the selection process. Mixing serum samples is commonly used in biomarker discovery and does not produce adverse effects (i.e., no immunogenic response is observed).³⁸ Specifically, 10 NSCLC patient serum samples (0.5 ml each) (or healthy control serum samples) are mixed thoroughly, aliquoted, stored at −80° C., and used throughout in the entire selection process.

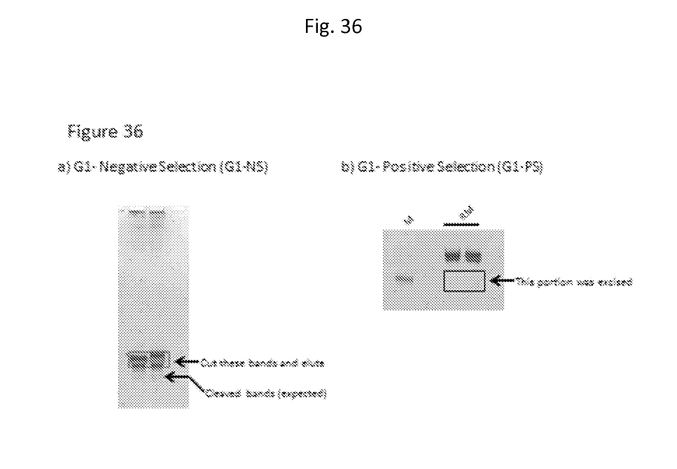

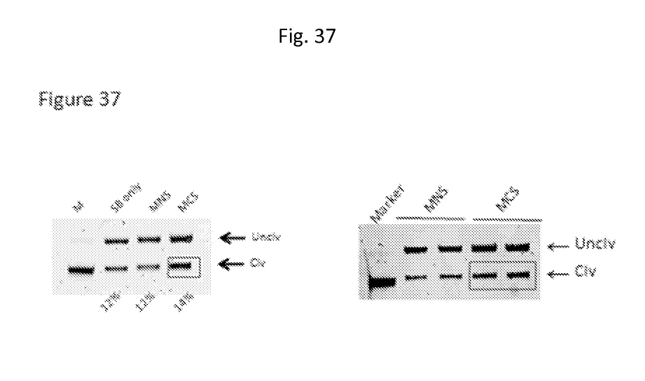

Selection. As illustrated in FIG. 34c, in vitro selection can be started by incubating the starting library (FIG. 35) (1 nmol) with healthy donor sera (200 μl) (negative selection) to remove nonspecific DNAzymes that are self-cleaving in the absence of target molecules or cleave in the presence of nonspecific molecules in the blood that are universal for all individuals (e.g., metal ions, ATP, albumin) Negative selection can be performed in the selection buffer (50 mM HEPES, 150 mM NaCl, 15 mM MgCl₂, 0.01% Tween 20, pH 7.5) for 3 hours providing sufficient time to remove all nonspecific DNAzymes. Ethanol precipitation can be performed to recover the library, and the uncleaved sequences are purified by gel electrophoresis (see FIGS. 36 and 37 for examples). Note that the cleaved and noncleaved molecules (which both are labeled with dyes) can be easily distinguished on the gel because of their different sizes. The purified noncleaved molecules can be incubated with cancerous serum mixture (positive selection) for only 10 min. This short incubation time in the positive selection allows us to only identify the DNAzyme sequences that respond rapidly to the target therefore reducing the assay time for cancer detection; indeed, the versatility of in vitro selection allows us to tailor the stringency of selection criteria to generate molecules with desirable properties.^{13,14} After positive selection, the cleaved molecules are ethanol precipitated and gel isolated. These isolated sequences can be amplified by primer-specific PCR, purified by gel electrophoresis, ligated to the substrate and then used in the second round of selection. In our experience, cleaved DNA bands become detectable after between 5-8 rounds, and 8 to 15 rounds of selection are typically needed for the completion of selection (i.e., no further significant increase of signal of cleaved DNA band).¹⁰ Finally, the final round of the DNA pool can be cloned into bacteria using a TA cloning kit (Fermentas), and at least 200 clones will be sent for sequencing (Functional Bioscience, Wisconsin).¹⁰

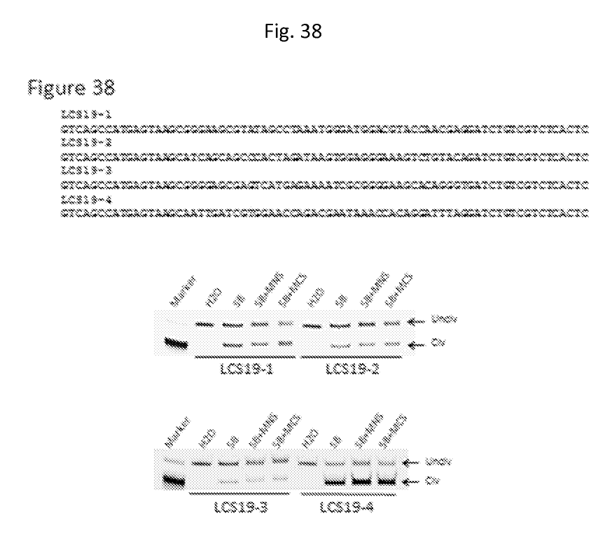

Using this approach, we obtained 19 classes of DNAzyme sensors that exhibited consistently higher activity in NSCLC samples than in healthy donor sera (see FIG. 38 for a set of selected sequences for analysis).

Characterize and engineer DNAzyme sequences towards optimal performance in blood. The identified DNAzyme sequences can be validated individually to make sure they are indeed capable of cleaving the substrate in the presence of target cancer but not normal sera. Additionally, while sera are used as the target during selection, clinical assays can also be performed using whole blood without any processing (i.e., mix-and-read). Therefore, we can characterize and modify identified DNAzyme sensors towards optimal performance with respect to signal/background ratio and stability in whole blood before we validate them clinically as cancer diagnostics.

Sequence performance analysis. In our experience, in vitro selection typically leads to 5-20 different classes (clones) of sequences.¹⁰ We can synthesize a representative sequence from each class from IDT. Each sequence can be tested for cleavage performance in the mixed cancerous patient and healthy sera separately. Two parameters, specificity (fluorescence signal ratio between cancer and normal sera) and kinetics (% of cleavage over time) will be studied. Specifically, the cleavage reactions can be conducted in a 96-well plate in 100 μL serum sample mixed in the selection buffer containing 100 nM DNAzyme sensors, and the cleavage activity can be monitored by plate reader based on the fluorescence signal enhancement in real-time. To further prove whether the signal is indeed due the cleavage at the cleavage site, the reaction mixtures can be analyzed by polyacrylamide gel electrophoresis. Because we hypothesize that in vitro selection may identify multiple DNAzyme sequences that define a unique panel of cancer biomarkers, we will carry forward all the sequences that meet the following criteria: 1) fluorescence signal ratio between cancer and normal sera >3, and 2) >50% molecules are cleaved in 1 h. The molecules that meet the above criteria will be combined and carried forward as a homogenous sensing solution in the following tasks.

Signal/background ratio of DNAzyme sensors in blood. The nature of our DNAzyme sensor (i.e., fluorophore and quencher are placed in close proximity and separated before and after adding target) warrants an extremely low background in the absence of target, but high signal in the presence of target.¹⁰ We typically obtain DNAzyme sensors that possess a signal/background ratio of >6-10 in buffer.¹⁰ When used in blood however, the autofluorescence of blood and interference of dyes (e.g., quenching) from the complex environment in the blood may compromise the signal/background ratio. Fluorescein and Dabcyl are initially chosen as fluorophore and quencher respectively in the selection process because of their simplicity, low-cost and the fact that the cleavage event is monitored by gel during selection. However, fluorescein/Dabcyl may not be ideal for using in the blood due to above-mentioned reasons. In this set of experiments, the fluorophore-quencher pairs including Cy3/BHQ2, Alexa 647/QSY21, TAMRA/BHQ2, Texas red/BHQ2 and Alexa 546/QSY9 (Glen research) are optimized to identify the one that is compatible with fluorescence detection in blood (i.e., not interfered with by blood autofluorescence) and reproducibly produces the highest signal/background ratio (i.e., >5).

Stability of DNAzyme sensors in blood. Since the DNAzymes are evolved directly in serum, we expect that they will be nuclease-resistant and stable in blood for at least the amount of time (i.e., 10 min) we use for selection. We can chemically modify the termini or backbone of DNAzymes (i.e., inverted T and phosphorothioates) which are established to increase the half-life of nucleic acids to up to hours or days in blood without compromising their functions.¹⁵ Alternatively, to protect the degradation of RNA linkage in the DNAzyme sensor, we can also include RNase inhibitor (ribolock, Fermentas) in the assay buffer.

Validate DNAzyme sensor specificity and selectivity across all stages of NSCLC. The isolated and optimized DNAzyme sensors can be tested for whether they are able to distinguish between people with NSCLC and healthy controls. Again, blood samples from established NSCLC patients at different stages are obtained and each sample is analyzed in triplicate with a numerical value of fluorescence for each sample before and after addition of the DNAzyme determined with a fluorescence plate reader. Samples can be normalized to background and analyzed to determine 1) specificity, 2) selectivity, and 3) response across different stages of NSCLC. DNAzymes can detect early (Stage 1) NSCLC for early detection of NSCLC. For all samples, head-to-head comparison can be made with ELISAs for carcinoembryonic antigen (CEA) and cytokeratin 19 fragment

(CYFRA 21-1), two biomarkers previously established as relatively sensitive and specific for NSCLC, although not fully clinically validated.^{1,2} Significance of experimental results can be determined with T-test.

In alternative embodiments, in practicing the invention, a re-selection component in the DNAzyme sensor development is integrated in order to optimize the properties of the DNAzyme sensors (i.e., 90% for both sensitivity and specificity). Re-selection is a process whereby the identified DNA sequence is partially randomized to provide the starting library for a new selection process where more stringent selection criteria will be enforced.¹⁵ Re-selection operates more efficiently with fewer rounds required than the first selection to generate desirable molecules. Indeed, re-selection has been used to improve the sensitivity and specificity of DNAzymes.¹⁵

If the sensitivity and specificity of our DNAzyme assays do not meet the 90% criteria in clinical tests, a re-selection process can be performed whereby DNAzyme sequences identified are partially randomized (30% mutation at each base position; for example if the original base is A, it will be kept 70% A, 10% each of C, T, and G), and chemically synthesized by IDT. The in vitro selection procedure is repeated as described above, except that more stringent and selective positive and negative selection targets are used. For instance, the group of patent samples that failed to be detected by initial DNAzyme sensors are segregated and used as the target for selection. In order to more effectively discriminate between cancer patients at different stages, one of them is used as the negative selection target for the other instead of using the healthy donor. The optimization using re-selection can allow selection of DNAzyme sensors that are universal (for same stage/type of cancer) and specific (between cancer, healthy donors or other disorders that share similar symptoms (e.g., lung inflammation), and between cancer at different stages).

Thus, the invention provides methods for making optimized DNAzyme sensors for sensitivity and selectivity (both >90%). DNAzyme sensors of the invention can be used as screening tools to identify patients at high risk of cancers at earlier stages than existing technologies. To definitively confirm and stage cancer, other traditional diagnosis tools, especially imaging techniques including CT and MRI can be used following our screening assays.

### Example 7

### Droplet Based Drug or Aptamer Screening

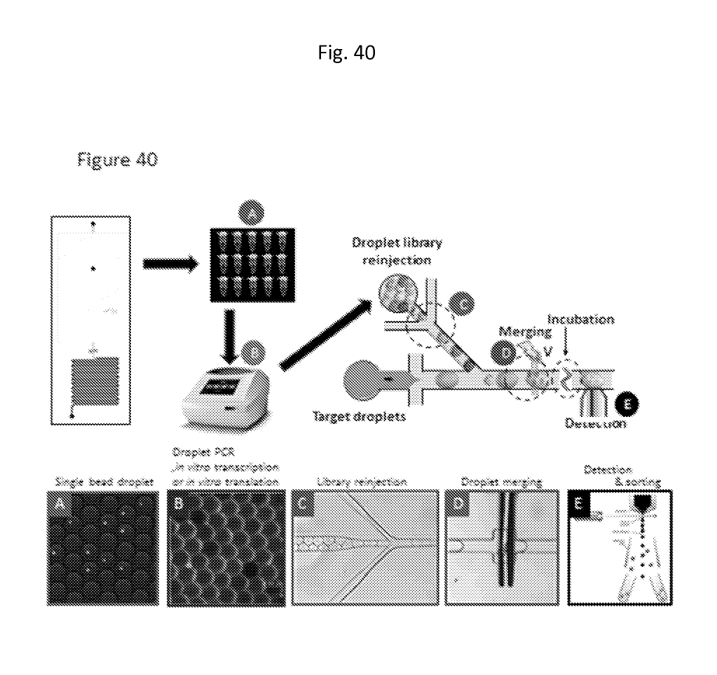

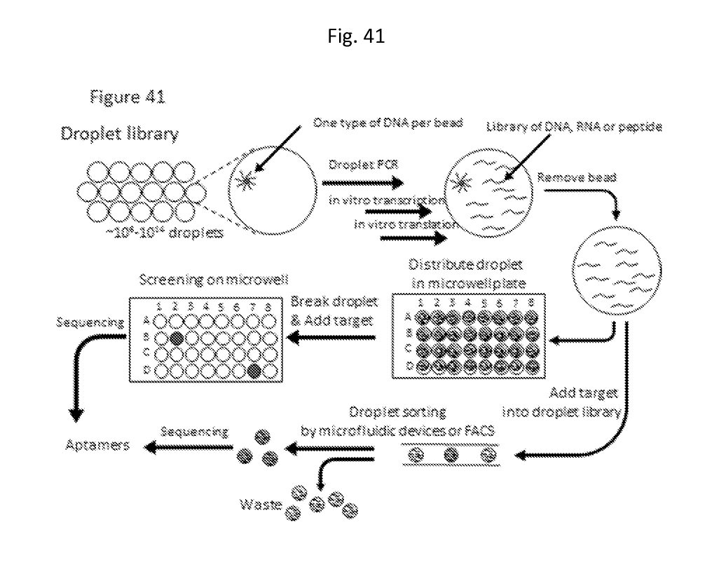

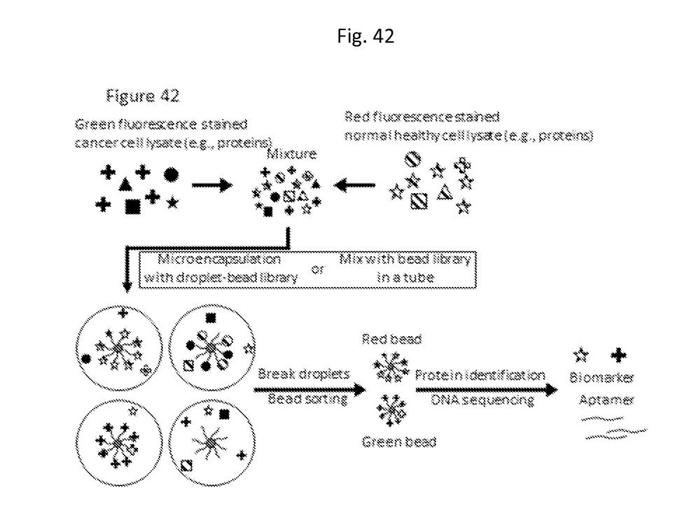

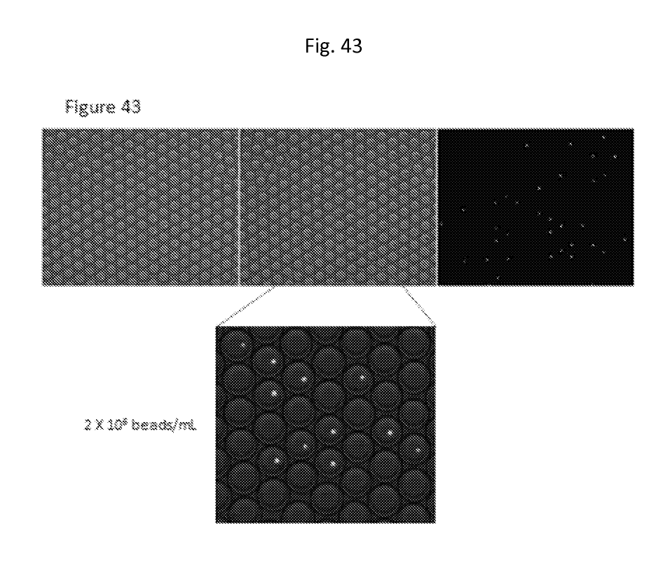

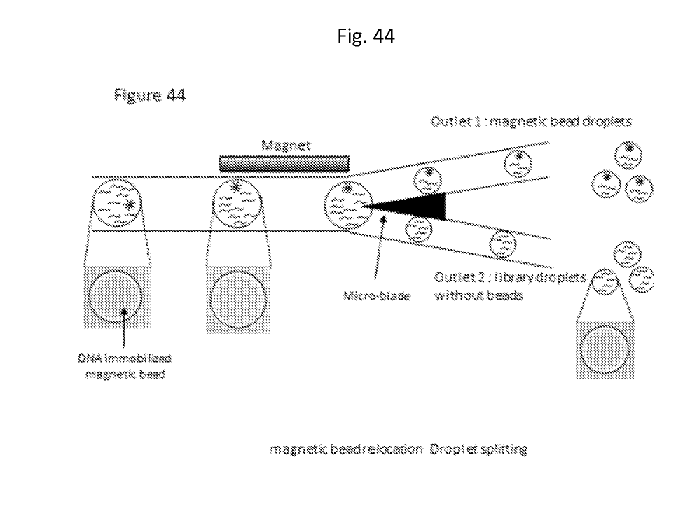

In alternative embodiments, we developed a drug screening and in vitro selection platform based on one type of molecule one droplet strategy, e.g., FIGS. 39 to 46. Confining reactions and screening in picoliter microdroplets allows efficient, high throughput, easy, inexpensive, and rapid screening. Microdroplets can be used for the library system for various molecules. Each droplet contains each DNA, RNA, or peptide after DNA amplification, transcription and translation, respectively. In an example, we synthesized DNA, RNA and peptide in droplet library containing e.g. approximately 2×10¹¹ different sequences in diversity using one bead one compound approach (FIG. 39). We encapsulated in picoliter droplets (20 μm in diameter) synthesized DNA on microbeads (FIG. 43). The on-bead DNA was amplified by PCR to generate a droplet DNA library. These DNA can then be transcribed and translated within the droplets to form RNA and peptide libraries (FIGS. 39, 40, and 41). In alternative embodiments, one type of molecule one droplet can be obtained through single molecule PCR in the droplet. In particular, the identity/sequence of translated proteins/peptides can be barcoded in the same droplet using the nucleic acid sequences, which provides a powerful tool for subsequent screening. In alternative embodiments, the droplets can be manipulated or processed including e.g., droplet merging, splitting, incubation, reinjection, imaging, analysis and sorting (FIGS. 40 and 44 for example). These DNA, RNA or peptide libraries can be used to screen in a variety of assays including e.g., protein-protein interaction, enzyme substrate interaction, receptor-ligand interaction, antibody-antigen interaction, ligand-cell binding, aptamer-target binding, aptamer-cell binding, DNAzyme reaction (see FIGS. 41, 45 and 46 for examples). These DNA, RNA or peptide libraries can also be used in evolution experiments to generate e.g., new enzymes or in screening and developing new biomarkers (FIG. 42). In alternative embodiments, the droplets can be sorted directly to identify the target-containing droplets. In alternative embodiments, droplets can be broken and target bound particles can then be sorted and analyzed. In alternative embodiments, as shown in FIG. 41, droplets can be distributed into microwell arrays where they can be kept intact or broken for further analysis, sorting, or printing to a new substrate (Biyani, et al. Microintaglio Printing of In situ Synthesized Proteins Enables Rapid Printing of High-Density Protein Microarrays Directly from DNA Microarrays, 2013 Appl. Phys. Express 6 087001; Biomolecule assay chip U.S. Pat. No. 8,592,348 B2). These facile, inexpensive exemplary libraries generated by methods and systems of the invention are valuable to screen and/or to obtain active biologics, such as therapeutics or diagnostics, and for biomarker discovery purposes.

### Example 8

### ENcapsulated ScreeNing of Aptamers by Reporter Amplification (ENSNARA)

In alternative embodiments, this invention presents an exemplary method termed “ENcapsulated ScreeNing of Aptamers by Reporter Amplification (ENSNARA)” for aptamer screening. As shown in FIGS. 47 and 48, in one embodiment, structure-switching aptamers can be identified using ENSNARA by employing allosteric control over a reporter enzyme in the droplet. ENSNARA can quickly generate aptamers for many targets which can be used immediately as real-time sensors.

In alternative embodiment, an exemplary allosteric enzyme sensing system comprises a covalently linked inhibitor-DNA-enzyme (IDE) complex, which can be similar to a previously described constructs, for example, as described by Saghatelian, et al. “DNA detection and signal amplification via an engineered allosteric enzyme”, J. Am. Chem. Soc. 125, 344-5 (2003); Gianneschi, et al. Design of molecular logic devices based on a programmable DNA-regulated semisynthetic enzyme, Angew. Chem. Int. Ed. Engl. 46, 3955-8 (2007), and the like).

As shown in FIG. 47, in this exemplary covalently linked inhibitor-DNA-enzyme (IDE) complex embodiment, in the initial inactive enzyme state, the catalytic site of the enzyme (cereus neutral protease (CNP)) is blocked by an inhibitor (phosphoramidite dipeptide) that is covalently tethered to a DNA aptamer molecule. In the presence of target molecules, the aptamer undergoes a conformational change by forming tertiary structure with the target molecule. This change of structure releases the inhibitor from the enzyme's catalytic site and allows for the sustained enzymatic reaction with a fluorogenic substrate. A single molecular recognition event can therefore be amplified thousands of times by continuous substrate turnover. By integrating a random sequence pool of DNA molecules into the IDE, the binding characteristics of a single DNA sequence can be coupled to the activity of the enzyme.

In alternative embodiments of the aptamer IDE system of the invention, the DNA can be a synthetic DNA or other nucleic acid, e.g., a synthetic, non-naturally occurring nucleotide or a nucleic acid analogue, such as a peptide nucleic acid (PNA) containing non-ionic backbones, oligonucleotides having phosphorothioate linkages, or oligonucleotides having synthetic DNA backbone analogues such as phosphoro-dithioate, methylphosphonate, phosphoramidate, alkyl phosphotriester, sulfamate, 3′-thioacetal, methylene(methylimino), 3′-N-carbamate, and morpholino carbamate nucleic acids.

In alternative embodiments of the aptamer IDE system of the invention, the complex can be designed to maximize “switching” (or on/off) ability; and libraries are designed to screen for aptamers of desired properties, for example, a structure where the γ-segment duplex dissociating is controlled by the aptamer binding affinity and the formed aptamer/target tertiary structure. Therefore, by incorporating γ-segment with different lengths in screening, aptamers with distinct affinity and switching efficiency are obtained. The dissociation of inhibitor from the catalytic site of the enzyme may or may not involve breaking the duplex DNA domain.

For example, in making exemplary IDE constructs for practicing this invention, the activity of each exemplary IDE construct can be measured in real-time by fluorescence detection in the presence of target ATP or thrombin (1 pM-100 μM). Addition of a 25-mer DNA complementary to the α-loop can be included as a positive control. Likewise, scrambled sequences in α-loop and GTP (for ATP) or albumin (for thrombin) can be used as negative controls. Outcome parameters used to quantify the performance of each aptamer IDE can include signal-to-background ratio, response time, sensitivity (or affinity, K_{d}), specificity and dynamic range. Kinetic parameters (K_{cat} and Kₘ) can be further determined by measuring the reaction kinetics between the IDE construct and the fluorogenic substrate at different concentration ranging from 1 nM to 500 μM to construct velocity-substrate curves.

In one embodiment, a poly(dimethylsiloxane) (PDMS) chip containing channels with depth of 15-50 μm and width of 30 μm is fabricated using standard soft lithography, and mounted on a glass microscope slide. The PDMS device can have one oil inlet and two aqueous inlets (one for IDE library solution with the other one for target and substrate). Standard pressure infuse/withdraw syringe pumps can be used to deliver reagents and oil at flow rates ranging from 0.5 to 2 μL/min. Uniform picoliter-sized droplets can be generated at a rate of approximately 1,000 Hz by flow focusing of the resulting stream with HFE-7500 fluorinated oil containing 2% (w/w) EA surfactant. Droplets can be generated with different sizes (5, 10, 20 and 30 μm in diameter), which can be easily achieved by tuning the microfluidic channel size and flow rate. For FACS sorting, the formed water-in-oil (W/O) single-emulsion droplets can be introduced into 2^{nd} microfluidic device with hydrophilic channels for the formation of water-in-oil-in-water (W/O/W) double-emulsion droplets. In order to minimize the effect of droplet generation time on the enzyme assay, a multi-layer microfluidic device that contains multiple, parallel droplet generating structures which is able to generate about 10⁷ droplets within several minutes can be used. Fluorescent droplets can be imaged and detected using a confocal microscope which consists of 488/561/633 nm argon lasers and PMT detectors. Droplets can be sorted by FACS using a BD FACSAria II™ cell sorter which typically operate at a throughput of >10⁷ droplets/hour.

For identifying a specific IDE for use in a particular assay or protocol, in one embodiment, an IDE library is encapsulated into droplets (which can be size optimized) using droplet microfluidics; for example, an initial library of about 10¹² molecules can be co-encapsulated with target molecules (ATP or glutamate) and fluorogenic enzyme substrate (DABCYL-βAla-Ala-Gly-Leu-Ala-βAla-EDANS in about 10⁷ drops (i.e., 10⁵ IDE/droplet). After incubation, the fluorescent droplets that contain aptamer(s) can be sorted by FACS. The correlation between droplet fluorescence and aptamer affinity and switching properties enables identification and sorting of aptamers with defined properties simply by adjusting FACS gating parameters. Sorted droplets can then be collected in an Eppendorf tube held on ice and subsequently broken by adding an equal volume of 1H,1H,2H,2H-Perfluoro-1-octanol (Aldrich). Fresh substrate-containing buffer can be added to dilute the solution and also to increase the separation efficiency from the oil phase. The aqueous phase can be collected and re-encapsulated. After this partitioning procedure, it can be expected that only a single molecule IDE is contained within any given droplet. Once the aptamer-containing droplets are identified by the fluorescent signal, they can be separated individually by FACS, e.g., to a 384 well plate. Finally, after the droplet is lysed in the well, single aptamer molecules can be PCR amplified from IDE directly, and can be sequenced. A negative selection component where the IDE library is first incubated with control molecules (a mixture of GTP, TTP and CTP for ATP; a mixture of glutamine and asparagine for glutamate) can be used to eliminate IDE molecules that are not completely inhibited in the initial stage or DNA sequences that can turn on fluorescence signal via cross-reactivity or nonspecific binding. This negative screening step can enable generation of aptamers that are highly specific to the targets.

The identified aptamer sequences can be characterized, e.g., the identified aptamer sequences can be validated individually to 1) ensure that they specifically bind to and are capable of switching in the presence of targets but not controls, and 2) identify the sequences that generate optimal properties (i.e., affinity, specificity, response time and switching efficiency). The fluorescence signals of each sensor can be monitored in the presence of target (e.g., ATP or glutamate) or their respective controls in a range of concentration (e.g., 1 pM to 100 μM) in real-time using a plate reader. This identifies key properties of identified aptamers/sensors including affinity (K_{d}), sensitivity, selectivity, signal/background ratio, response time, and dynamic range. Surface plasmon resonance (SPR) (BIAcore 3000™) can be used to further evaluate the binding kinetics (Kₒₙ and K_{off}) and reversibility of the identified aptamers. For example, this set of tests can identify a sensor construct for neurotransmitter imaging, e.g., identifying rapid ligand association and dissociation sensors that permit analysis of the transient (on the order of ms) pulses of neurotransmitters for synaptic transmission.

As illustrated in FIG. 48, in exemplary ENSNARA protocols, the rationale is that specific DNA sequences in the library (e.g., as aptamers, for this example) exist and, upon binding to the target molecule, undergo a conformational change to dissociate the inhibitor from the enzyme catalytic site therefore producing a fluorescence signal. In alternative embodiments, an initial library can contain greater than 10¹² IDE encapsulated in approximately 10⁷ drops (e.g., about 10⁵ IDE/droplet). The aptamer-containing droplets of this library will produce a fluorescent signal and are sorted. Subsequently, the drops will be broken, diluted, and re-encapsulated with target and fluorescent substrate in another 10⁷ droplets until only a single IDE molecule per droplet remains. Finally, the fluorescent droplets that contain aptamer IDE are sorted and the selected aptamers are sequenced.

In alternative embodiments, ENSNARA can utilize IDE with different structures, architectures and compositions. In alternative embodiments, ENSNARA can employ other signaling amplification processes including e.g., EXPonential Amplification Reaction (EXPAR). In alternative embodiments, ENSNARA can be optimized by numerous parameters including droplet size, reaction time and molecular concentrations in the droplet. In alternative, droplet size can range from between about 5 to 50 μm in diameter.

While the invention is not limited by any particular mechanism of action, in alternative ENSNARA embodiments:

(i) the aptamer conjugated to the IDE can dissociate the inhibitor from enzyme catalytic site to produce a fluorescent signal in response to the binding of target molecules. This is supported by:
(a) that the IDE system developed by Ghadiri and his coworkers is able to detect target complementary DNA using the same switching mechanism (Saghatelian, et al. DNA detection and signal amplification via an engineered allosteric enzyme. J. Am. Chem. Soc. 125, 344-5 (2003); Gianneschi, et al. Design of molecular logic devices based on a programmable DNA-regulated semisynthetic enzyme. Angew. Chem. Int. Ed. Engl. 46, 3955-8 (2007).), and
(b) that the structure-switching aptamers can change conformation from DNA duplex to aptamer/target complex upon target binding (see, e.g., Nutiu, R. & Li, Y, Structure-switching signaling aptamers. J. Am. Chem. Soc. 125, 4771-8 (2003); Tang, Z. et al. Aptamer switch probe based on intramolecular displacement. J. Am. Chem. Soc. 130, 11268-9 (2008)), and

ii) that the fluorescence signal triggered by a single aptamer switch can be detected in droplet due to the enzyme reporter signal amplification. This is supported by extensive previous studies including digital PCR and data presented in this invention that the compartmentalization of target enzymes in picoliter droplets permit single molecule detection by increasing the effective target concentration and signal-to-background ratio.

In alternative embodiments, exemplary ENSNARA systems and methods of the invention offer unparalleled sensitivity and throughput for rapid screening of aptamers with defined properties. In particular, the ability to detect single molecule in picoliter (pL)-sized droplet, and this invention's droplet “Break-Dilute-ReEncapsulate” partitioning procedure, allows direct screening of a library with a diversity of as high as approximately 10¹² in a single round. In alternative embodiments, exemplary ENSNARA circumvents the lengthy amplification steps necessitated by traditional SELEX (Systematic Evolution of Ligands by EXponential enrichment).

In alternative embodiments, once the aptamers are identified, they can be directly used as structure switching sensors without the need for additional modification and optimization66,68. In addition, the IDE system itself is not only a powerful aptamer screening platform but can also serve as a standalone, ultrasensitive and reversible sensor.

In alternative embodiments, the ENSNARA system or protocol of the invention is automated, e.g., in a microfluidic device; for example, by automating this system in a microfluidic device multiple targets can be selected for simultaneously.

In alternative embodiments, the ENSNARA systems or protocols of the invention comprise a single-round screening approach, which can circumvent a need for PCR amplification; and can also allow for the initial library to be composed of modified nucleotides, which can further increase the diversity and screening efficiency for high-quality aptamers.

In alternative embodiments, the ENSNARA systems or protocols of the invention comprise a new aptamer screening technology that can create a toolbox of real-time sensors for studying molecule and cellular signaling in vitro and in vivo, thus elucidating the biology and developing new therapeutics. In alternative embodiments, the ENSNARA systems or protocols of the invention comprise a rapid and reversible aptamer sensor system that permits continuous and real-time monitoring of neurotransmitters with high spatiotemporal resolution. In alternative embodiments, the ENSNARA systems or protocols of the invention comprise a platform for the design of many aptamers that can be used as probes to study complex biology, or as diagnostics and therapeutics.

A number of embodiments of the invention have been described. Nevertheless, it will be understood that various modifications may be made without departing from the spirit and scope of the invention. Accordingly, other embodiments are within the scope of the following claims.

1. A high throughput, multiplexed system or device, or method, for detecting, identifying and/or quantifying a target; a target molecule; a virus; a biological, a physiological or a pathological marker; a single molecule; or a single cell or cell-derived particle, optionally a single pathogen, parasite, bacterial cell, virus or fungus, using a droplet or emulsion microfluidics system, a 3D particle detector and/or a 3D particle counting system, or an emulsifier, integrated with use of an assay, a sensor or a sensing system comprising use of: a small molecule, a biomolecule, an aptamer, a DNAzyme, a nucleic acid, a protein, a peptide, an enzyme, an antibody, or a chemical or small molecule, comprising:
(a) providing an assay, a sensor, a detecting or a sensing system capable of specifically binding to or detecting directly or indirectly a target, a target molecule, a nucleic acid, a protein, a peptide, a virus, a cell-derived particle or a cell, wherein optionally the cell is a bacterial cell, a parasite cell or a fungal cell, or optionally the cell is a mammalian cell or a human cell;
wherein optionally the assay, sensor, detecting or sensing system comprises or comprises use of: an aptamer, a DNAzyme (also called a deoxyribozyme, a DNA enzyme or a catalytic DNA), a nucleic acid, a protein, a peptide, an enzyme, an antibody, or a chemical or small molecule, a single nucleic acid molecule amplification optionally comprising an EXPonential Amplification Reaction (EXPAR), a Rolling Circle Amplification (RCA), or an aptamer Inhibitor-DNA-Enzyme (IDE), or aptamer-IDE system,
and optionally the target comprises an amplified target, which optionally is a nucleic acid target amplified using Rolling Circle Amplification (RCA) or EXPAR,
wherein the specific binding to, or the direct or indirect detecting of, the target molecule, virus, cell-derived particle or cell, by the assay, sensor, detecting or sensing system results in, or generates, a detectable signal, which optionally comprises a fluorophore signal or a fluorescence,
wherein optionally the nucleic acid, aptamer, aptamer-IDE system, or DNAzyme comprises a RNA-cleaving DNA motif that can cleave a DNA-RNA chimeric substrate at a single ribonucleotide junction, and the ribonucleotide cleavage site is flanked by a fluorophore and a quencher, and optionally binding of the nucleic acid, aptamer, or DNAzyme to its target molecule, virus, cell-derived particle or cell causes cleavage of a ribonucleotide cleavage site to release the quencher from the fluorophore or a fluorescence activator, wherein the fluorescence activator optionally comprises an enzyme capable of when in active form generating a detectable signal such as a fluorophore signal,
and optionally the sensor or sensing system, aptamer, a DNAzyme, an aptamer inhibitor-DNA-enzyme (IDE) molecular complex (also called an aptamer-IDE system), which optionally comprises a structure as set forth in FIG. 47, wherein the enzyme of the IDE molecular complex when active or not under the influence of an inhibitor can generate a detectable signal such as a fluorescent signal when uninhibited, and the enzyme of the IDE molecular complex is inhibited by the inhibitor of the IDE molecular complex with the IDE molecular complex is not bound to a target, and the inhibitor of the IDE molecular complex is released, removed or deactivated from the enzyme when the aptamer of the IDE molecular complex binds its target, thus triggering activation of the enzyme and triggering the generation of the detectable signal or the fluorescent signal,
and optionally the assay, sensor, detecting or sensing system comprises a nucleic acid based, an antibody based, a protein based, a peptide based, an enzyme based or a chemical or small molecule-based assay, sensor, detecting or sensing system, or any combination thereof,
wherein optionally the specific binding of the assay, sensor, detecting or sensing system, to the target triggers an amplification-based or non-amplification-based fluorescence signal,
and optionally the target molecule, optionally a purified or complex target, can be screened, selected and/or isolated from a nucleic acid, peptide or chemical library,
and optionally the target molecule comprises a nucleic acid or a polypeptide, optionally the polypeptide is a diagnostic for a disease or condition, or is a cell surface marker, or is an enzyme,
wherein optionally the enzyme is a marker for the detection of a particular disease or is a marker, optionally the enzyme is a beta-lactamase, such as a carbapenemase, optionally for the detection of extended spectrum beta-lactamase (ESBL)-producing Enterobacteriaceae and carbapenem-resistant Enterobacteriaceae (CRE), TB and other antimicrobial resistant pathogens,
and optionally the target molecule, virus, cell-derived particle or cell or bacteria, parasite or fungus, comprises one or a plurality of biological, physiological or pathological markers, or comprises a single or a plurality of molecules or a single cell or a plurality of cells, or a single or a plurality of virus or a cell-derived particles or molecules;
(b) optionally providing a plurality of droplets, emulsions or microdroplets, wherein optionally the droplets, emulsions or microdroplets are generated by a droplet microfluidics system or a microdroplet-manipulating assay or device, an emulsifier, of an equivalent device or system,
and optionally droplet size can range from between about 5 to 50 μm in diameter, between about 1 μm to 300 μm, or between about 10 μm to 100 μm,
and optionally providing labels or stains, wherein optionally the target or the amplified target are stained or labeled, optionally with a dye, a nanoparticle, a bead, or an equivalent or combination thereof,
and optionally providing a plurality of particles or nanoparticles, wherein the target consists of, comprises or is contained in the particles or nanoparticles;
(c) providing a sample, wherein optionally the sample comprises or is derived from a biological or an environmental sample,
and optionally the sample comprises the target, or is suspected of comprising the target to be detected,
and optionally the target is or comprises a target molecule, a nucleic acid, a protein, a peptide, a virus, a cell-derived particle or a cell, wherein optionally the cell is a bacterial cell, a parasite cell or a fungal cell, or optionally the cell is a mammalian cell or a human cell;
(d) optionally encapsulating or microencapsulating the sample (comprising or consisting of the target), optionally together with the assay, sensor, detecting or sensing system,
and optionally associating, encasing, or binding the target or the sample with or within the plurality of particles or nanoparticles,
wherein optionally the encapsulating or microencapsulating comprises encapsulating or microencapsulating into a plurality of droplets or microdroplets, or emulsions,
and optionally the detecting or sensing system comprises an aptamer-IDE system, and optionally when the aptamer-IDE system comprises use of an enzyme, or a combination of enzymes, that can generate a detectable signal, such as a fluorescent signal, by interacting or processing the detectable signal, the encapsulating or microencapsulating further comprises encapsulating or microencapsulating a substrate or a detectable signal activated by the enzyme,
and optionally processing or making the encapsulated or microencapsulated sample or target, or processing or making the droplets or microdroplets, or emulsions, comprising the encapsulated or microencapsulated sample, comprises use of a droplet microfluidics system or microdroplet-manipulating device, or a high-throughput droplet generator, optionally a 256 channel cartridge system, or an emulsifier,
and optionally labeling or staining the target or the amplified target, optionally with a dye, a nanoparticle, a bead, or an equivalent or combination thereof; and
(e) detecting the presence of a detectable signal, which optionally comprises a fluorophore signal or a fluorescence, or a dye, a nanoparticle, a bead, or an equivalent or combination thereof,
wherein optionally the detecting, identifying and/or quantifying of the presence of a detectable signal is in each emulsified, encapsulated or microencapsulated sample, or in each droplet or microdroplet, or is in each particle or nanoparticle, and the detecting the presence of a detectable signal detects, identifies and/or quantifies the target molecule, virus, cell-derived particle or cell,
wherein optionally the cell is a mammalian cell, a human cell, a bacterial cell, a parasite cell, a fungal cell,
wherein the detection of a fluorophore signal or fluorescence, which optionally is in an encapsulated or microencapsulated sample, or a droplet or microdroplet, or an emulsion, or is in each particle or nanoparticle, indicates the presence of the target molecule, virus, cell-derived particle, cell, parasite, fungus or mammalian or human cell in the sample,
and optionally the detecting and/or quantifying the target molecule, a virus or a cell-derived particle or a cell comprises use of a 3D particle detector or a 3D particle counting system.

2. The high throughput, multiplexed system or device, or method, of claim 1, wherein the cell is a mammalian cell, a human cell, a cancer cell, a circulating tumor cell, a circulating prostate or melanoma cell, or a bacterial cell, optionally a slowly-growing organism such as Mycobacterium tuberculosis.

3. The high throughput, multiplexed system or method of claim 1, wherein the droplet or emulsion microfluidics system can generate:
(a) picoliter droplets or droplets of between about 1, 2, 3, 4, 5, 6, 7, 8, 9, or 10 μm to 300 μm, or between about 10 μm to 100 μm, in diameter; and/or
(b) monodisperse, picoliter-sized liquid droplets or emulsions in an immiscible carrier oil fluid.

4. The high throughput, multiplexed system or device, or method, of claim 1, wherein the biological sample comprises a biopsy, blood, serum, saliva, tear, urine or CSF sample from a patient, or a sample obtained from a food, water, soil, or an air source.

5. The high throughput, multiplexed system or device, or method, of claim 1, wherein the target molecule is or comprises a nucleic acid, a nucleic acid point mutation, or a single-nucleotide polymorphism (SNP), a microRNA (miRNA) or a small inhibitory RNA (siRNA), a cell marker or a marker specific or identifying for a particular cell type, genotype or phenotype; or a nucleic acid disease or cancer marker, optionally a breast cancer biomarker,
and optionally detection of the target molecule is diagnostic for the disease or cancer, or is used for routine disease or cancer screening, early stage disease or cancer diagnosis and/or prognosis, for monitoring disease or cancer progression and/or recurrence, and/or for monitoring drug effectiveness and safety.

6. The high throughput, multiplexed system or device, or method, of claim 1, wherein the target molecule is or comprises a protein, a lipid, a carbohydrate, a polysaccharide, a small molecule or a metal complex.

7. The high throughput, multiplexed system or device, or method, of claim 1, wherein the fluorophore comprises a fluorescein-dT and the quencher is a DABCYL-dT™ (Dabcyl-dT); and/or a fluorescence resonance energy transfer (FRET) dye pair; and/or a target-binding dye.

8. The high throughput, multiplexed system or device, or method, of claim 1, wherein the fluorescence is detected by an APD (photon avalanche diode), a PMT (photomultiplier tubes), a EMCCD (Electron Multiplying Charge Coupled Device), or a MCP (Microchannel plate) or other equivalent detector, optionally in a high throughput manner.

9. The high throughput, multiplexed system or device, or method, of claim 1, wherein the aptamer is an oligonucleotide, a nucleic acid or a peptide aptamer.

10. The high throughput, multiplexed system or device, or method, of claim 1, wherein the aptamer: specifically modulates stem cell differentiation into a particular lineage, or is directly coupled to a downstream signaling pathway, or the aptamer binds to a target as an agonist or as an antagonist or turns on a fluorescence signal as a sensor.

11. The high throughput, multiplexed system or device, or method, of claim 1, wherein the sensor comprises a DNA strand displacement strategy, a proximity ligation assay, or a binding induced DNA assembly assay, or equivalents; or, the sensor comprises a fluorogenic substrate or probe, or equivalents that binds to a target to produce fluorescence.

12. The high throughput, multiplexed system or device, or method, of claim 1, further comprising detecting and/or quantifying the biological, physiological or pathological maker, or single molecule or single cell integration comprising use of a 3D particle detector or a 3D particle counting system.

13. The high throughput, multiplexed system or device, or method, of claim 1, wherein the throughput, multiplexed system is engineered to comprise one or any of: desirable portability (for example, packaged as backpacks), automating fluid handing or droplet generation and auto sampling, and integrating electronics including a diode laser or a light source, APD (detector), Operating (vinci, ISS Inc.) and/or data analyzing software (SimFCS), display, with a 3D particle counting system, optionally as illustrated in FIGS. 32 and 33.

14. The high throughput, multiplexed system or device, or method, of claim 1, further comprising disposable microfluidic “cartridges,” permitting multiplex and rapid detection of multiple types of targets simultaneously, and optionally the high throughput, multiplexed system or device is fully automated, or is fabricated as an all-in-one system or with modular components, or is linked to an electronic device, a portable device, a smart phone and/or a Bluetooth, for point-of-care applications, optionally as illustrated in FIGS. 32, 33 and 40.

15. The high throughput, multiplexed system or device, or method, of claim 1, wherein the assay, sensor or sensor system comprises:
a nucleic acid based assay; an antibody based assay; an enzyme based assay; a chemical based assay; a nucleic acid based assay; a hybridization; a molecular beacon; an aptamer; a DNAzyme; a real-time fluorescent sensor; an antibody-based assay; an ELISA; a sandwich based assay; an immunostaining assay; an antibody capture assay; a secondary antibody amplification assay; a proximity ligation based assay; an enzyme based assay comprising use of a PCR, RT-PCR, RCA, loop-mediated isothermal amplification (LAMP), nicking, strand displacement and/or an exponential isothermal amplification; or any combination thereof,
wherein optionally the high throughput, multiplexed system or device, or method detects low concentration targets without using droplets,
and optionally nucleic acid targets are detected using signal amplification processes, optionally Rolling Circle Amplification (RCA),which are then stained by dye probes or nanoparticles and measured, optionally by a 3D particle counter.

16. The high throughput, multiplexed system or device, or method, of claim 1,
wherein the encapsulated or microencapsulated emulsions or droplets are made by using an emulsifier or by droplet based microfluidics; or
the emulsions or droplets comprise water-in-oil formulations, or the droplets comprise water-in-oil-in-water (W/O/W) double emulsion formulations, or the emulsions or droplets comprise liquid droplets,
optionally comprising an agarose or a PEG, or optionally the droplets can be gelled or solidified to form droplet particles;
and optionally droplets comprise sizes ranging from between about 10 nm to 100 microns, optionally droplets are monodispersed or polydispersed, and optionally droplets are heated or cooled, merged, split, sorted and/or prepared for long-term storage,
and optionally the emulsions or droplets, optionally fluorescent emulsions or droplets, that contain a target are sorted in a 3D particle counting system, optionally using an optical tweezer, an optical trap, an optical lattice, gradient centrifugation or any combination or equivalent thereof,
and optionally droplets are analyzed by conventional 1D on-chip or 2D analysis, or by a 3D particle counter.

17. The high throughput, multiplexed system or device, or method, of claim 1, wherein the cell-derived particle comprises an exosome, a microvesicle, an apoptotic body, or any combination thereof; or the target molecule comprises a nucleic acid, a protein, a peptide, a carbohydrate, a lipid, a small molecules, or a metal ion.

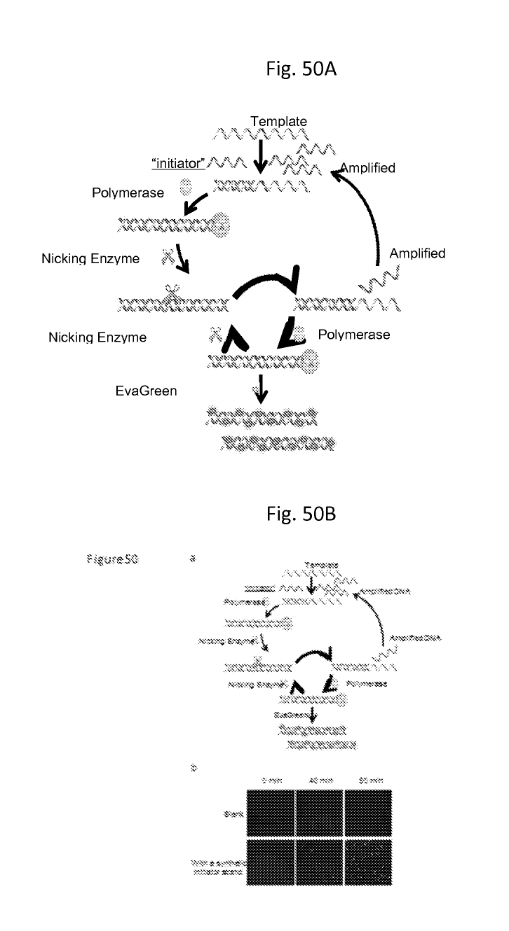

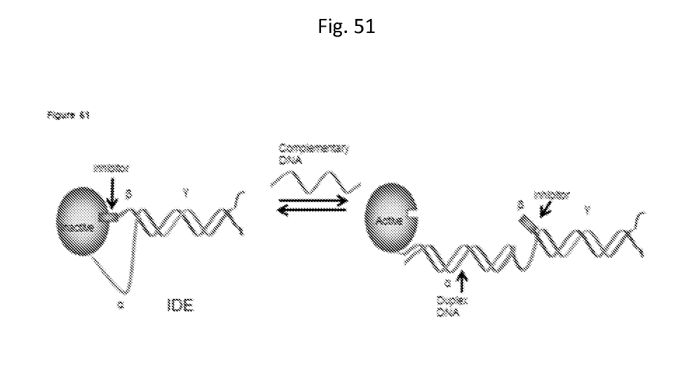

18. A method of identifying and isolating an enzyme-based target detection system for high through-put detection of specific target, comprising:
(a) providing a library of enzyme-based target detection system molecules designed to bind to and detect one specific target or a plurality of specific targets, the target to which the enzyme-based target detection system designed to detect, and a substrate comprising a detectable moiety,
wherein when the enzyme-based target detection system is not bound to its target, the enzyme is inactive,
and when the enzyme-based target detection system binds to its specific target, the enzyme is activated to act on the substrate to generate a detectable signal,
wherein optionally the generated detectable signal comprises a fluorescent signal,
and optionally the enzyme-based target detection system is an aptamer inhibitor-DNA-enzyme (IDE) system molecule, optionally as illustrated in FIG. 47 or FIG. 51A,
and optionally the enzyme-based target detection system is a nucleic acid initiator triggered signal amplification cascade, optionally as illustrated in FIG. 50;
(b) encapsulating the sample, an enzyme-based target detection system and substrate in an immiscible carrier oil fluid such that the encapsulation generates a plurality of droplets, wherein droplet each comprises a plurality of sample, an enzyme-based target detection system and substrate,
wherein optionally the encapsulating comprises pumping the sample, an enzyme-based target detection system and substrate through an oil stream, and optionally the plurality of droplets are picoliter sized droplets;
(c) passing the plurality of droplets generated in (b) through a sorter, which directs the droplets having a detectable signal into a separate channel where the sorted droplets are lysed or broken, diluted, and re-encapsulated with additionally added target and substrate at a concentration of about 1 enzyme-based target detection system molecule per drop with in each droplet one or more of substrate and target,
wherein optionally the sorted droplets are lysed or broken optionally using an optical tweezer, an optical trap, an optical lattice, gradient centrifugation or any combination or an equivalent thereof,
wherein optionally the generated detectable signal comprises a fluorescent signal and the sorter is a FACS,
and optionally the generated detectable signal comprises a fluorescent signal and the sorter is a microfluidic device; and
(d) further sorting out droplets having a detectable signal into a separate channel, thereby identifying and isolating an enzyme-based target detection system or molecule for high through-put detection of the specific target,
wherein optionally the enzyme-based target detection system or molecule comprises a aptamer inhibitor-DNA-enzyme (IDE) system molecule and the isolated IDE molecule is sequenced.

19. A drug or aptamer screening and in vitro selection platform based on one type of molecule/one bead or one type of molecule/one droplet strategy, wherein DNA, RNA, polypeptides and/or peptides are synthesized in a droplet library, comprising:
providing a high throughput, multiplexed system or device, of claim 1, and DNA on microbeads for generating a target or a binder to a target,
wherein the DNA on microbeads, or DNA-bread library, is used for screening drug or aptamer that possesses a function, e.g., binding to target molecule or modulate a molecular or cellular function, and optionally wherein the DNA on microbeads is encapsulated in the droplets or microdroplets, optionally picoliter droplets, optionally about 20 μm in diameter,
amplifying the on-bead DNA by PCR to generate a droplet DNA library,
transcribing and/or translating within the droplets the amplified DNA to form RNA and/or polypeptide or peptide libraries,
optionally the identity/sequence of transcribed RNA, and/or the translated polypeptides or peptides, are barcoded in the same droplet using the nucleic acid sequences, for subsequent screening and biomarker discovery,
and optionally the RNA and/or polypeptides or peptides are detected and/or quantified as the target using the high throughput, multiplexed system or device.

20. An Integrated Comprehensive Droplet Digital Detection (IC 3D) System comprising a system as set forth in FIGS. 17, 32 and 33.

21-22. (canceled)


SEQUENCE LISTING (11 sequences)

SEQ ID NO: 1 - DNA, 74 bp, artificial sequence
  synthetic polynucleotide
gtcagccatgagtaagcnnnnnnnnnnnnnnnnnnnnnnnnnnnnnnnnnnnnnnnngatctgtcgtctcactc

SEQ ID NO: 2 - protein, 29 aa, artificial sequence
  synthetic peptide
CTACTCGACAGATCQAFGCTTACTCTACG

SEQ ID NO: 3 - DNA, 24 bp, artificial sequence
  synthetic polynucleotide
ctcatggctgaccgtagagtaagc

SEQ ID NO: 4 - DNA, 17 bp, artificial sequence
  synthetic oligonucleotide
gtcagccatgagtaagc

SEQ ID NO: 5 - DNA, 17 bp, artificial sequence
  synthetic oligonucleotide
gagtgagacgacagatc

SEQ ID NO: 6 - DNA, 37 bp, artificial sequence
  synthetic oligonucleotide
aaaaaaaaaaaaaaaaaaaagagtgagacgacagatc

SEQ ID NO: 7 - DNA, 74 bp, artificial sequence
  synthetic oligonucleotide
gtcagccatgagtaagcgggaagcgtatagcctaaatgggatggacgtaccaacgaggatctgtcgtctcactc

SEQ ID NO: 8 - DNA, 74 bp, artificial sequence
  synthetic oligonucleotide
gtcagccatgagtaagcatcagcagcccactagataagtggagggaaagtctgtacagatctgtcgtctcactc

SEQ ID NO: 9 - DNA, 74 bp, artificial sequence
  synthetic oligonucleotide
gtcagccatgagtaagcggggagcgagtcatgagaaaatcgcggggaagcacagggtgatctgtcgtctcactc

SEQ ID NO: 10 - DNA, 74 bp, artificial sequence
  synthetic oligonucleotide
gtcagccatgagtaagcaattgatcgtggaaccagacgaataaaccacaggatttaggatctgtcgtctcactc

SEQ ID NO: 11 - DNA, 67 bp, artificial sequence
  synthetic oligonucleotide
cacggatcctgacaagnnnnnnnnnnnnnnnnnnnnnnnnnnnnnncagctccgtccgtcgattttt